SEQUENCE LISTING (15 sequences)

SEQ ID NO: 1 - DNA, 20 bp, Artificial Sequence
  Synthetic
ccagagcatccaaaagagtg

SEQ ID NO: 2 - DNA, 20 bp, Artificial Sequence
  Synthetic
tgaagtaaaaggagacaatt

SEQ ID NO: 3 - RNA, 80 nt, Artificial Sequence
  Synthetic
guuuuagagcuagaaauagcaaguuaaaauaaggcuaguccguuaucaacuugaaaaaguggcaccgagucggugcuuuu

SEQ ID NO: 4 - DNA, 102 bp, Artificial Sequence
  Synthetic
agagtctctcagctggtacagttttagagctagaaatagcaagttaaaataaggctagtccgttatcaacttgaaaaagtggcaccgagtcggtgctttttt

SEQ ID NO: 5 - DNA, 102 bp, Artificial Sequence
  Synthetic
ccagagcatccaaaagagtggttttagagctagaaatagcaagttaaaataaggctagtccgttatcaacttgaaaaagtggcaccgagtcggtgctttttt

SEQ ID NO: 6 - protein, 21 aa, Artificial Sequence
  Synthetic
MALPVTALLLPLALLLHAARP

SEQ ID NO: 7 - protein, 247 aa, Artificial Sequence
  Synthetic
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSS

SEQ ID NO: 8 - protein, 69 aa, Artificial Sequence
  Synthetic
TTTPAPRPPTPAPTIASQPLSLRPEACRPAAGGAVHTRGLDFACDIYIWAPLAGTCGVLLLSLVITLYC

SEQ ID NO: 9 - protein, 42 aa, Artificial Sequence
  Synthetic
KRGRKKLLYIFKQPFMRPVQTTQEEDGCSCRFPEEEEGGCEL

SEQ ID NO: 10 - protein, 112 aa, Artificial Sequence
  Synthetic
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - protein, 24 aa, Artificial Sequence
  Synthetic
SGGGGEGRGSLLTCGDVEENPGPR

SEQ ID NO: 12 - protein, 236 aa, Artificial Sequence
  Synthetic
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 13 - protein, 9 aa, Artificial Sequence
  Synthetic
LAGLIDADG

SEQ ID NO: 14 - protein, 6 aa, Artificial Sequence
  Synthetic
GIYYIG

SEQ ID NO: 15 - protein, 21 aa, Artificial Sequence
  Synthetic
MALPVTALLLPLALLLHAARP

(19) United States
(12) Patent Application Publication
(10) Pub. No.: US 2023/0036569 A1
(43) Pub. Date: Feb. 2, 2023

(54) ENGINEERED IMMUNE CELLS WITH REDUCED TOXICITY AND USES THEREOF

(71) Applicant: The General Hospital Corporation, Boston, MA (US)

(72) Inventor: Marcela V. Maus, Lexington, MA (US)

(73) Assignee: The General Hospital Corporation, Boston, MA (US)

(21) Appl. No.: 17/784,935

(22) PCT Filed: Dec. 17, 2020
(86) PCT No.: PCT/US2020/065733

§ 371 Date: Jun. 13, 2022

Related U.S. Application Data
(60) Provisional application No. 63/005,952, filed on Apr. 6, 2020.
(60) Provisional application No. 62/948,864, filed on Dec. 17, 2019.

(51) Int. Cl. A61K 35/17; C07K 14/725; C12N 15/11; C12N 9/22; C12N 15/113; C07K 14/705; A61P 35/00; A61P 35/02

(52) U.S. Cl. CPC A61K 35/17; C07K 14/7051; C12N 15/11; C12N 9/22; C12N 15/1136; C07K 14/70521; C07K 14/70578; C07K 14/70517; A61P 35/00; A61P 35/02; C07K 2319/02; C07K 2319/03; C07K 2317/622; C12N 2310/14; C12N 2310/20

(57) ABSTRACT

In the disclosure provided herein are engineered immune cells (e.g., T cells) deficient in INFy expression and have reduced toxicity. Methods of producing the engineered immune cells (e.g., T cells) and methods of using the engineered immune cells (e.g., T cells) to treat cancer or autoimmune disease are also provided.

### RELATED APPLICATIONS

This application claims the benefit under 35 U.S.C. § 119(e) to U.S. Provisional Application No. 62/948,864, filed Dec. 17, 2019, entitled “Neutralizing IFN gamma Production by CAR T Cells to Alleviate Cytokine Release Syndrome,” and U.S. Provisional Application No. 63/005,952, filed Apr. 6, 2020, entitled “ENGINEERED IMMUNE CELLS WITH REDUCED TOXICITY AND USES THEREOF,” the entire contents of each of which are incorporated herein by reference.

### BACKGROUND

Engineered T cells expressing chimeric antigen receptors (CAR) or engineered T cell receptors (TCRs) are effective treatment options for cancer and certain immune disorders. Side effects of such cell therapy include an acute systemic inflammatory syndrome (cytokine release syndrome).

### SUMMARY

The present disclosure is based, at least in part, on the surprising finding that IFNγ is not required for the therapeutic effects of CAR T cells, and further that CAR T cells deficient in IFNγ, while still are therapeutically effectively, stimulate less cytokine production and alleviates cytokine release syndrome associated with the therapy. Accordingly, provided herein are engineered immune cells with reduced toxicity, methods of engineering the immune cells, and methods of using the engineered immune cells, e.g., for the treatment of cancer or autoimmune diseases.

Some aspects of the present disclosure provide engineered immune cells comprising a chimeric antigen receptor (CAR) or an engineered T cell receptor (TCR), wherein the engineered immune cell is deficient in interferon γ (IFNγ) expression.

In some embodiments, the engineered immune cell comprises a nucleic acid comprising a nucleotide sequence encoding the CAR or the engineered TCR operably linked to a promoter.

In some embodiments, the immune cell comprises a nucleotide sequence that suppresses the IFNγ gene. In some embodiments, the nucleotide sequence that suppresses the IFNγ gene is a guide RNA (gRNA). In some embodiments, the gRNA comprises the nucleotide sequence of CCAGAGCATCCAAAAGAGTG (SEQ ID NO: 1) or TGAAGTAAAAGGAGACAATT (SEQ ID NO: 2). In some embodiments, the engineered immune cell further comprises a nucleotide sequence encoding a Cas9 nuclease or further comprises a Cas9 nuclease.

In some embodiments, the nucleotide sequence that suppresses the IFNγ gene encodes a RNAi molecule. In some embodiments, the RNAi molecule is a siRNA, a micro-RNA, a shRNA, or an antisense oligonucleotide. In some embodiments, the nucleotide sequence that suppresses the IFNγ gene is a ribozyme.

In some embodiments, the nucleotide sequence that suppresses the IFNγ gene encodes an enzyme selected from a transcription activator-like effector nuclease (TALEN), a zinc finger nuclease (ZFN), and a meganuclease.

In some embodiments, the nucleotide sequence that inactivates the IFNγ gene is encoded on the same nucleic acid comprising the nucleotide sequence encoding the CAR or the engineered TCR.

In some embodiments, the immune cell comprises an enzyme selected from a transcription activator-like effector nuclease (TALEN), a zinc finger nuclease (ZFN), and a meganuclease.

In some embodiments, the CAR comprises an extracellular antigen-binding domain, a transmembrane domain, and one or more intracellular signaling domains. In some embodiments, the extracellular antigen-binding domain comprises a single-chain antibody fragment (scFv) that binds a cell surface protein. In some embodiments, the extracellular antigen-binding domain binds CD19, BCMA, TACI, CD79b, CD22, CD30, CS1, GPCR, PSMA, mesothelin, MUC1, MUC16, EGFR, IL-13Ralpha2, EGFRvIII, CD20, CD79a, or combinations thereof. In some embodiments, the one or more intracellular signaling domains comprise (i) an ITAM-containing signaling domains and/or (ii) one or more signaling domains from one or more co-stimulatory proteins or cytokine receptors. In some embodiments, the ITAM-containing signaling domain is a CD3ζ signaling domain. In some embodiments, the co-stimulatory protein or cytokine receptor is CD28, 4-1BB, 2B4, KIR, CD27, OX40, ICOS, MYD88, IL2 receptor, or SynNotch. In some embodiments, the one or more intracellular signaling domains comprise (i) a signaling domain of CD3 and/or (ii) a signaling domain from CD28 or 4-1BB. In some embodiments, the transmembrane domain is a CD28 transmembrane domain or CD8 transmembrane domain. In some embodiments, the antigen binding domain further comprises a leader sequence.

In some embodiments, the immune cell is further deficient in endogenous TCR expression.

In some embodiments, wherein the immune cell is a T-cell, a NK cell, a dendritic cell, a macrophage, a B cell, a neutrophil, an eosinophil, a basophil, a mast cell, a myeloid-derived suppressor cell, a mesenchymal stem cell, a precursor thereof, or a combination thereof. In some embodiments, the immune cell is a T cell.

Further provided herein are nucleic acid molecules comprising: (i) a first nucleotide sequence encoding a chimeric antigen receptor (CAR) or an engineered T cell receptor (TCR); and (ii) a second nucleotide sequence encoding an agent that suppresses interferon γ (IFNγ) gene.

In some embodiments, the agent that suppresses IFNγ gene is a gRNA, a siRNA, a micro-RNA, a shRNA, an antisense oligonucleotide, a ribozyme, a transcription activator-like effector nuclease (TALEN), a zinc finger nuclease (ZFN), or a meganuclease. In some embodiments, the agent that suppresses IFNγ gene is a gRNA. In some embodiments, the gRNA comprises the nucleotide sequence of CCAGAGCATCCAAAAGAGTG (SEQ ID NO: 1) or TGAAGTAAAAGGAGACAATT (SEQ ID NO: 2).

In some embodiments, comprising a third nucleotide sequence encoding an agent that suppresses an endogenous TCR gene. In some embodiments, the agent that suppresses the endogenous TCR gene is a gRNA, a siRNA, a micro-RNA, a shRNA, an antisense oligonucleotide, a ribozyme, a transcription activator-like effector nuclease (TALEN), a zinc finger nuclease (ZFN), or a meganuclease. In some embodiments, the agent that suppresses endogenous TCR gene is a gRNA.

In some embodiments, the CAR comprises an extracellular antigen-binding domain, a transmembrane domain, and one or more intracellular signaling domains. In some embodiments, the extracellular antigen-binding domain comprises a single-chain antibody fragment (scFv) that binds a cell surface protein. In some embodiments, the extracellular antigen-binding domain binds CD19, BCMA, TACI, CD79b, CD22, CD30, CS1, GPCR, PSMA, mesothelin, MUC1, MUC16, EGFR, IL-13Ralpha2, EGFRvIII, CD20, CD79a, or combinations thereof. In some embodiments, the one or more intracellular signaling domains comprise (i) an ITAM-containing signaling domains and/or (ii) one or more signaling domains from one or more co-stimulatory proteins or cytokine receptors. In some embodiments, the ITAM-containing signaling domain is a CD3ζ signaling domain. In some embodiments, the co-stimulatory protein or cytokine receptor is CD28, 4-1BB, 2B4, KIR, CD27, OX40, ICOS, MYD88, IL2 receptor, or SynNotch. In some embodiments, the one or more intracellular signaling domains comprise (i) a signaling domain of CD3 and/or (ii) a signaling domain from CD28 or 4-1BB. In some embodiments, the transmembrane domain is a CD28 transmembrane domain. In some embodiments, the antigen binding domain further comprises a leader sequence.

In some embodiments, the nucleic acid is a vector. In some embodiments, the vector is an AAV, a lentiviral vector, or a retroviral vector.

Further provided herein are methods comprising delivering any one of the nucleic acids described herein to an immune cell. In some embodiments, the immune cell is a T-cell, a NK cell, a dendritic cell, a macrophage, a B cell, a neutrophil, an eosinophil, a basophil, a mast cell, a myeloid-derived suppressor cell, a mesenchymal stem cell, a precursor thereof, and a combination thereof. In some embodiments, the immune cell is a T cell.

In some embodiments, wherein the second nucleotide sequence encodes a gRNA and the method further comprises delivering to the immune cell a nucleotide sequence encoding a Cas9 nuclease or delivering to the immune cell a Cas9 nuclease.

Other aspects of the present disclosure provide methods of administering to a subject any one of the engineered immune cells described herein. In some embodiments, the method is for treating cancer or an autoimmune disease, and comprises administering to a subject in need thereof an effective amount of any one the engineered immune cell described herein. In some embodiments, the method is for reducing cytokine release associated with CAR-T cell therapy and comprises administering to a subject in need thereof an effective amount of any one the engineered immune cell described herein. In some embodiments, the subject is a human subject. In some embodiments, the administering is via infusion. In some embodiments, the engineered immune cell is allogeneic or autologous. In some embodiments, the level of inflammatory cytokines, chemokines, and/or adhesion molecules produced in the subject are reduced, compared to that of a subject administered an engineered immune cell not deficient in IFNγ expression. In some embodiments, the inflammatory cytokines, the chemokines, or the adhesion molecules are selected from: IL-4, IL-10, IL-12, IL-13, MIP1α, MIP1β, MCP1, IP10, E-selectin, P-selection, PSEL, IL-1beta, IL12p70 and SICAM1. In some embodiments, the reduction of the level of the inflammatory cytokines, chemokines, and/or adhesion molecules is in cancer microenvironment, circulation or central nervous system. In some embodiments, the cancer being treated is lymphoma. In some embodiments, the cancer being treated is mantle cell lymphoma. In some embodiments, the cancer being treated is leukemia. In some embodiments, the cancer being treated is acute lymphoblastic leukemia.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

### BRIEF DESCRIPTION OF THE DRAWINGS

The accompanying drawings are not intended to be drawn to scale. Copies of this patent or patent application publication with color drawing(s) will be provided by the Office upon request and payment of the necessary fee. In the drawings:

FIGS. 1A-1G show in vitro results following IFNγ blockade in CAR-T cells. (FIG. 1A) Schematic showing CAR T cell preparation method. (FIG. 1B) Percent inhibition of IFNγ production from CAR T cells activated with NALM6 cells (6 hours) by various doses of anti-IFNγ antibody relative to control cells. (FIG. 1C) Cytokine production by CAR T cells activated with NALM6 cells (6 hours) following IFNγ blockade, as measured by ELISA. (FIG. 1D) Cytokine production by CAR T cells 6 hours after activation with NALM6 cells following IFNγ blockade, as measured by Luminex. (FIG. 1E) CD69 expression and (FIG. 1F) CD107a expression on CAR T cells 6 hours after activation with NALM6 cells following IFNγ blockade, as measured by flow cytometry. (FIG. 1G) Quantification of NALM6 cell lysis in co-culture with CAR T cells following IFNγ blockade, at various ratios of effector CAR T cells to target cells and various concentrations of anti-IFNγ antibody used for blockade, as measured by luciferase assay 18 hours after cell mixing.

FIG. 2 shows lentiviral construct design. Each construct was designed to express a CD19 CAR fused to CD28 (top) or 4-1BB (bottom) and CD3ζ intracellular signaling domains, as well as mCherry.

FIGS. 3A-3D show depletion of IFNγ in CAR T cells. (FIG. 3A) Design of the lentiviral constructs used to express CD19 CARs and targeting the endogenous T cell receptor (TRAC) or IFNγ and TRAC. (FIG. 3B) Intracellular IFNγ expression in BBζ TRAC and BBζ TRAC IFNγ CAR T cells following a 6 hour activation with PMA/Ionomycin, measured by flow cytometry. (FIG. 3C) CD8 and intracellular IFNγ expression, as measured by flow cytometry, on CAR T cells activated with NALM6 or Jeko1 cells for 6 hours at the indicated E:T ratios. (FIG. 3D) Cytokine expression in BBζ TRAC and BBζ TRAC IFNγ CAR T cells stimulated with PMA and Ionomycin for 6 hours reported as the percent of CAR T cells (mCherry+ cells) positive for the cytokine stain, according to flow cytometric analysis.

FIGS. 4A-4D show the effects of IFNγ depletion in CAR T cells on CAR T cell killing of hematologic cancer cells in vitro. (FIG. 4A) Cell-specific lysis, measured by a luciferase-based killing assay, of NALM6 leukemia (left) or Jeko1 lymphoma (right) cancer cells by CD19-41BBζ CAR T cells after treatment with varying concentrations of anti-IFNγ, at the indicated E:T cellular ratios. (FIG. 4B) Cell-specific lysis, measured by a luciferase-based killing assay, of BCMA expressing MM.1s (left) or RPMI-8226 myeloma (right) cells by BCMA-4-1BBζ CAR T cells after treatment with varying concentrations of anti-IFNγ, at the indicated E:T ratios. (FIG. 4C) Cell-specific lysis, measured by a luciferase-based killing assay, of NALM6 (top) or Jako1 (bottom) cells by untransduced (UTD), BBζ TRAC, or BBζ TRAC IFNγ CAR T cells at the indicated E:T ratios. (FIG. 4D) Percent cytolysis, measured by ACEA assay, facilitated by untransduced (UTD), CD19-41BBζ, BBζ TRAC, or BBζ TRAC IFNγ CAR T cells. CD19-41BBζ (top) were treated with anti-IFNγ antibody. Cells were combined with NALM6 cells at varying E:T ratios and tracked by ACEA for 96 hours.

FIGS. 5A-5F show effects of IFNγ depletion in CAR T cells on CAR T cell killing of hematological cancer cells in vivo. (FIG. 5A) Schematic of treatment schedule for in vivo CAR T cell treatment in combination with anti-IFNγ antibody. (FIG. 5B) Average bioluminescence flux from mice injected with Jeko-1 CGB-GFP cancer cells and subsequently treated with untransduced (UTD) or CD19-41BBζ (19-BBζ) CAR T cells and treated with anti-IFNγ, control IgG antibody, or no antibody. (FIG. 5C) Bioluminescent images of mice treated according to the groups described in (FIG. 5B). (FIG. 5D) Schematic of treatment schedule for in vivo CAR T cell evaluation. (FIG. 5E) Average bioluminescence flux from mice injected with Jeko-1 CGB-GFP cancer cells and subsequently treated with untransduced (UTD) or CD19-41BBζ (19-BBζ) CAR T cells. (FIG. 5F) Bioluminescent images of mice treated according to the groups described in (FIG. 5E).

FIGS. 6A-6F show the effects of IFNγ depletion in CAR T cells on their interactions with macrophages. (FIG. 6A) Schematic showing the method of preparing CAR T cells and macrophages from the same healthy donor and subsequent stimulation and analysis. (FIG. 6B) IFNγ (circles) and IL-6 (triangles) measured over time in supernatant from co-cultures of CAR T cells, cancer (“target”) cells, and macrophages in a ratio of 50 T cells to 10 target cells to 1 macrophage. Macrophages were a mixture of M0 (M-CSF stimulated), M1 (GM-CSF, IFNγ, and LPS stimulated), and M2 (M-CSF, IL-4, and IL-13 stimulated), and cancer cells were NALM6 (top) or Jeko1 (bottom) cells. CAR T cells were transduced with CD19-BBζ TRAC or CD19-BBζ IFNγ TRAC constructs. (FIG. 6C) IFNγ and IL-6 measured over time in supernatant from co-cultures of CAR T cells, cancer cells, and macrophages in a ratio of 30 T cells to 30 target cells to 1 macrophage. Macrophages were a mixture of M0 (M-CSF stimulated), M1 (GM-CSF, IFNγ, and LPS stimulated), and M2 (M-CSF, IL-4, and IL-13 stimulated), and cancer cells were NALM6 (top) or Jeko1 (bottom) cells. CAR T cells were transduced with CD19-BBζ TRAC or CD19-BBζ IFNγ TRAC constructs. (FIG. 6D) mCherry quantification indicating number of CAR T cells over time (0 to 96 hours) in co-cultures with ratios of 1 CAR T cell:25 NALM6 tumor cells:0 or 1 macrophage (1E:25T or 1E:25T:1M), as measured by Incucyte. CAR T cells were detected by mCherry expression. (FIG. 6E) Representative images taken from NALM6 co-cultures with CAR T cells without anti-IFNγ antibody (1E:25T) or with CAR T cells and macrophages incubated with anti-IFNγ antibody (1E:25T:1M) at 96 hours. (FIG. 6F) Expression of PD-L1, measured by flow cytometry, on GFP+ cancer cells co-cultured for 96 hours with CAR T cells transduced with BBζ TRAC or BBζ TRAC IFNγ constructs, with (solid) or without (cross-hatched) macrophages. Cancer cells were NALM6 (left) or Jeko1 (right), and were mixed with CAR T cells and with or without macrophages at ratios of 1 CAR T cell:25 tumor cell:0 macrophages (1E:25T) or 1 CAR T cell:25 tumor cells:1 macrophage (1E:25T:1M).

FIGS. 7A-7J show effects of genetic deletion of IFNγ in CAR T cells on cytokine/chemokine production and adhesion molecule expression in the presence of macrophages. (FIGS. 7A-7E) Cytokine, chemokine, or adhesion molecule quantification in supernatant from NALM6 co-cultures with BBζ TRAC, or BBζ TRAC IFNγ CAR T cells and macrophages at a ratio of 50 T cells to 10 tumor cells to 1 macrophage (50E:10T:1M). (FIGS. 7F-7J) Cytokine, chemokine, or adhesion molecule quantification in supernatant from Jeko1 co-cultures with BBζ TRAC, or BBζ TRAC IFNγ CAR T cells and macrophages at a ratio of 50 T cells to 10 tumor cells to 1 macrophage. Supernatant was collected at 6, 24, 48, and 72 hours to measure cytokine and chemokine production and adhesion molecule expression using a Luminex kit.

FIGS. 8A-8C show the effect of IFNγ blockade in CAR T cells on the killing of glioblastoma cells. (FIG. 8A) Cell-specific cytolysis, measured by luciferase killing assay following overnight incubation of untransduced T cells or CAR T cells specific to the glioblastoma cell antigen EGFR with U87 (top) or U251 (bottom) glioblastoma cells at various effector to T cell (E:T) ratios. T cells were treated with specified doses of anti-IFNγ antibody prior to mixing with cancer cells. (FIG. 8B) Percent cytolysis of U87 (top) or U251 (bottom) cells over time, according to ACEA assay, measured following a 120-hour incubation of untransduced T cells (UTD) or EGFR CAR T cells with various concentrations of anti-IFNγ antibody. (FIG. 8C) Immunofluorescence images, taken on an Incucyte, of tumor cells and CAR T cells following a 96 hour incubation with anti-IFNγ antibody (replaced every 24 hours).

FIG. 9 shows effects of macrophages on expansion of CAR T cells when their expression of IFNγ is inhibited. Plots show relative CAR T cell proliferation, as measured on Incucyte by mCherry expression, when co-cultured with ASPC1 tumor cells at a ratio of 1 effector (CAR T cell) to 25 tumor cells (1E:25T) or ASPC1 tumor cells and macrophages at ratios of 1 effector (CAR T) cell to 25 tumor cells to 1 macrophage (1E:25T:1M), with or without different concentrations of anti-IFNγ antibody. CAR T cells were generated using the anti-mesothelin SS1 scFv (SS1-BBζ) or were untransduced (UTD) and were incubated with or without anti-IFNγ antibody for 96 hours.

FIGS. 10A-10B show the effect of IFNγ blockade in CAR T on the killing of pancreatic cancer cells. (FIG. 10A) Cell-specific lysis of ASPC1, BXPC3, or PANC1 pancreatic cancer cells, as measured by luciferase-based killing assay, following an overnight incubation with SS1-BBζ CAR T cells treated with or without different concentrations of anti-IFNγ antibody, at various ratios of CAR T (effector, E) cells to cancer (target, T) cells. (FIG. 10B) Percent cytolysis of ASPC1, BXPC3, or PANC1 pancreatic cancer cells, as measured by ACEA assay, following a 96-hour incubation with SS1-BBζ CAR T cells treated with or without different concentrations of anti-IFNγ antibody, at various ratios of CAR T cells to cancer cells (1:1, 1:5, or 1:25) over time (0 to 96 hours or 0 to 120 hours). For results shown in (B), anti-IFNγ antibody was added every 24 hours.

FIGS. 11A-11J show that IFNγ can be pharmaceutically blocked in CAR T cells. (FIG. 11A) T cells isolated from healthy donors were stimulated with beads coated in CD3 and CD28 antibodies for 24 hours before transducing with a lentiviral vector to express a CD19-41BBζ CAR (FIG. 11B). On Day 5, the stimulation beads were removed. On Day 9 or 10, the cells were treated with the indicated doses of anti-IFNγ antibody for one hour. (FIG. 11C) Cells were incubated with varying doses of anti-IFNγ or isotype control (0.25-20 ug/ml) for 1 hr at 37 C. After 1 hours, cells were activated with PMA/Ionomycin ×6 hours at 37 C. Cells were centrifuged, supernatant collected and ELISAs were performed using the Human DuoSet Kits from R&D (N=3). (FIG. 11D) CAR cells were expanded and transduced as shown in FIG. 11A, given varying doses of anti-IFNγ for 1 hr at 37 C and then treated with 10 ng/ml recombinant human IFNγ×20 minutes at 37 C. IFNγRa expression and pSTAT1 signaling were assessed by flow cytometry (N=3). (FIG. 11E) CAR-T were created using the protocol in FIG. 11A and transduction efficiency (mCherry) was checked by flow cytometry. (FIG. 11F) Cancer cell lines were given varying doses of anti-IFNγ for 1 hr at 37 C and then treated with 10 ng/ml recombinant human IFNγ×20 minutes at 37 C. IFNγRa expression and pSTAT1 signaling were assessed by flow cytometry (N=3). (FIG. 11G-H) Baseline IFNγRa expression on CAR-T and cancer cell lines was observed by flow cytometry (representative; N=3). (FIG. 11I) CAR-T viability following incubation with anti-IFNγ blocking antibody was examined at 6 hours post PMA/Ionomycin, 18 hours post-Nalm6 and 24 hours on resting T cells (N=5) (FIG. 11J) Cancer cells lines were incubated in IFNγ blocking antibody for 24 hours prior to assessing cell viability by flow cytometry (N=3 experiments).

FIGS. 12A-12G show that IFNγ can be genetically targeted in CAR T cells. (FIG. 12A) T cells isolated from healthy donors were stimulated with CD3 and CD28 beads for 24 hours before transducing with the lentiviral vectors in (FIG. 12B). On Day 5, the beads were removed and the cells were electroporated with Cas9 mRNA to initiate CRISPR-mediated deletion of the genes targeted by the guides (TRAC and/or IFNγ). On day 10, cells with successful deletion of TRAC were isolated by column purification of flow-based sorting for CD3⁺ cells. (FIG. 12B) Vector design for knockout CAR-T constructs (KO) with guide RNA to TRAC or TRAC and IFNγ. (FIG. 12C) KO CAR-T were activated with PMA/Ionomycin for 6 hours and cytokines were assessed by ELISA (N=3). (FIG. 12D) CAR-T were created using the protocol discussed in FIG. 11F and transduction efficiency was determined by flow cytometry. (FIG. 12E) KO CAR-T were assessed for CD3 expression pre- and post-CD3 isolation by flow cytometry (representative of N=5). (FIG. 12F) Baseline levels of IFNγRa on KO CAR-T was assessed by flow cytometry. (FIG. 12G) KO CAR-T were activated for 6 hours (PMA/Ionomycin) or 18 hours (Nalm6) before checking viability by flow cytometry (N=4).

FIGS. 13A-13G show that pharmacologic or genetic depletion of IFNγ does not reduce CAR T cell killing of hematologic cancer cell lines in vitro. (FIG. 13A) CD19-BBζ CAR T cells were produced as described and expanded for 14 days. Following treatment with varying concentrations of anti-IFNγ antibody, CAR T cells were activated with CD19-expressing NALM6 leukemia at the indicated E:T ratios overnight. Supernatant was collected and assessed for cytokine production by ELISA (N=5). (FIG. 13B) KO CAR T cells were produced as described and expanded for 14 days. CAR T cells were activated with CD19-expressing NALM6 leukemia at the indicated E:T ratios overnight. Supernatant was collected and assessed for cytokine production by ELISA (N=5). (FIGS. 13C-13D) Cell-specific lysis by CD19-BBζ CAR-T described in FIG. 13A was measured by a luciferase-based killing assay (FIG. 13C) or ACEA (D) (N=5). (FIGS. 13E-13F) Cell-specific lysis by KO CAR-T described in B was measured by a luciferase-based killing assay (FIG. 13E) or ACEA (FIG. 13F) (N=5). (FIG. 13G) Long-term killing of KO CAR-T was assessed by Incucyte at various tumor burdens: low (10E:1T), moderate (1E:1T) and high (1E:10T) (N=5). CD19-BBζ CART cells were produced as described and expanded for 14 days. Following treatment with varying concentrations of anti-IFNγ antibody, CAR T cells were activated with CD19-expressing NALM6 leukemia at the indicated E:T ratios overnight. Cell-specific lysis was measured by a luciferase-based killing assay (N=5).

FIGS. 14A-14I show that pharmacologic or genetic depletion of IFNγ does not reduce CAR T cell killing of leukemia in vivo. NSG mice were injected with 1e6 Nalm6 CBG-GFP cells IV. Seven days later, mice were injected with 1e6 CAR T cells IV and tumor burden was measured by bioluminescence. Bioluminescence was measured 4, 7, 14, 21, 28 and 35 days later. On these days, mice were also bled to look for the presence of CAR T cells. (FIGS. 14A-14D) Mice were injected with CD19-41BBζ CAR T cells and either not treated or treated with anti-IFNγ or control IgG antibody on the days indicated (N=3-5 mice/group; 4 donors). (FIG. 14B) Average bioluminescence flux each group of mice over time. (FIG. 14C) Bioluminescent images of the mice at each time point. (FIG. 14D) Mice were bled 3 days post-CAR-T and serum was tested for IFNγ expression by ELISA (N=3-5 mice/group; 4 donors). (FIGS. 14E-14I) Mice were injected with BBζ TRAC or BBζ TRAC IFNγ CAR T cells one week after Nalm6 injection. (FIG. 14F) Average bioluminescence flux each group of mice over time. (FIG. 14G) Bioluminescent images of the mice at each time point. (FIG. 14H) Mice were bled 3 days post-CAR-T, serum was collected and IFNγ was assessed by ELISA. (FIG. 14I) Mice were bled 14 days post-CAR-T injection and CAR-T persistence was determined by flow cytometry (N=3-5 mice/group; 4 donors).

FIGS. 15A-15H shows that pharmacologic or genetic depletion of IFNγ does not reduce CAR T cell killing of lymphoma in vivo. NSG mice were injected with 1e6 Jeko-1 CBG-GFP cells IV. Seven days later, mice were injected with 1e6 CAR T cells IV and tumor burden was measured by bioluminescence. Bioluminescence was measured 4, 7, 14, 21, 28 and 35 days later. On these days, mice were also bled to look for the presence of CAR T cells. (FIGS. 15A-15D) Mice were injected with CD19-41BBζ CAR T cells and either not treated or treated with anti-IFNγ or control IgG antibody on the days indicated (N=3-5 mice/group; 4 donors). (FIG. 15B) Average bioluminescence flux each group of mice over time. (FIG. 15C) Bioluminescent images of the mice at each time point. (FIG. 15D) Mice were bled 3 days post-CAR-T and serum was tested for IFNγ expression by ELISA (N=3-5 mice/group; 4 donors). (FIGS. 15E-15H) Mice were injected with BBζ TRAC or BBζ TRAC IFNγ CAR T cells 7 days post-Jeko-1 injection. (FIG. 15F) Average bioluminescence flux each group of mice over time. (FIG. 15G) Bioluminescent images of the mice at each time point. (FIG. 15H) Mice were bled 3 days post-CAR-T, serum was collected and IFNγ was assessed by ELISA.

FIGS. 16A-16E show that blocking IFNγ production by BBζ CAR-T reduces co-inhibitory marker expression and slightly enhances cell proliferation in vitro. (FIG. 16A) KO BBζ CAR-T were generated as previously described. Ten days post-activation, cells were re-activated with irradiated Jeko-1 or Nalm6 cells at a 1:1 ratio on days 0, 4 and 7. Cells were counted prior to each re-stimulation and proliferation doubling was calculated over time. For cultures receiving macrophages (dashed lines; bottom), monocytes were culture in GMCSF for 7 days prior to combination (N=5). (FIG. 16B) Prior to each re-activation, 1e5 cells were collected from cultures and analyzed by flow cytometry (N=5). (FIGS. 16C-16D) KO BBζ CAR-T were combined with Nalm6 cells at various tumor burdens (low=10E:1T, moderate=1E:1T, high=1E:10T). The bottom row was given 2,000 macrophages for a ratio of 1E:0.02M (N=5). (FIG. 16C) Changes in tumor burden were tracked by Incucyte using the average green area/well. (FIG. 16D) CAR-T expansion/contraction was monitored by Incucyte using the average area and fold change was calculated compared to hour 0 (N=5). (FIG. 16E) After 5 days in the Incucyte, cells were collected and stained for flow cytometry. Heatmap shows percent positive for each protein (gated on mCherry+ cells) (N=5).

FIGS. 17A-17E show that blocking IFNγ production by 28ζ CAR-T reduces co-inhibitory marker expression and greatly enhances cell proliferation in vitro. (FIG. 17A) KO 28ζ CAR-T were generated as previously described. Ten days post-activation, cells were re-activated with irradiated Jeko-1 or Nalm6 cells at a 1:1 ratio on days 0, 4 and 7. Cells were counted prior to each re-stimulation and proliferation doubling was calculated over time. For cultures receiving macrophages (dashed lines; bottom), monocytes were culture in GMCSF for 7 days prior to combination (N=5). (FIG. 17B) Prior to each re-activation, 1e5 cells were collected from cultures and analyzed by flow cytometry (N=5). (FIGS. 17C-17D) KO 28ζ CAR-T were combined with Nalm6 cells at various tumor burdens (low=10E:1T, moderate=1E:1T, high=1E:10T). The bottom row was given 2,000 macrophages for a ratio of 1E:0.02M (N=5). (FIG. 17C) Changes in tumor burden were tracked by Incucyte using the average green area/well. (FIG. 17D) CAR-T expansion/contraction was monitored by Incucyte using the average area and fold change was calculated compared to hour 0 (N=5). (FIG. 17E) After 5 days in the Incucyte, cells were collected and stained for flow cytometry. Heatmap shows percent positive for each protein (gated on mCherry+ cells) (N=5).

FIGS. 18A-18E show that genetic deletion of IFNγ in CAR T cells reduces cytokine/chemokine production and adhesion molecule expression in the presence of macrophages. T cells and monocytes were isolated from healthy donors. T cells were activated and transduced to express the KO CAR constructs as previously discussed. Monocytes were given GMCSF for 7 days for macrophage differentiation. (FIG. 18A) CAR-T and macrophages were combined with target cancer cells (Nalm6) at various tumor burden ratios: low (10E:1T:0.02M), moderate (1E:1T:0.02M) and high (1E:10T:0.02M). (FIG. 18B) Supernatant was collected 24, 48 and 72 hours post-combination. Cytokines/chemokines in the supernatant was collected and assayed by Luminex (N=4). (FIG. 18C) Fold change of FIG. 18B was calculated using TRAC divided by IFNγ TRAC. (FIG. 18D) T cells and monocytes were isolated from healthy donors and expanded into CAR-T and macrophages as mentioned above. CAR-T and Nalm6 cells were combined at a 1:1 ratio for 24 hours. After 24 hours, supernatant was collected and added directly to the donor-matched macrophages. Supernatant was collected 24 hours later. (FIG. 18E) Cytokines/chemokines in supernatant was collected and assayed by Luminex (N=1).

FIGS. 19A-19C show that IFNγ KO CAR-T yield less activation, IFNγ signaling and co-inhibitory molecules on macropages. T cells and monocytes were isolated from healthy donors and expanded into BBζ KO CAR-T and macrophages as mentioned above. CAR-T and Nalm6 cells were combined at a 1:1 ratio for 24 hours. After 24 hours, supernatant was collected and added directly to the donor-matched macrophages that had been plated on glass slides for 7 days in GMCSF (30,000 cells/well). After 48 hours, macrophages were stained for macrophage activation (FIG. 19A), IFNγ signaling (FIG. 19B) and co-inhibitory markers (FIG. 19C) and imaged using a Zeiss Observer Microscope (63×; N=1).

FIGS. 20A-20E show that serum from IFNγ KO CAR-T-treated mice yield less macrophage activation in vitro. NSG mice were treated with Nalm6 and KO CAR T cells as previously described. (FIG. 20A) Schematic of experiment showing that the serum collected from mice 3 days post-CAR-T injection is either saved directly for Luminex or added to donor-matched macrophages in vitro that were differentiated as previously discussed. (FIG. 20B) Serum from mice and from macrophages were collected 24 hours later and assessed by Luminex. (FIG. 20C) Fold change of FIG. 20B was calculated using TRAC/IFNγ TRAC. (FIG. 20D-20E) After collecting supernatant at 24 hours, macrophages were stained for IFNγ signaling (FIG. 20D) and co-inhibitory markers (FIG. 20E) and imaged using a Zeiss Observer Microscope (40×; N=2).

### DETAILED DESCRIPTION OF CERTAIN EMBODIMENTS

Engineered T cells expressing chimeric antigen receptors (CAR) or engineered T cell receptors (TCR) are effective treatment options for cancer and certain immune disorders, but they can have toxic side effects such as cytokine release syndrome. Endogenous cytotoxic T cells use ligand induced apoptosis, the release of perforin and granzyme, and IFNγ production to kill target cells. Whether engineered T cells such as CAR T cells or TCR T cells use these same mechanisms to kill cells expressing their cognate antigen remains unclear.

The present disclosure is based, at least in part, on the surprising finding that CAR T cells deficient in IFNγ expression may maintain therapeutic efficacy while simultaneously have reduced toxicity and cause fewer and less severe side effects. Accordingly, provided herein are engineered immune cells with reduced toxicity, methods of engineering the immune cells, and methods of using the engineered immune cells, e.g., for the treatment of cancer or autoimmune diseases.

Some aspects of the present disclosure provide an engineered immune cell comprising a chimeric antigen receptor (CAR) or an engineered T cell receptor (TCR), wherein the engineered immune cell is deficient in interferon γ (IFNγ) expression.

An “immune cell” can be a T-cell, an NK cell, a dendritic cell, a macrophage, a B cell, a neutrophil, an eosinophil, a basophil, a mast cell, a myeloid-derived suppressor cell, a mesenchymal stem cell, or combinations thereof, or any precursor, derivative, or progenitor cells thereof. In some embodiments, the immune cell is a T cell. An “engineered immune cell” refers to an immune cell that has been modified from its natural state. In some embodiments, modifications incorporated into an engineered immune cell include expression or introduction of exogenous nucleic acids, peptides, and/or proteins; depletion, activation, suppression, or editing of endogenous nucleic acids, peptides, and/or proteins; and/or treatment with a chemical or biological agent. In some embodiments, an engineered immune cell expresses a non-native marker or receptor, or expresses a modified or edited version of a native marker or receptor. In some embodiments, the engineered immune cell is an engineered T cell.

The engineered immune cell (e.g., engineered T cell) of the present disclosure is deficient in IFNγ expression. “Interferon γ (IFNγ)” is a dimerized soluble cytokine that is the only member of the type II class of interferons. IFNγ is a cytokine that is critical for innate and adaptive immunity. IFNγ is an important activator of macrophages and inducer of Class II major histocompatibility complex (MHC) molecule expression. IFNγ is produced predominantly by natural killer (NK) and natural killer T (NKT) cells as part of the innate immune response, and by CD4 Th1 and CD8 cytotoxic T lymphocyte (CTL) effector T cells once antigen-specific immunity develops. IFNγ is also produced by non-cytotoxic innate lymphoid cells (ILC). IFNγ is the primary cytokine that defines Th1 macrophage cells and is known for its biological significance in the immune system's antiviral, immunoregulatory, and anti-tumor properties.

Being “deficient in IFNγ expression” means that the engineered immune cell (e.g., engineered T cell) has reduced (e.g., reduced by at least 10%) relative expression level, or no expression of IFNγ. For example, the engineered immune cell (e.g., engineered T cell) may have an IFNγ expression level that is reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%, relative to a reference level. In some embodiments, the engineered immune cell (e.g., engineered T cell) has an IFNγ expression level that is reduced by about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99% or more, relative to a reference level. In some embodiments, the reference level is the IFNγ expression level in an unmodified immune cell (e.g., an unmodified T cell).

In some embodiments, the engineered immune cell comprises a nucleotide sequence that suppresses (e.g., deletes, modifies, or down-regulates) the IFNγ gene. Any method known in the art for suppressing (down-regulating) the expression of an endogenous gene in a host cell can be used to reduce the production level IFNγ as described herein. Suppression of the IFNγ gene can be transient (e.g., through RNA interference) or permanent (e.g., via gene editing).

In some embodiments, a gene editing method can be performed to modify the IFNγ gene (e.g., in a coding region or a non-coding regulatory region) so as to reduce expression of the target endogenous cytokine(s). A gene editing method may involve the use of an endonuclease that is capable of cleaving the target region in the endogenous allele. Non-homologous end joining in the absence of a template nucleic acid may repair double-strand breaks in the genome and introduce mutations (e.g., insertions, deletions and/or frameshifts) into a target site. Gene editing methods are generally classified based on the type of endonuclease that is involved in generating double stranded breaks in the target nucleic acid. Examples include, but are not limited to, Clustered Regularly Interspaced Short Palindromic Repeats (CRISPR)/endonuclease systems, transcription activator-like effector-based nuclease (TALEN), zinc finger nucleases (ZFN), endonucleases (e.g., ARC homing endonucleases), meganucleases (e.g., mega-TALs), or a combination thereof.

Cleavage of a gene region may comprise cleaving one or two strands at the location of the target IFNγ allele by an endonuclease. In some embodiments, the cleavage event may be followed by repairing the cleaved target polynucleotide by homologous recombination with an exogenous template polynucleotide, leading to insertion, deletion, or substitution of one or more nucleotides of the target nucleotide sequence. Such gene editing can result in decreased transcription the IFNγ gene.

The reduction level of the IFNγ in an immune cell population can be modulated by the level of gene editing event introduced into the cell population. For example, a large amount of one or more gene editing components introduced into a population of immune cells would result in a large portion of the immune cells having the target IFNγ allele edited. As such, the total production level of IFNγ would be reduced by a high level. Alternatively, a small amount of one or more gene editing components introduced into a population of immune cells would result in a small portion of the immune cells having the target IFNγ allele edited. As such, the total production level of IFNγ would be reduced by a low level. Thus, controlling the amount of one or more gene editing components to be delivered to a population of immune cells could control the total reduction level of IFNγ. Other suitable approaches may also be applicable to control the reduction level of IFNγ, as known to those skilled in the art.

In some embodiments, genetic modification that suppresses the IFNγ gene of immune cells as described herein is performed using the Clustered Regularly Interspaced Short Palindromic Repeats (CRISPR)/endonuclease technology known in the art. The CRISPR/endonuclease systems have been adapted for use in both prokaryotic and eukaryotic cells. Gene editing with CRISPR generally relies on expression of at least two components: a guide RNA sequence that recognizes a target nucleic acid sequence and an endonuclease (e.g., including Cpf1 and Cas9). A guide RNA helps direct an endonuclease to a target site, which typically contains a nucleotide sequence that is complementary (partially or completely) to the gRNA or a portion thereof. In some embodiments, the guide RNA is a two-piece RNA complex that comprises a protospacer fragment that is complementary to the target nuclei acid sequence and a scaffold RNA fragment. In some embodiments, the scaffold RNA is required to aid in recruiting the endonuclease to the target site. In some embodiments, the guide RNA is a single guide RNA (sgRNA) that comprises both the protospacer sequence and the scaffold RNA sequence. An exemplary sequence of the scaffold RNA can be: GUUUUAGAGCUAGAAAUAGCAAGUUAAAAUAAGGCUAGUCCGUUAUCAACUUG AAAAAGUGGCACCGAGUCGGUGCUUUU (SEQ ID NO: 3). Once at the target site, the endonuclease can generate a double strand break. It would have been known to those skilled in the art that nucleotide sequences for RNA molecules include residue “U.” The corresponding DNA sequence of any of the RNA sequences disclosed herein is also within the scope of the present disclosure. Such a DNA sequence would include “T” in replacement of “U” in the corresponding RNA sequence.

The target nucleic acid for use with the CRISPR system is flanked on the 3′ side by a protospacer adjacent motif (PAM) that may interact with the endonuclease and be further involved in targeting the endonuclease activity to the target nucleic acid. It is generally thought that the PAM sequence flanking the target nucleic acid depends on the endonuclease and the source from which the endonuclease is derived. For example, in some embodiments, for Cas9 endonucleases that are derived from Streptococcus pyogenes, the PAM sequence is NGG. In some embodiments, for Cas9 endonucleases derived from Staphylococcus aureus, the PAM sequence is NNGRRT. In some embodiments, for Cas9 endonucleases that are derived from Neisseria meningitidis, the PAM sequence is NNNNGATT. In some embodiments, for Cas9 endonucleases derived from Streptococcus thermophilus, the PAM sequence is NNAGAA. In some embodiments, for Cas9 endonuclease derived from Treponema denticola, the PAM sequence is NAAAAC. In some embodiments, for a Cpf1 nuclease, the PAM sequence is TTN.

A CRISPR/endonuclease system that hybridizes with a target sequence in the locus of an endogenous cytokine may be used to knock out the cytokine of interest. In some embodiments, the nucleotide sequence that suppresses the IFNγ is a guide RNA (gRNA) that hybridizes to (complementary to, partially or completely) a target nucleic acid sequence (e.g., the endogenous locus of a cytokine) in the IFNγ gene in the immune cell. For example, the gRNA or portion thereof may hybridize to the IFNγ gene with a hybridization region of between 15-25 nucleotides, 18-22 nucleotides, or 19-21 nucleotides in length. In some embodiments, the gRNA sequence that hybridizes to the IFNγ gene is 15, 16, 17, 18, 19, 20, 21, 22, 23, 24, or 25 nucleotides in length. In some embodiments, the gRNA sequence that hybridizes to the IFNγ gene is between 10-30, or between 15-25, nucleotides in length.

In some embodiments, the gRNA sequence is at least 50%, 55%, 60%, 65%, 70%, 75%, 80%, 85%, 90%, 95%, 96%, 97%, 98%, 99%, or at least 100% complementary to a target nucleic acid such as a region in the IFNγ gene (see also U.S. Pat. No. 8,697,359, which is incorporated by reference for its teaching of complementarity of a gRNA sequence with a target polynucleotide sequence). It has been demonstrated that mismatches between a CRISPR guide sequence and the target nucleic acid near the 3′ end of the target nucleic acid may abolish nuclease cleavage activity (see, e.g., Upadhyay, et al. Genes Genome Genetics (2013) 3(12):2233-2238). In some embodiments, the gRNA sequence is at least 50%, 55%, 60%, 65%, 70%, 75%, 80%, 85%, 90%, 95%, 96%, 97%, 98%, 99%, or at least 100% complementary to the 3′ end of the target region in the IFNγ gene (e.g., the last 5, 6, 7, 8, 9, or 10 nucleotides of the 3′ end of the target nucleic acid).

The “percent identity” of two nucleic acids is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST nucleotide searches can be performed with the NBLAST program, score=100, wordlength-12 to obtain nucleotide sequences homologous to the nucleic acid molecules of the invention. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In some embodiments, the gRNA that targets the IFNγ gene comprises a nucleotide sequence of CCAGAGCATCCAAAAGAGTG (SEQ ID NO: 1) or TGAAGTAAAAGGAGACAATT (SEQ ID NO: 2).

A variety of CRISPR/endonuclease systems are known in the art and modifications are regularly made, and numerous references describe rules and parameters that are used to guide the design of CRISPR/endonuclease systems (e.g., including Cas9 target selection tools). See, e.g., Hsu et al., Cell, 157(6):1262-78, 2014.

When the CRISPR system is used for modification of the immune cells, the immune cells may comprise a gRNA (e.g., encoded on a nucleic acid vector such as a plasmid or a viral vector) and the Cas9 nuclease may be additionally provided to the immune cell. In some embodiments, the Cas9 nuclease is provided transiently, e.g., by delivering to the immune cell comprising the gRNA a messenger RNA (mRNA) encoding Cas9 for transient expression. In some embodiments, the Cas9 and the gRNA targeting the IFNγ gene are delivered (e.g., by electroporation) into the immune cells as a complex (e.g., a complex that is isolated in vitro).

In some embodiments, genetic modification that suppresses the IFNγ gene of the immune cells as described herein is performed using the TALEN technology known in the art. TALENs are engineered restriction enzymes that can specifically bind and cleave a desired target DNA molecule. A TALEN typically contains a Transcriptional Activator-Like Effector (TALE) DNA-binding domain fused to a DNA cleavage domain. The DNA binding domain may contain a highly conserved 33-34 amino acid sequence with a divergent 2 amino acid RVD (repeat variable dipeptide motif) at positions 12 and 13. The RVD motif determines binding specificity to a nucleic acid sequence and can be engineered according to methods known to those of skill in the art to specifically bind a desired DNA sequence (see, e.g., Juillerat et al., Scientific reports, 5:8150, 2015; Miller et. al., Nature Biotechnology 29 (2): 143-8, 2011; Zhang et. al. Nature Biotechnology 29 (2): 149-53, 2011; Geipler et al., PLoS ONE 6 (5): e19509, 2011; Boch, Nature Biotechnology 29 (2): 135-6, 2011; Boch, et. al., Science 326 (5959): 1509-12, 2009; and Moscou et al., Science 326 (5959): 1501, 2009. The DNA cleavage domain may be derived from the FokI endonuclease, which is active in many different cell types. The FokI domain functions as a dimer, requiring two constructs with unique DNA binding domains for sites in the target genome with proper orientation and spacing. Both the number of amino acid residues between the TALE DNA binding domain and the FokI cleavage domain and the number of bases between the two individual TALEN binding sites appear to be important parameters for achieving high levels of activity. See, e.g., Miller et al., Nature Biotech. 29: 143-8, 2011.

TALENs specific to sequences in the IFNγ gene can be constructed using any method known in the art, including various schemes using modular components. See, e.g., Zhang et al. Nature Biotech. 29, 2011: 149-53; and Geibler et al., PLoS ONE 6: e19509, 2011. A TALEN specific to a sequence in the IFNγ gene can be used inside an immune cell to produce a double-stranded break (DSB). A mutation can be introduced at the break site if the repair mechanisms improperly repair the break via non-homologous end joining. For example, improper repair may introduce a frame shift mutation.

When the TALEN is used for modification of the immune cells, the immune cells may comprise a nucleotide sequence encoding the TALEN that targets the IFNγ gene. In some embodiments, the TALEN is provided transiently, e.g., by delivering to the immune cell a mRNA encoding the TALEN for transient expression. In some embodiments, the TALEN is delivered (e.g., by electroporation) into the immune cells as isolated proteins (e.g., TALEN that is isolated in vitro).

In some embodiments, zinc finger nucleases (ZFNs), which are known in the art, may be used to generate the engineered immune cells described herein. Zinc finger nucleases (ZFNs) are restriction enzymes comprised of an engineered zinc finger DNA binding domain linked to the catalytic domain of the type II endonuclease FokI. The zinc finger DNA binding domain of each ZFN targets the linked FokI endonuclease to a specific site in the genome.

Since FokI functions only as a dimer, a pair of ZFNs is typically engineered to bind to cognate target “half-site” sequences on opposite DNA strands. The target “half-site” sequences are generally spaced such the catalytically active FokI dimer may form between them. Upon dimerization of the FokI domain, a DNA double-strand break is generated between the ZFN half-sites. As mentioned above, non-homologous end joining may introduce mutations, while homology-directed repair may be used to introduce an exogenous nucleic acid. Many gene editing systems using ZFNs and considerations for design of ZFNs have been described; see, e.g., Segal et al., Proc Natl Acad Sci USA 96(6):2758-63, 1999; Dreier B et al., J Mol Biol. 303(4):489-502, 2000; Liu Q et al., J Biol Chem. 277(6):3850-6, 2002; Dreier et al., J Biol Chem 280(42):35588-97, 2005; and Dreier et al., J Biol Chem. 276(31):29466-78, 2001.

When the ZFN is used for modification of the immune cells, the immune cells may comprise a nucleotide sequence encoding the ZFN that targets the IFNγ gene. In some embodiments, the ZFN is provided transiently, e.g., by delivering to the immune cell a mRNA encoding the ZFN for transient expression. In some embodiments, the ZFN is delivered (e.g., by electroporation) into the immune cells as isolated proteins (e.g., ZFN that is isolated in vitro).

Meganucleases (or homing endonucleases), which are sequence-specific endonucleases that recognize long DNA targets (often between 14 and 40 base pairs) may also be introduced using any method known in the art to genetically engineer any of the immune cells described herein. There are at least six families of meganucleases and they are often classified based on structural motifs, including LAGLIDADG (SEQ ID NO: 13), GIY-YIG (SEQ ID NO: 14), HNH, His-Cys box, PD-(D/E)XK and Vsr-like. Non limiting examples of meganucleases include PI-SceI, I-CreI and I-TevI.

Various gene editing systems using meganucleases, including modified meganucleases, have been described in the art; see, e.g., the reviews by Steentoft et al., Glycobiology 24(8):663-80, 2014; Belfort and Bonocora, Methods Mol Biol. 1123:1-26, 2014; Hafez and Hausner, Genome 55(8):553-69, 2012; and references cited therein.

Hybrid nucleases including MegaTAL may also be used. MegaTALs are a fusion of a TALE DNA binding domain with a catalytically active meganuclease. Such nucleases harness the DNA binding specificity of TALEs and the sequence cleavage specificity of meganucleases. See, e.g., Boissel et al., NAR, 42: 2591-2601, 2014.

In some embodiments, the nucleotide sequence that suppresses the IFNγ gene in the engineered immune cells described herein is a RNAi molecule targeting the IFNγ mRNA, for example, without limitation, siRNA, dsRNA, stRNA, shRNA, microRNA (miRNA), antisense oligonucleotides, and modified versions thereof. In general, RNA interference technology is well known in the art, as are methods of delivering RNA interfering agents. See, e.g., U.S. Patent Pub. No. 2010/0221226.

As used herein, the term “RNAi” refers to any type of interfering RNA, including but are not limited to, siRNA, shRNA, endogenous microRNA and artificial microRNA. For instance, it includes sequences previously identified as siRNA, regardless of the mechanism of down-stream processing of the RNA (i.e., although siRNAs are believed to have a specific method of in vivo processing resulting in the cleavage of mRNA, such sequences can be incorporated into the vectors in the context of the flanking sequences described herein.

As used herein an “siRNA” refers to a nucleic acid that forms a double stranded RNA, which double stranded RNA has the ability to reduce or inhibit expression of a gene or target gene when the siRNA is present or expressed in the same cell as the target gene, for example where a target gene is IFNγ. The double stranded RNA siRNA can be formed by the complementary strands. In one embodiment, a siRNA refers to a nucleic acid that can form a double stranded siRNA. The sequence of the siRNA can correspond to the full length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length.

As used herein “shRNA” or “small hairpin RNA” (also called stem loop) is a type of siRNA. In one embodiment, these shRNAs are composed of a short, e.g., about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

A stem-loop structure refers to a nucleic acid having a secondary structure that includes a region of nucleotides which are known or predicted to form a double strand (stem portion) that is linked on one side by a region of predominantly single-stranded nucleotides (loop portion). The terms “hairpin” and “fold-back” structures are also used herein to refer to stem-loop structures. Such structures are well known in the art and the term is used consistently with its known meaning in the art. The actual primary sequence of nucleotides within the stem-loop structure is not critical to the practice of the disclosure as long as the secondary structure is present. As is known in the art, the secondary structure does not require exact base-pairing. Thus, the stem may include one or more base mismatches. Alternatively, the base-pairing may be exact, i.e. not include any mismatches. In some embodiments the precursor microRNA molecule may include more than one stem-loop structure. The multiple stem-loop structures may be linked to one another through a linker, such as, for example, a nucleic acid linker or by a microRNA flanking sequence or other molecule or some combination thereof. The actual primary sequence of nucleotides within the stem-loop structure is not critical as long as the secondary structure is present. As is known in the art, the secondary structure does not require exact base-pairing. Thus, the stem may include one or more base mismatches. Alternatively, the base pairing may not include any mismatches.

Any of the antisense oligonucleotides or an expression cassette for producing such may be delivered into immune cells via conventional methods to down-regulate the production of IFNγ as described herein. In some embodiments, the nucleotide sequence that suppresses the IFNγ gene in the engineered immune cells described herein is a ribozyme.

In some embodiments, any one of the engineered immune cells that is deficient in IFNγ expression is further modified to express an heterologous protein, for example, without limitation, an engineered receptor such as a chimeric antigen receptor (e.g., a chimeric synNotch receptor, a chimeric immunoreceptor, a chimeric costimulatory receptor, a chimeric killer-cell immunoglobulin-like receptor (KIR)) or an engineered T cell receptor (TCR), and/or having the endogenous TCR knocked out.

In some embodiments, any one of the engineered immune cells that is deficient in IFNγ expression described herein further comprises a CAR. A “chimeric antigen receptor” refers to a receptor protein that has been engineered to perform both antigen-binding and cell activating functions. In some embodiments, a CAR comprises a plurality of linked domains having distinct functions. CAR domains include those with antigen-binding functions, those with structural functions, and those with signaling functions. In some embodiments, a CAR comprises at least an extracellular ligand domain, a transmembrane domain and a cytoplasmic signaling domain (also referred to herein as “an intracellular signaling domain”) comprising a functional signaling domain derived from a stimulatory molecule as defined below. In some embodiments, the CAR comprises an optional leader sequence, an extracellular antigen binding domain, a hinge, a transmembrane domain, and an intracellular stimulatory domain. In some embodiments, the domains in the CAR polypeptide construct are in the same polypeptide chain, e.g., comprise a chimeric fusion protein. In some embodiments, the domains in the CAR polypeptide construct are not contiguous with each other.

In some embodiments, the CAR described herein comprises an extracellular domain. In some embodiments, the extracellular domain comprises an antigen binding domain. In some embodiments, antigen binding domain comprises an immunoglobulin chain or fragment thereof, comprising at least one immunoglobulin variable domain sequence. The term “antigen binding domain” encompasses antibodies and antibody fragments. In some embodiments, an antibody molecule is a multispecific antibody molecule, e.g., it comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In some embodiments, a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope.

The antigen binding domain can be any protein that binds to the antigen including but not limited to a monoclonal antibody, a polyclonal antibody, a recombinant antibody, a human antibody, a humanized antibody, and a functional fragment thereof, including but not limited to a single-chain variable fragment (scFV), a single-domain antibody such as a heavy chain variable domain (VH), a light chain variable domain (VL) and a variable domain (VHH) of camelid derived nanobody, and to an alternative scaffold known in the art to function as antigen binding domain, such as a recombinant fibronectin domain, and the like. In some embodiments, it is beneficial for the antigen binding domain to be derived from the same species in which the CAR will ultimately be used in. For example, for use in humans, it may be beneficial for the antigen binding domain of the CAR to comprise human or humanized residues for the antigen binding domain of an antibody or antibody fragment. In some embodiments, the antigen binding domain comprises a scFv that binds to a target antigen.

In some embodiments, the antigen binding domain comprises a human antibody or an antibody fragment. In some embodiments, the antigen binding domain comprises a humanized antibody or an antibody fragment. A humanized antibody can be produced using a variety of techniques known in the art, including but not limited to, CDR-grafting (see, e.g., European Patent No. EP 239,400; International Publication No. WO 91/09967; and U.S. Pat. Nos. 5,225,539, 5,530,101, and 5,585,089, each of which is incorporated herein in its entirety by reference), veneering or resurfacing (see, e.g., European Patent Nos. EP 592,106 and EP 519,596; Padlan, 1991, Molecular Immunology, 28(4/5):489-498; Studnicka et al., 1994, Protein Engineering, 7(6):805-814; and Roguska et al., 1994, PNAS, 91:969-973, each of which is incorporated herein by its entirety by reference), chain shuffling (see, e.g., U.S. Pat. No. 5,565,332, which is incorporated herein in its entirety by reference), and techniques disclosed in, e.g., U.S. Patent Application Publication No. US2005/0042664, U.S. Patent Application Publication No. US2005/0048617, U.S. Pat. Nos. 6,407,213, 5,766,886, International Publication No. WO 9317105, Tan et al., J. Immunol., 169:1119-25 (2002), Caldas et al., Protein Eng., 13(5):353-60 (2000), Morea et al., Methods, 20(3):267-79 (2000), Baca et al., J. Biol. Chem., 272(16):10678-84 (1997), Roguska et al., Protein Eng., 9(10):895-904 (1996), Couto et al., Cancer Res., 55 (23 Supp):5973s-5977s (1995), Couto et al., Cancer Res., 55(8):1717-22 (1995), Sandhu J S, Gene, 150(2):409-10 (1994), and Pedersen et al., J. Mol. Biol., 235(3):959-73 (1994), each of which is incorporated herein in its entirety by reference. Often, framework residues in the framework regions will be substituted with the corresponding residue from the CDR donor antibody to alter, for example improve, antigen binding. These framework substitutions are identified by methods well-known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature, 332:323, which are incorporated herein by reference in their entireties.)

A humanized antibody or antibody fragment has one or more amino acid residues remaining in it from a source which is non-human. These non-human amino acid residues are often referred to as “import” residues, which are typically taken from an “import” variable domain. As provided herein, humanized antibodies or antibody fragments comprise one or more CDRs from non-human immunoglobulin molecules and framework regions wherein the amino acid residues comprising the framework are derived completely or mostly from human germline. Multiple techniques for humanization of antibodies or antibody fragments are well-known in the art and can essentially be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody, i.e., CDR-grafting (EP 239,400; PCT Publication No. WO 91/09967; and U.S. Pat. Nos. 4,816,567; 6,331,415; 5,225,539; 5,530,101; 5,585,089; 6,548,640, the contents of which are incorporated herein by reference herein in their entirety). In such humanized antibodies and antibody fragments, substantially less than an intact human variable domain has been substituted by the corresponding sequence from a non-human species. Humanized antibodies are often human antibodies in which some CDR residues and possibly some framework (FR) residues are substituted by residues from analogous sites in rodent antibodies. Humanization of antibodies and antibody fragments can also be achieved by veneering or resurfacing (EP 592,106; EP 519,596; Padlan, 1991, Molecular Immunology, 28(4/5):489-498; Studnicka et al., Protein Engineering, 7(6):805-814 (1994); and Roguska et al., PNAS, 91:969-973 (1994)) or chain shuffling (U.S. Pat. No. 5,565,332), the contents of which are incorporated herein by reference herein in their entirety.

In some embodiments, an antigen binding domain is derived from a display library. A display library is a collection of entities; each entity includes an accessible polypeptide component and a recoverable component that encodes or identifies the polypeptide component. The polypeptide component is varied so that different amino acid sequences are represented. The polypeptide component can be of any length, e.g., from three amino acids to over 300 amino acids. A display library entity can include more than one polypeptide component, for example, the two polypeptide chains of a Fab. In one exemplary embodiment, a display library can be used to identify an antigen binding domain. In a selection, the polypeptide component of each member of the library is probed with the antigen, or a fragment there, and if the polypeptide component binds to the antigen, the display library member is identified, typically by retention on a support.

Retained display library members are recovered from the support and analyzed. The analysis can include amplification and a subsequent selection under similar or dissimilar conditions. For example, positive and negative selections can be alternated. The analysis can also include determining the amino acid sequence of the polypeptide component and purification of the polypeptide component for detailed characterization.

A variety of formats can be used for display libraries. Examples include the phage display. In phage display, the protein component is typically covalently linked to a bacteriophage coat protein. The linkage results from translation of a nucleic acid encoding the protein component fused to the coat protein. The linkage can include a flexible peptide linker, a protease site, or an amino acid incorporated as a result of suppression of a stop codon. Phage display is described, for example, in U.S. Pat. No. 5,223,409; WO 92/18619; WO 91/17271; WO 92/20791; WO 92/15679; WO 93/01288; WO 92/01047; WO 92/09690; WO 90/02809. Bacteriophage displaying the protein component can be grown and harvested using standard phage preparatory methods, e.g. PEG precipitation from growth media. After selection of individual display phages, the nucleic acid encoding the selected protein components can be isolated from cells infected with the selected phages or from the phage themselves, after amplification. Individual colonies or plaques can be picked, the nucleic acid isolated and sequenced. Other display formats include cell based display (see, e.g., WO 03/029456), protein-nucleic acid fusions (see, e.g., U.S. Pat. No. 6,207,446), ribosome display, and E. coli periplasmic display.

In some embodiments, the antigen binding domain specifically binds a cancer-specific antigen. Non-limiting examples of cancer-specific antigens that may be bound by the antigen binding domain of the CAR described herein include: CD19, BCMA, TACI, CD79b, CD22, CD30, CS1, GPCR, PSMA, mesothelin, MUC1, MUC16, EGFR, IL-13Ralpha2, EGFRvIII, CD20, CD79a, or combinations thereof.

In some embodiments, the antigen binding domain binds to a MHC presented peptide. Normally, peptides derived from endogenous proteins fill the pockets of Major histocompatibility complex (MHC) class I molecules, and are recognized by T cell receptors (TCRs) on CD8+ T lymphocytes. The MHC class I complexes are constitutively expressed by all nucleated cells. In cancer, virus-specific and/or tumor-specific peptide/MHC complexes represent a unique class of cell surface targets for immunotherapy. TCR-like antibodies targeting peptides derived from viral or tumor antigens in the context of human leukocyte antigen (HLA)-A1 or HLA-A2 have been described (see, e.g., Maus et al, Mol Ther Oncolytics. 2017 Jan. 11; 3:1-9; Sastry et al., J Virol. 2011 85(5):1935-1942; Sergeeva et al., Blood, 2011 117(16):4262-4272; Verma et al., J Immunol 2010 184(4):2156-2165; Willemsen et al., Gene Ther 2001 8(21):1601-1608; Dao et al., Sci Transl Med 2013 5(176):176ra33; Tassev et al., Cancer Gene Ther 2012 19(2):84-100). For example, TCR-like antibody can be identified from screening a library, such as a human scFv phage displayed library.

In some embodiments, the CARs described herein further comprises a leader sequence at the amino-terminus (N-terminus) of the antigen binding domain. In some embodiments, the CAR further comprises a leader sequence at the N-terminus of the antigen binding domain, wherein the leader sequence is optionally cleaved from the antigen binding domain (e.g., a scFv) during cellular processing and localization of the CAR to the cellular membrane. In some embodiments, the leader sequence is an interleukin 2 signal peptide or a CD8 leader sequence. In some embodiments, the leader sequence comprises an amino acid sequence of: MALPVTALLLPLALLLHAARP (SEQ ID NO: 15).

The transmembrane domain of the CARs described herein may be derived either from a natural or from a recombinant source. Where the source is natural, the domain may be derived from any membrane-bound or transmembrane protein. In one aspect the transmembrane domain is capable of signaling to the intracellular domain(s) whenever the CAR has bound to a target. A transmembrane domain of particular use in this invention may include at least the transmembrane region(s) of e.g., the alpha, beta or zeta chain of the T-cell receptor, CD28, CD3 epsilon, CD45, CD4, CD5, CD8 (e.g., CD8 alpha, CD8 beta), CD9, CD16, CD22, CD33, CD37, CD64, CD80, CD86, CD134, CD137, CD154. In some embodiments, a transmembrane domain may include at least the transmembrane region(s) of, e.g., KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2R beta, IL2R gamma, IL7R a, ITGA1, VLA1, CD49a, ITGA4, IA4, CD49D, ITGA6, VLA-6, CD49f, ITGAD, CD11d, ITGAE, CD103, rfGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), SLAMF6 (NTB-A, Lyl08), SLAM (SLAMF1, CD150, IPO-3), BLAME (SLAMF8), SELPLG (CD162), LTBR, PAG/Cbp, NKG2D, NKG2C, and CD19. In some embodiments, the transmembrane domain is a CD28 transmembrane domain or CD8 transmembrane domain.

In some embodiments, the transmembrane domain can be attached to the extracellular region of the CAR, e.g., the ligand domain of the CAR, via a hinge, e.g., a hinge from a human protein. For example, in one embodiment, the hinge can be a human Ig (immunoglobulin) hinge, e.g., an IgG4 hinge, or a CD8a hinge.

The cytoplasmic domain or region of the present CAR includes one or more intracellular signaling domains. An intracellular signaling domain is capable of activation of at least one of the normal effector functions of the immune cell in which the CAR has been introduced. Examples of intracellular signaling domains for use in the CAR described herein include the cytoplasmic sequences of the T cell receptor (TCR) and co-receptors that act in concert to initiate signal transduction following antigen receptor engagement, as well as any derivative or variant of these sequences and any recombinant sequence that has the same functional capability.

T cell activation can be said to be mediated by two distinct classes of cytoplasmic signaling sequences: those that initiate antigen-dependent primary activation through the TCR (primary intracellular signaling domains) and those that act in an antigen-independent manner to provide a secondary or costimulatory signal (secondary cytoplasmic domain, e.g., a costimulatory domain).

An “intracellular signaling domain,” as the term is used herein, refers to an intracellular portion of a molecule. The intracellular signaling domain can generate a signal that promotes an immune effector function of the CAR containing cell, e.g., a CAR T cell or CAR-expressing NK cell. Examples of immune effector function, e.g., in a CAR T cell or CAR-expressing NK cell, include cytolytic activity and helper activity, including the secretion of cytokines. In embodiments, the intracellular signal domain transduces the effector function signal and directs the cell to perform a specialized function. While the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

In some embodiments, the one or more intracellular signaling domains comprise a primary intracellular signaling domain. Exemplary primary intracellular signaling domains include those derived from the molecules responsible for primary stimulation, or antigen dependent simulation. In some embodiments, a primary intracellular signaling domain comprises a signaling motif which is known as an immunoreceptor tyrosine-based activation motif or ITAM. Examples of ITAM containing primary cytoplasmic signaling sequences include, but are not limited to, those derived from CD3 zeta, FcR gamma, FcR beta, CD3 gamma, CD3 delta, CD3 epsilon, CD3 theta, CD3 eta, CD5, CD22, CD79a, CD79b, CD278 (“ICOS”), FceRI, CD66d, DAP10, and DAP12. In some embodiments, the intracellular signaling domain of the CAR comprises a CD3-zeta (CD3ζ) signaling domain.

In some embodiments, the one or more intracellular signaling domain comprise a costimulatory intracellular domain. A costimulatory intracellular signaling domain refers to the intracellular portion of a costimulatory molecule. The intracellular signaling domain can comprise the entire intracellular portion, or the entire native intracellular signaling domain, of the molecule from which it is derived, or a functional fragment thereof. Exemplary costimulatory intracellular signaling domains include those derived from molecules responsible for costimulatory signals (e.g., antigen independent stimulation), and those derived from cytokine receptors. In some embodiments, the one or more intracellular signaling domains comprise a primary intracellular signaling domain, and a costimulatory intracellular signaling domain from one or more co-stimulatory proteins or cytokine receptors.

The term “costimulatory molecule” refers to the cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are required for an efficient immune response. Examples of such molecules include a MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD28, CD30, CD40, CDS, ICAM-1, LFA-1 (CD1 1a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRFI), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11 d, ITGAE, CD103, ITGAL, CD11a, LFA-1, ITGAM, CD11b, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CDIOO (SEMA4D), CD69, SLAMF6 (NTB-A, Lyl08), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83. For example, CD27 co-stimulation has been demonstrated to enhance expansion, effector function, and survival of human CART cells in vitro and augments human T cell persistence and antitumor activity in vivo (Song et al. Blood. 2012; 119(3):696-706). In some embodiments, the co-stimulatory domain of the CARs described herein comprises on or more signaling domains from one or more co-stimulatory protein or cytokine receptor selected from CD28, 4-1BB, 2B4, KIR, CD27, OX40, ICOS, MYD88, IL2 receptor, and SynNotch.

In some embodiments, the intracellular signalling domain of the CAR described herein comprise the primary signalling domain, e.g., an ITAM containing domain such as a CD3-zeta signaling domain, by itself or combined with a costimulatory signaling domain (e.g., a co-stimulating domain from one or more co-stimulatory protein or cytokine receptor selected from CD28, 4-1BB, 2B4, KIR, CD27, OX40, ICOS, MYD88, IL2 receptor, and SynNotch). In some embodiments, the intracellular signalling domain of the CAR described herein comprise a CD3-zeta signaling domain and a costimulatory signaling domain such as the costimulatory signaling domain from CD28 or 4-1BB.

In some embodiments, the engineered immune cell described herein comprises an engineered T cell receptor (TCR), with or without the inactivation of the endogenous TCR. In some embodiments, the TCR is engineered to bind specific antigens that an endogenous has low binding affinity to. For example, the specific antigen may be a tumor antigen.

To generate any one of the engineered immune cells that comprises a CAR or TCR and deficient in IFNγ described herein, a nucleic acid comprising a nucleotide sequence encoding the CAR or the engineered TCR operably linked to a promoter may be delivered to the immune cell, and/or any one of the nucleotide sequences or methods that suppresses the IFNγ gene described herein may be used. In some embodiments, the engineered immune cells comprises a nucleic acid comprising a nucleotide sequence encoding the CAR or the engineered TCR operably linked to a promoter and a nucleotide sequence that suppresses the IFNγ gene (e.g., a gRNA or a RNAi molecule that target the IFNγ gene).

In some aspects, the disclosure provides nucleic acid molecules (e.g., vectors) for expressing CARs or TCRs in cells, e.g., T cells. In some embodiments, the nucleic acid molecule comprises a nucleotide sequence encoding the CAR or TCR. The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Recombinant DNA and molecular cloning techniques used here are well known in the art and are described, for example, by Sambrook, J., Fritsch, E. F. and Maniatis, T. MOLECULAR CLONING: A LABORATORY MANUAL, 2nd ed.; Cold Spring Harbor Laboratory: Cold Spring Harbor, N.Y., 1989; and by Silhavy, T. J., Bennan, M. L. and Enquist, L. W. EXPERIMENTS WITH GENE FUSIONS; Cold Spring Harbor Laboratory: Cold Spring Harbor, N.Y., 1984; and by Ausubel, F. M. et al., IN CURRENT PROTOCOLS IN MOLECULAR BIOLOGY, published by Greene Publishing and Wiley-Interscience, 1987; (the entirety of each of which is hereby incorporated herein by reference). Alternatively, the gene of interest can be produced synthetically, rather than cloned.

In some embodiments, the desired CAR or engineered TCR can be expressed in the cells by way of transposons. In some embodiments, expression of natural or synthetic nucleic acids CARs or engineered TCR is typically achieved by operably linking a nucleic acid encoding the CAR or engineered TCR to a promoter, and incorporating the construct into an expression vector. The vectors can be suitable for replication and integration into eukaryotes. Typical cloning vectors contain transcription and translation terminators, initiation sequences, and promoters useful for regulation of the expression of the desired nucleic acid sequence. The expression constructs of the disclosure may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Factor-1a (EF-1a). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the disclosure is not limited to the use of constitutive promoters. Inducible promoters are also contemplated as part of the disclosure. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter. In some embodiments, the promoter is a EF-1a promoter.

In some embodiments, the nucleic acid molecule comprising a nucleotide sequence encoding the CAR or TCR described herein is a vector. The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, retrovirus vectors are used. A number of retrovirus vectors are known in the art. In some embodiments, lentivirus vectors are used.

Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. A “lentivirus” as used herein refers to a genus of the Retroviridae family. Lenti viruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lenti viruses. Vectors derived from lenti viruses offer the means to achieve significant levels of gene transfer in vivo.

In some embodiments, the engineered immune cell described herein comprises a first nucleotide sequence encoding a CAR or engineered TCR and a second nucleotide sequence encoding an agent that suppresses the IFNγ gene (e.g., any one of the nucleotide sequences or nucleases that suppresses the IFNγ gene provided herein or known in the art). In some embodiments, the first nucleotide sequence and the second nucleotide sequence are on the same nucleic acid molecule (e.g., a vector such as a lentiviral vector or retroviral vector described herein).

As such, some aspects of the present disclosure provide nucleic acid molecules (e.g., vectors such as lentiviral vectors or retroviral vectors) comprising: (i) a first nucleotide sequence encoding a CAR or an engineered TCR (e.g., any one of the CAR or engineered TCR described herein); and (ii) a second nucleotide sequence encoding an agent that suppresses the IFNγ gene (e.g., any one of the gRNAs, ribozymes, RNAi molecules (e.g., a siRNA, a miRNA, a shRNA, an antisense oligonucleotide), or nucleotide sequences encoding nucleases (e.g., a TALEN, a ZFN, or a meganuclease) targeting the IFNγ gene). In some embodiments, the nucleic acid molecules (e.g., vectors such as lentiviral vectors or retroviral vectors) further comprise a third nucleotide sequence encoding an agent that suppresses an endogenous T cell receptor (TCR) gene (e.g., any one of the gRNAs, ribozymes, RNAi molecules (e.g., a siRNA, a miRNA, a shRNA, an antisense oligonucleotide), or nucleotide sequences encoding nucleases (e.g., a TALEN, a ZFN, or a meganuclease) targeting the TCR gene). In some embodiments, the endogenous TCR gene is T Cell Receptor Alpha Constant (TRAC) or T Cell Receptor Beta Constant (TRBC). In some embodiments, the endogenous TCR gene is any gene encoding a component of the CD3 complex (e.g., CD3γ, CD3δ, and CD3ε). CD3 (cluster of differentiation 3) is a protein complex and T cell co-receptor that is involved in activating both the cytotoxic T cell (CD8+ naive T cells) and T helper cells (CD4+ naive T cells).

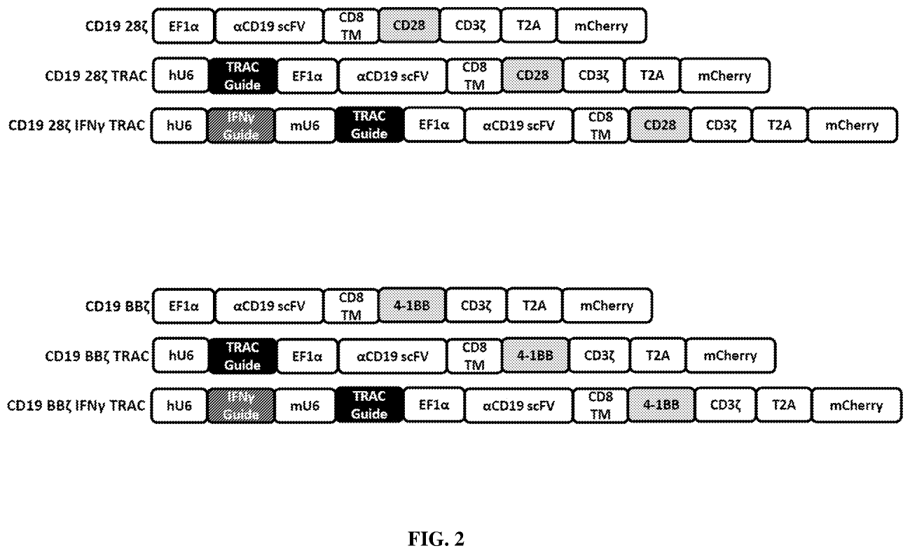

In some embodiments, the nucleic acid molecule (e.g., a vector such as a lentiviral vector or a retroviral vector) comprises: i) a first nucleotide sequence encoding a CAR or an engineered TCR (e.g., any one of the CAR or engineered TCR described herein); and (ii) a second nucleotide sequence encoding a gRNA that targets the IFNγ gene (e.g., the gRNA comprising the nucleotide sequence of CCAGAGCATCCAAAAGAGTG (SEQ ID NO: 1) or TGAAGTAAAAGGAGACAATT (SEQ ID NO: 2)). In some embodiments, the nucleic acid molecule (e.g., a vector such as a lentiviral vector or a retroviral vector) comprises: (i) a first nucleotide sequence encoding a CAR or an engineered TCR (e.g., any one of the CAR or engineered TCR described herein); (ii) a second nucleotide sequence encoding a gRNA that targets the IFNγ gene (e.g., the gRNA comprising the nucleotide sequence of CCAGAGCATCCAAAAGAGTG (SEQ ID NO: 1) or TGAAGTAAAAGGAGACAATT (SEQ ID NO: 2)); and (iii) a third nucleotide sequence encoding a gRNA that targets an endogenous TCR gene (e.g., TRAC, TRBC, or a CD3 complex component). Non-limiting examples of such nucleic acid molecules are illustrated in FIG. 2. It is to be understood that the sequences and schematics of the CAR constructs are for illustration purpose only and are not meant to be limiting. The engineered immune cells described herein may be engineered to express any CAR.

Any one of the nucleic acid molecules described herein may be used to produce the engineered immune cell described herein, e.g., by delivering the nucleic acid molecule into the immune cell and culturing the cell under conditions that allow the expression of the CAR or TCR and the agent that suppresses the IFNγ gene (e.g., a gRNA). In some embodiments, when the agent that suppresses the IFNγ gene is a gRNA, the method further comprises delivering to the immune cell a nucleic acid comprising a nucleotide sequence encoding a Cas9 nuclease (e.g., a mRNA encoding the Cas9 nuclease) or delivering to the immune cell an isolated Cas9 nuclease.

Any methods known in the art for delivering nucleic acids or proteins into a cell may be used, e.g., transfection, transformation, transduction, or electroporation. The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

In some embodiments, the immune cell is a mammalian immune cell. In some embodiments, the immune cell is a human immune cell. In some embodiments, the immune cell is a human T cell.

Immune cells (e.g., T cells) can be obtained from a number of sources, including peripheral blood mononuclear cells, bone marrow, lymph node tissue, cord blood, thymus tissue, tissue from a site of infection, ascites, pleural effusion, spleen tissue, and tumors. The immune cells (e.g., T cells) may also be generated from induced pluripotent stem cells or hematopoietic stem cells or progenitor cells. In some embodiments, any number of immune cell lines, including but not limited to T cell lines, including, for example, Hep-2, Jurkat, and Raji cell lines, available in the art, may be used. In some embodiments, immune cells (e.g., T cells) can be obtained from a unit of blood collected from a subject using any number of techniques known to the skilled artisan, such as Ficoll™ separation. In some embodiments, cells from the circulating blood of an individual are obtained by apheresis. The apheresis product typically contains lymphocytes, including T cells, monocytes, granulocytes, B cells, NK cells, other nucleated white blood cells, red blood cells, and platelets. In some embodiments, the cells collected by apheresis may be washed to remove the plasma fraction and to place the cells in an appropriate buffer or media for subsequent processing steps. In some embodiments, the cells are washed with phosphate buffered saline (PBS). In some embodiments, the wash solution lacks calcium and may lack magnesium or may lack many if not all divalent cations. Again, surprisingly, initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca²⁺-free, Mg²⁺-free PBS, PlasmaLyte A, or other saline solution with or without buffer. Alternatively, the undesirable components of the apheresis sample may be removed and the cells directly resuspended in culture media.

In some embodiments, immune cells (e.g., T cells) are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3⁺, CD28⁺, CD4⁺, CD8⁺, CD45RA⁺, and CD45RO⁺T cells, can be further isolated by positive or negative selection techniques.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD11b, CD16, HLA-DR, and CD8. In certain embodiments, it may be desirable to enrich for or positively select for regulatory T cells which typically express CD4⁺, CD25⁺, CD62L^{hi}, GITR⁺, and FoxP3⁺. Alternatively, in some embodiments, T regulatory cells are depleted by anti-C25 conjugated beads or other similar method of selection.

Other aspects of the present disclosure provide compositions comprising any one of the engineered immune cells described herein. In some embodiments, the composition is a pharmaceutical composition. In some embodiments, the composition further comprises a pharmaceutically acceptable carrier, excipients or stabilizers typically employed in the art (all of which are termed “excipients”), for example buffering agents, stabilizing agents, preservatives, isotonifiers, non-ionic detergents, antioxidants and/or other miscellaneous additives.

In some embodiments, any one of the engineered immune cells described herein or any one of the compositions comprising the engineered immune cells described herein is administered to a subject. Accordingly, some aspects of the present disclosure provide methods of administering to a subject any one of the engineered immune cells or the compositions comprising the engineered immune cells described herein. In some embodiments, the method is for treating cancer or an autoimmune disease, and the method comprises administering to a subject in need thereof an effective amount of the engineered immune cells or the compositions comprising the engineered immune cells described herein. In some embodiments, the method is for reducing cytokine release associated with CAR T cell therapy, the method comprising administering to a subject in need thereof an effective amount of the engineered immune cells or the compositions comprising the engineered immune cells described herein.

To practice the methods described herein, an effective amount of the engineered immune cells may be administered to a subject via a suitable route (e.g., intravenous infusion). The immune cell population may be mixed with a pharmaceutically acceptable carrier to form a pharmaceutical composition prior to administration, which is also within the scope of the present disclosure. The engineered immune cells may be autologous to the subject, i.e., the immune cells are obtained from the subject in need of the treatment, modified to reduce expression of one or more target cytokines/proteins, for example, those described herein, to express one or more cytokine antagonists described herein, to express a CAR construct and/or exogenous TCR, or a combination thereof. The resultant modified immune cells can then be administered to the same subject. Administration of autologous cells to a subject may result in reduced rejection of the immune cells as compared to administration of non-autologous cells. Alternatively, the engineered immune cells can be allogenic cells, i.e., the cells are obtained from a first subject, modified as described herein and administered to a second subject that is different from the first subject but of the same species. For example, allogenic immune cells may be derived from a human donor and administered to a human recipient who is different from the donor.

The subject to be treated may be a mammal (e.g., human, mouse, pig, cow, rat, dog, guinea pig, rabbit, hamster, cat, goat, sheep or monkey). The subject may be suffering from cancer or an immune disorder (e.g., an autoimmune disease).

Exemplary cancers include, without limitation: Oral: buccal cavity, lip, tongue, mouth, pharynx; Cardiac: sarcoma (angiosarcoma, fibrosarcoma, rhabdomyosarcoma, liposarcoma), myxoma, rhabdomyoma, fibroma, lipoma and teratoma; Lung: non-small cell lung cancer (NSCLC), small cell lung cancer, bronchogenic carcinoma (squamous cell or epidermoid, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, larynx, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel or small intestines (adenocarcinoma, lymphoma, carcinoid tumors, Karposi's sarcoma, leiomyoma, hemangioma, lipoma, neurofibroma, fibroma), large bowel or large intestines (adenocarcinoma, tubular adenoma, villous adenoma, hamartoma, leiomyoma), rectal, colon, colon-rectum, colorectal; Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma, biliary passages; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), head and neck cancer, meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, SertolI-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma), breast; Hematologic: blood (myeloid leukemia [acute and chronic], acute lymphoblastic leukemia, myeloproliferative diseases, multiple myeloma, myelodysplastic syndrome), Hodgkin's disease, non-Hodgkin's lymphoma [malignant lymphoma] hairy cell; lymphoid disorders; Skin: malignant melanoma, basal cell carcinoma, squamous cell carcinoma, Karposi's sarcoma, keratoacanthoma, moles dysplastic nevi, lipoma, angioma, dermatofibroma, keloids, psoriasis, Thyroid gland: papillary thyroid carcinoma, follicular thyroid carcinoma; medullary thyroid carcinoma, multiple endocrine neoplasia type 2A, multiple endocrine neoplasia type 2B, familial medullary thyroid cancer, pheochromocytoma, paraganglioma; and Adrenal glands: neuroblastoma. In some embodiments, the cancer treated using any one of the engineered immune cells or methods described herein is a liquid tumor, e.g., without limitation, lymphomas and leukemias. In some embodiments, the cancer treated using any one of the engineered immune cells or methods described herein is a liquid tumor, e.g., without limitation, mantle cell lymphoma and acute lymphoblastic leukemia.

Exemplary autoimmune diseases include, without limitation, rheumatoid arthritis, type I diabetes, systemic lupus erythematosus, inflammatory bowel disease, multiple sclerosis, Guillain-Barre syndrome, chronic inflammatory demyelinating polyneuropathy, psoriasis, Graves' disease, Hashimoto's thyroiditis, myasthenia gravis, and vasculitis.

The term “an effective amount” as used herein refers to the amount of each active agent required to confer therapeutic effect on the subject, either alone or in combination with one or more active agents. Effective amounts vary, as recognized by those skilled in the art, depending on the particular condition being treated, the severity of the condition, individual patient parameters including age, physical condition, size, gender and weight, the duration of treatment, route of administration, excipient usage, co-usage (if any) with other active agents and like factors within the knowledge and expertise of the health practitioner. The quantity to be administered depends on the subject to be treated, including, for example, the capacity of the individual's immune system to produce a cell-mediated immune response. Precise mounts of active ingredient required to be administered depend on the judgment of the practitioner. However, suitable dosage ranges are readily determinable by one skilled in the art.

The term “treating” as used herein refers to the application or administration of a composition including one or more active agents to a subject, who has a target disease, a symptom of the target disease, or a predisposition toward the target disease, with the purpose to cure, heal, alleviate, relieve, alter, remedy, ameliorate, improve, or affect the disease, the symptoms of the disease, or the predisposition toward the disease.

The therapeutic methods described herein may be utilized in conjunction with other types of therapy for cancer, such as chemotherapy, surgery, radiation, gene therapy, and so forth. Such therapies can be administered simultaneously or sequentially (in any order) with the immunotherapy described herein. When co-administered with an additional therapeutic agent, suitable therapeutically effective dosages for each agent may be lowered due to the additive action or synergy.

Non-limiting examples of other anti-cancer therapeutic agents useful for combination with the modified immune cells described herein include, but are not limited to, immune checkpoint inhibitors (e.g., PDL1, PD1, and CTLA4 inhibitors), anti-angiogenic agents (e.g., TNP-470, platelet factor 4, thrombospondin-1, tissue inhibitors of metalloproteases, prolactin, angiostatin, endostatin, bFGF soluble receptor, transforming growth factor beta, interferon alpha, soluble KDR and FLT-1 receptors, and placental proliferin-related protein); a VEGF antagonist (e.g., anti-VEGF antibodies, VEGF variants, soluble VEGF receptor fragments); chemotherapeutic compounds. Exemplary chemotherapeutic compounds include pyrimidine analogs (e.g., 5-fluorouracil, floxuridine, capecitabine, gemcitabine and cytarabine); purine analogs (e.g., fludarabine); folate antagonists (e.g., mercaptopurine and thioguanine); antiproliferative or antimitotic agents, for example, vinca alkaloids; microtubule disruptors such as taxane (e.g., paclitaxel, docetaxel), vincristin, vinblastin, nocodazole, epothilones and navelbine, and epidipodophyllotoxins; DNA damaging agents (e.g., actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethyhnelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide).

In some embodiments, radiation, or radiation and chemotherapy are used in combination with the cell populations comprising modified immune cells described herein. Additional useful agents and therapies can be found in Physician's Desk Reference, 59.sup.th edition, (2005), Thomson P D R, Montvale N.J.; Gennaro et al., Eds. Remington's The Science and Practice of Pharmacy 20.sup.th edition, (2000), Lippincott Williams and Wilkins, Baltimore Md.; Braunwald et al., Eds. Harrison's Principles of Internal Medicine, 15.sup.th edition, (2001), McGraw Hill, N.Y.; Berkow et al., Eds. The Merck Manual of Diagnosis and Therapy, (1992), Merck Research Laboratories, Rahway N.J.

The engineered immune cells described herein can be administered via conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In addition, it can be administered to the subject via injectable depot routes of administration such as using 1-, 3-, or 6-month depot injectable or biodegradable materials and methods. In some embodiments, the pharmaceutical composition is administered intraocularly or intravitreally.

Due to the deficiency of IFNγ expression by the engineered immune cells described herein, the therapeutic uses of such cells would be expected to reduce cytotoxicity (e.g., reduced cytokine release) associated with conventional immune cell therapy (reducing inflammatory cytokine production and/or signaling by both the immune cells used in adoptive immune cell therapy and endogenous immune cells of the recipient, which can be activated by the infused immune cells), while achieving the same or better therapeutic effects.

In some embodiments, the level of inflammatory cytokines, chemokines, and/or adhesion molecules produced in the subject administered the engineered immune cells described herein are reduced (e.g., by at least 10%, at least 20%, at least 30%, at least 40%, at least 50%, at least 60%, at least 70%, at least 80%, at least 90%, at least 95%, at least 99%, or more), compared to that of a subject administered an engineered immune cell not deficient in IFNγ expression. In some embodiments, the inflammatory cytokines, the chemokines, or the adhesion molecules are selected from: IL-4, IL-10, IL-12, IL-13, MIP1α, MIP1β, MCP1, IP10, E-selectin, P-selection, PSEL, IL-1beta, IL12p70 and SICAM1. In some embodiments, the reduction of the level of the inflammatory cytokines, chemokines, and/or adhesion molecules is in cancer microenvironment, circulation or central nervous system. “Cancer microenvironment” refers to the environment around a tumor, including the surrounding blood vessels, immune cells (e.g., macrophages), fibroblasts, signaling molecules and the extracellular matrix (ECM). The cancer and the surrounding microenvironment are closely related and interact constantly. Cancers can influence the microenvironment by releasing extracellular signals, promoting cancer angiogenesis and inducing peripheral immune tolerance, while the immune cells (e.g., macrophages) in the microenvironment can affect the growth and evolution of cancerous cells.

The present disclosure also provides kits for use in any of the target diseases described herein (e.g., cancer or autoimmune disease) involving the engineered immune cells described herein and kits for use in making the engineered immune cells as described herein.

A kit for therapeutic use as described herein may include one or more containers comprising the engineered immune cell, which may be formulated to form a pharmaceutical composition. The engineered immune cells, such as T lymphocytes, NK cells, and others described herein may further express a CAR construct and/or an exogenous TCR, as described herein.

In some embodiments, the kit can additionally comprise instructions for use of the engineered immune cells in any of the methods described herein. The included instructions may comprise a description of administration of the immune cell population or a pharmaceutical composition comprising such to a subject to achieve the intended activity in a subject. The kit may further comprise a description of selecting a subject suitable for treatment based on identifying whether the subject is in need of the treatment. In some embodiments, the instructions comprise a description of administering the immune cell population or the pharmaceutical composition comprising such to a subject who is in need of the treatment.

The instructions relating to the use of the engineered immune cells or the pharmaceutical composition comprising such cells as described herein generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

The kits provided herein are in suitable packaging. Suitable packaging includes, but is not limited to, vials, bottles, jars, flexible packaging, and the like. Also contemplated are packages for use in combination with a specific device, such as an inhaler, nasal administration device, or an infusion device. A kit may have a sterile access port (for example, the container may be an intravenous solution bag or a vial having a stopper pierceable by a hypodermic injection needle). The container may also have a sterile access port. At least one active agent in the pharmaceutical composition is a population of immune cells (e.g., T lymphocytes or NK cells) that comprise any of the modified immune cells or a combination thereof.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

Also provided herein are kits for use in making the engineered immune cells as described herein. Such a kit may include one or more containers each containing reagents for use in introducing nucleic acid molecules (e.g., vectors, mRNAs, RNAi molecules) or isolated proteins (e.g., isolated Cas9, TALEN, or ZFN) into immune cells. For example, the kit may contain one or more components of a gene editing system for making one or more gene modifications as those described herein. Such a kit may further include instructions for making the desired modifications to host immune cells.

### EXAMPLES

### Example 1—IFNγ Blockade does not Reduce CAR-T Activation or Cytotoxicity In Vitro

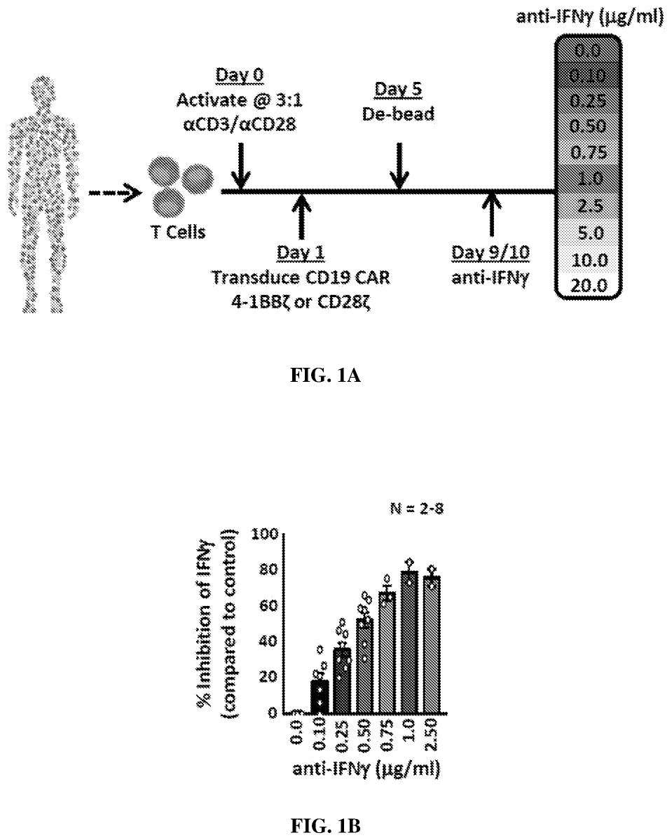

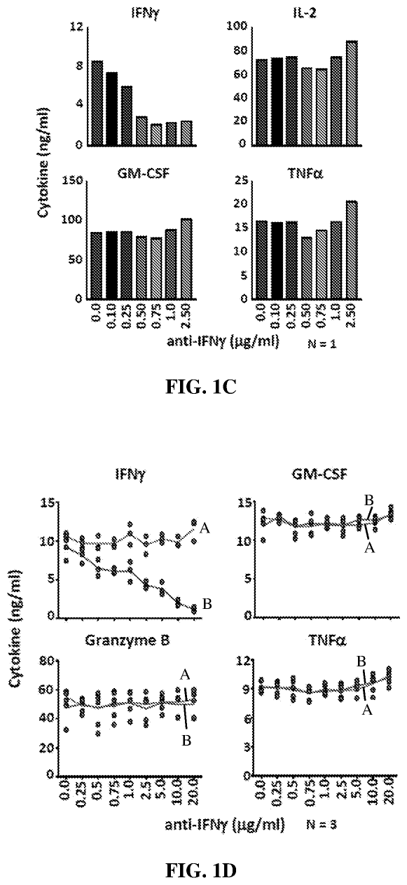

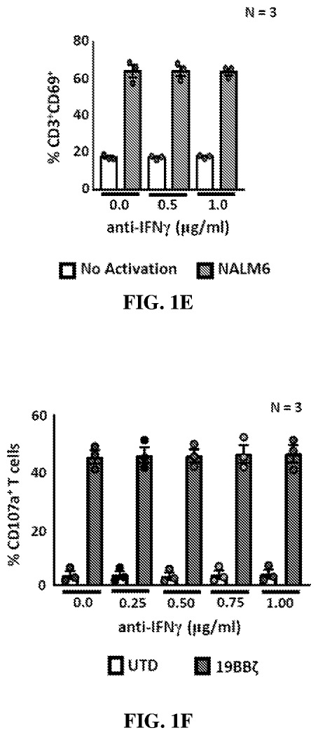

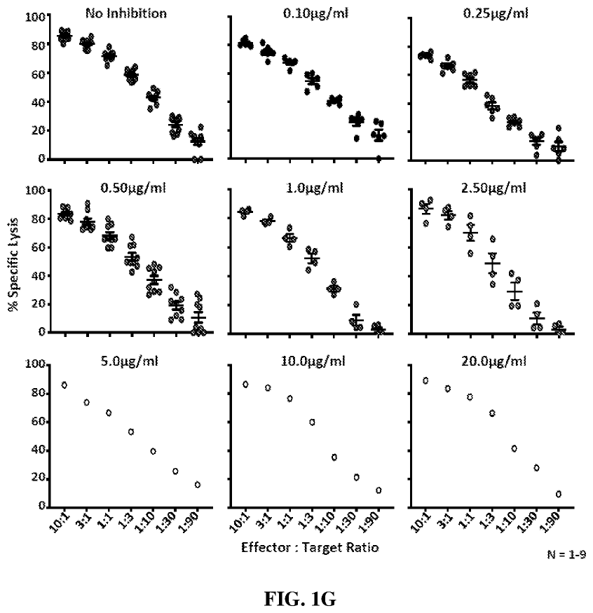

T cells isolated from healthy donors were stimulated with beads coated in CD3 and CD28 antibodies for 24 hours before transducing with a lentiviral vector to express either a CD19-41BBζ or CD19-CD28ζ chimeric antigen receptor (CAR). On Day 5, the stimulation beads were removed. On Day 9 or 10, the cells were treated with various doses of anti-IFNγ antibody (0 to 20 μg/mL) for one hour (FIG. 1A). Following anti-IFNγ treatment, the CAR T cells were activated by mixing with NALM6 (CD19-expressing leukemia) cells at a ratio of 1:1. After 6 hours, IFNγ production was measured by ELISA and compared to control cells not treated with anti-IFNγ. Incubation with anti-IFNγ inhibited IFNγ production by CAR T cells in a dose-dependent manner, up to around an 80% inhibition relative to control CAR T cells. To test the effect of IFNγ blockade on the production of other cytokines by CAR T cells, cells were activated with PMA and ionomycin for 6 hours following anti-IFNγ treatment. Production of IFNγ, IL-2, GM-CSF, and TNFα was measured by ELISA. Antibody treatment did not substantially affect IL-2, GM-CSF, or TNFα production by CAR T cells at any doses tested, including those which showed substantial decreases in IFNγ production (FIG. 1C). This suggested that IFNγ blockade does not affect CAR T cell activation. To test the effects on a longer time scale, following anti-IFNγ treatment, CAR T cells were activated by mixing with NALM6 (CD19 expressing leukemia) cells at a ratio of 1:1. After 18 hours, production of IFNγ, GM-CSF, granzyme B and TNFα was measured by Luminex. Antibody treatment did not affect GM-CSF, granzyme B, or TNFα production by CAR T cells at any doses tested, up to 20 μg/mL (FIG. 1D), again suggesting that IFNγ blockade does not affect CAR T cell activation. Six hours after activation with NALM6 cells, CD69 expression and CD107a expression on CD3+ CAR T cells treated with anti-IFNγ at various concentrations were measured by flow cytometry. Antibody treatment did not affect the expression of either marker at any dose tested (FIGS. 1E-1F). To test the effect of IFNγ blockade on CAR T cell mediated cytotoxicity, CAR T cells were mixed with NALM6 cells at varying effector to target cells ratios (E:T) for 18 hours following treatment with anti-IFNγ at varying concentrations (0 to 20 g/mL), after which cell specific lysis was determined using a luciferase assay. IFNγ blockade did not affect CAR T cell mediated lysis of NALM6 cells at any concentration tested (FIG. 1G). These data demonstrate that, unexpectedly, locking IFNγ using an anti-IFNγ antibody reduces IFNγ production by CAR T cells but does not affect their overall functionality or ability to become activated, as evidenced by their ability to continue producing other cytokines and killing leukemia cells when activated.

### Lentiviral Construct Design

To facilitate stable depletion of IFNγ in engineered CAR T cells, lentiviral constructs were designed to express CD19 CARs concurrently with sgRNA (guides) targeting IFNγ. Additional constructs were designed to express CD19 CARs concurrently with sgRNA (guides) targeting the endogenous T cell receptor (TRAC) or sgRNAs targeting both TRAC and IFNγ. Each of these constructs were compared to additional constructs expressing CD19 CARs without sgRNAs. CD19 CARs have an extracellular domain that will bind to CD19 on target cells (anti-CD19 scFV) and have either CD28 or 41BBζ combined with CD3ζ intracellular signaling domains. All constructs contain mCherry as a way to identify cells that have been transduced with the vector (FIG. 2). Exemplary sequences of the components of a CAR encoded by the constructs described herein is provided in Table 1 below. It is to be understood that the sequences and schematics of the CAR constructs are for illustration purpose only and are not meant to be limiting. The engineered immune cells described herein may be engineered to express any CAR.

TABLE 1
Examples of sequences in the constructs shown in FIG. 2

| | |
|---|---|
| gRNA targeting TRAC | AGAGTCTCTCAGCTGGTACAGTTTTAGAGCTAGAAAT |
| (optional, guide sequence | AGCAAGTTAAAATAAGGCTAGTCCGTTATCAACTTGA |
| underlined) | AAAAGTGGCACCGAGTCGGTGCTTTTTT (SEQ ID NO: |
| | 4) |
| gRNA targeting IFNγ | CCAGAGCATCCAAAAGAGTGGTTTTAGAGCTAGAAA |
| (guide sequence | TAGCAAGTTAAAATAAGGCTAGTCCGTTATCAACTTG |
| underlined) | AAAAAGTGGCACCGAGTCGGTGCTTTTTT (SEQ ID |
| | NO: 5) |
| Leader sequence of CAR | MALPVTALLLPLALLLHAARP (SEQ ID NO: 6) |
| (CD8) | |
| Anti-binding domain of | EIVMTQSPATLSLSPGERATLSCRASQDISKYLNWYQQK |
| CAR (CD19 scFv) | PGQAPRLLIYHTSRLHSGIPARFSGSGSGTDYTLTISSLQP |
| | EDFAVYFCQQGNTLPYTFGQGTKLEIKGGGGSGGGGSG |
| | GGGSGGGGSQVQLQESGPGLVKPSETLSLTCTVSGVSL |
| | PDYGVSWIRQPPGKGLEWIGVIWGSETTYYQSSLKSRV |
| | TISKDNSKNQVSLKLSSVTAADTAVYYCAKHYYYGGS |
| | YAMDYWGQGTLVTVSS (SEQ ID NO: 7) |
| Transmembrane domain of | TTTPAPRPPTPAPTIASQPLSLRPEACRPAAGGAVHTRGL |
| CAR (CD8 TM) | DFACDIYIWAPLAGTCGVLLLSLVITLYC (SEQ ID NO: 8) |
| Intracellular costimulatory | KRGRKKLLYIFKQPFMRPVQTTQEEDGCSCRFPEEEEG |
| signaling domain of CAR | GCEL (SEQ ID NO: 9) |
| (41BB) | |
| Intracellular primary | RVKFSRSADAPAYQQGQNQLYNELNLGRREEYDVLDK |
| signaling domain of CAR | RRGRDPEMGGKPRRKNPQEGLYNELQKDKMAEAYSEI |
| (CD3) | GMKGERRRGKGHDGLYQGLSTATKDTYDALHMQALP |
| | PR (SEQ ID NO: 10) |
| T2A linker peptide | SGGGGEGRGSLLTCGDVEENPGPR (SEQ ID NO: 11) |
| (Optional) | |
| mCherry (optional) | MVSKGEEDNMAIIKEFMRFKVHMEGSVNGHEFEIEGEG |
| | EGRPYEGTQTAKLKVTKGGPLPFAWDILSPQFMYGSKA |
| | YVKHPADIPDYLKLSFPEGFKWERVMNFEDGGVVTVT |
| | QDSSLQDGEFIYKVKLRGTNFPSDGPVMQKKTMGWEA |
| | SSERMYPEDGALKGEIKQRLKLKDGGHYDAEVKTTYK |
| | AKKPVQLPGAYNVNIKLDITSHNEDYTIVEQYERAEGR |
| | HSTGGMDELYK (SEQ ID NO: 12) |

*gRNA sequences are shown as nucleotide sequences; CAR components and other protein components are shown as amino acid sequences

### CAR T Cells can be Genetically Modified to Reduce IFNγ Production

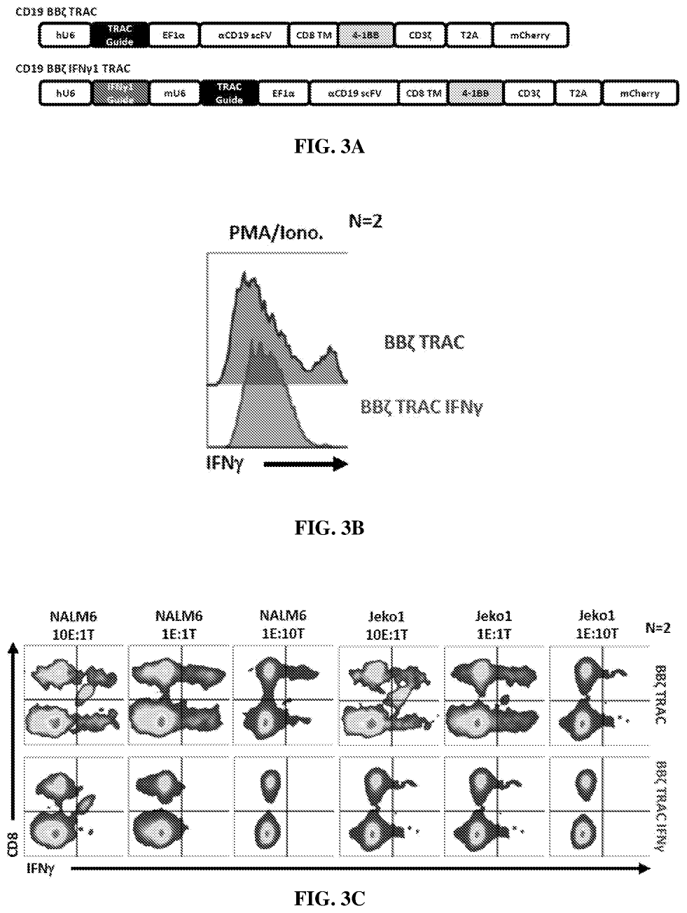

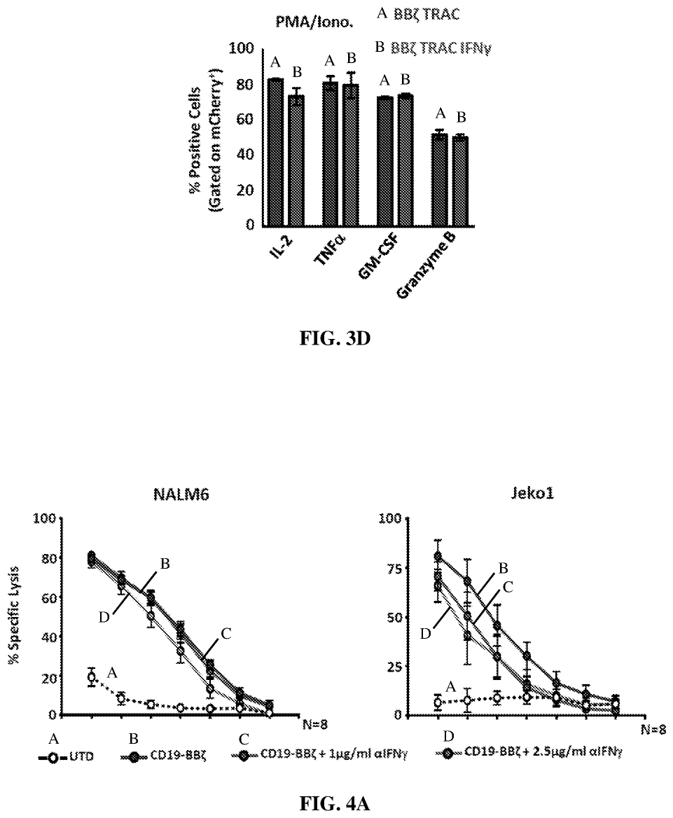

Lentiviral constructs were used to express CD19 CARs and to target the endogenous T cell receptor (TRAC) or IFNγ and TRAC (FIG. 3A). T cells isolated from healthy donors were stimulated with CD3 and CD28 beads for 24 hours before transducing with the lentiviral constructs shown in in FIG. 3A. On Day 5, the beads were removed and the cells were electroporated with Cas9 mRNA to initiate CRISPR-mediated disruption of the genes targeted by the guides (TRAC and/or IFNγ). On day 10, cells no longer expressing TRAC were isolated by column purification or flow-based sorting for CD3− cells. On day 12, cells were activated with PMA and Ionomycin for 6 hours or with NALM6 or Jeko1 cells at varying E:T ratios for 18 hours and stained for flow cytometry. Intracellular IFNγ expression was quantified in BBζ TRAC and BBζ TRAC IFNγ CAR T cells using flow cytometry. The data demonstrate that CRISPR-mediated disruption of IFNγ decreases its expression on CAR T cells (FIG. 3B). Cell surface CD8 and intracellular IFNγ expression by CAR T cells activated with NALM6 or Jeko1 cells at various E:T ratios were quantified using flow cytometry. The data demonstrate that CAR T cells transduced with the BBζ TRAC IFNγ construct do not express IFNγ across an array of activation conditions (FIG. 3C). Measuring cytokine expression in BBζ TRAC and BBζ TRAC IFNγ CAR T cells stimulated with PMA and Ionomycin by flow cytometry, reported as the percent of CAR T cells (mCherry+ cells) positive for the cytokine stain, showed that genetic depletion of IFNγ in CAR T cells did not affect their expression of IL-2, TNFα, GM-CSF or granzyme B in response to activation with PMA and Ionomycin (FIG. 3D). Combined, these results demonstrate that human T cells were able to be genetically modified to specifically reduce IFNγ production while simultaneously expressing a CAR, and that this genetic manipulation surprisingly did not affect the production of other cytokoines when CAR T cells were activated.

Pharmacologic or Genetic Depletion of IFNγ does not Reduce CAR T Cell Killing of Hematologic Cancer Cell Lines In Vitro

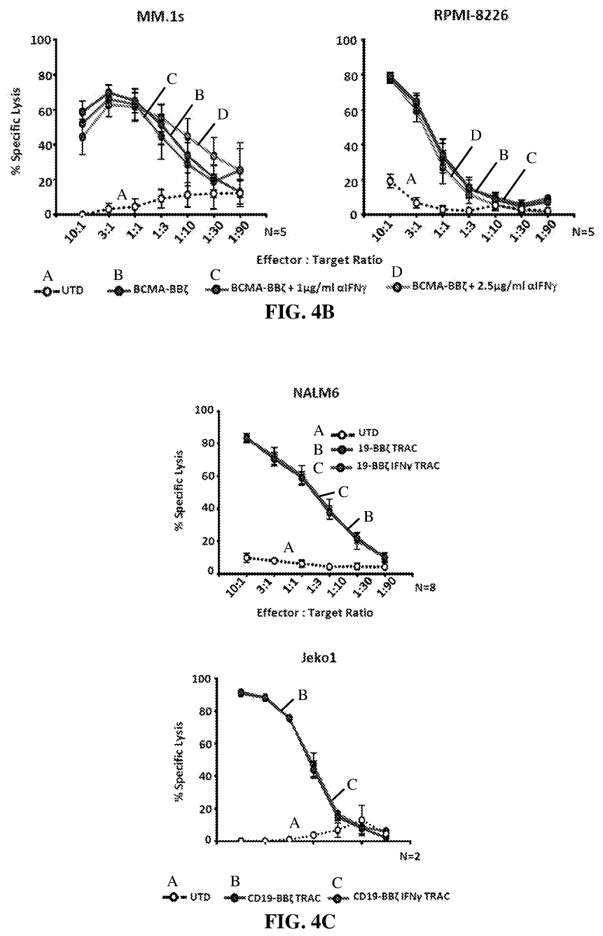

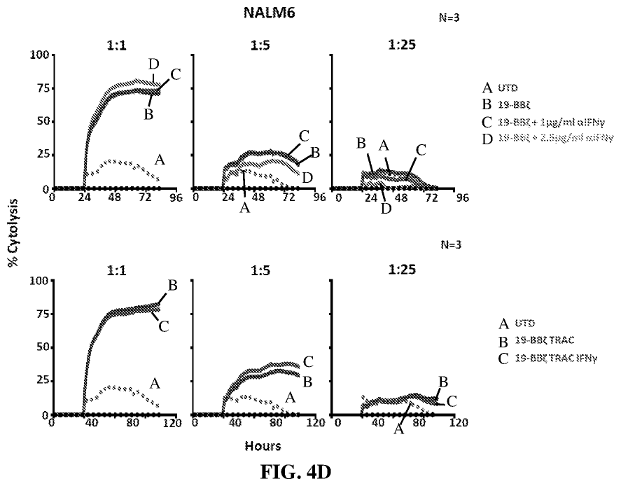

CD19-41BBζ or BCMA-41BBζ CAR T cells were produced as described above and expanded for 14 days. Following treatment with varying concentrations of anti-IFNγ, CD19-41BBζ CAR T cells were activated by mixing with CD19-expressing NALM6 leukemia or Jeko1 lymphoma cells at various E:T ratios overnight. Cell-specific lysis was measured by a luciferase-based killing assay, and demonstrated that treatment with anti-IFNγ did not affect CAR T cell killing of these liquid tumor cells in vitro (FIG. 4A). Similarly, BCMA-41BBζ CAR T cells treated with various concentrations of anti-IFNγ were activated by mixing with BCMA-expressing MM.1s or RPMI-8226 myeloma cells at various E:T ratios overnight and cell-specific lysis was measured. Again, IFNγ blockade did not affect CAR T cell killing of these hematological cancer cell lines (FIG. 4B). Genetic depletion of IFNγ also did not affect CAR T cell killing of hematological cancer cell lines. BBζ TRAC and BBζ TRAC IFNγ CAR T cells were produced as described above and activated by mixing with NALM6 or Jeko1 cells various E:T ratios overnight. Cell-specific lysis was measured by a luciferase-based killing assay, and demonstrated that genetic depletion of IFNγ expression by CD19-CAR T cells did not affect their cytolytic activity against NALM6 or Jeko1 cells (FIG. 4C). CD19-41BBζ (FIG. 4D, top) or BBζ TRAC and BBζ TRAC IFNγ (FIG. 4D, bottom) CAR T cells were prepared as described above. CD19-41BBζ cells were treated with two different doses of anti-IFNγ antibody, then mixed with NALM6 cells at varying E:T ratios and tracked by ACEA assay for 96 hours to measure percent cytolysis. Treatment with anti-IFNγ did not affect the ability of CD19-CAR T cells to kill NALM6 cells (FIG. 4D, top). Similarly, genetic depletion of IFNγ in CD19-CAR T cells did not affect their ability to kill NALM6 cells (FIG. 4D, bottom). Together, these data show that inhibition of IFNγ does not affect CAR T cell killing of liquid tumor cells in vitro.

Pharmacologic or Genetic Depletion of IFNγ does not Reduce CAR T Cell Killing In Vivo

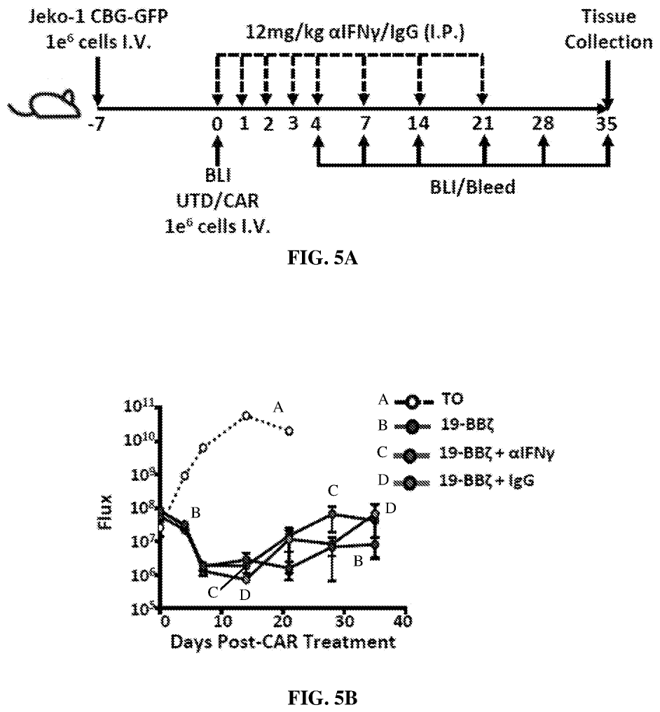

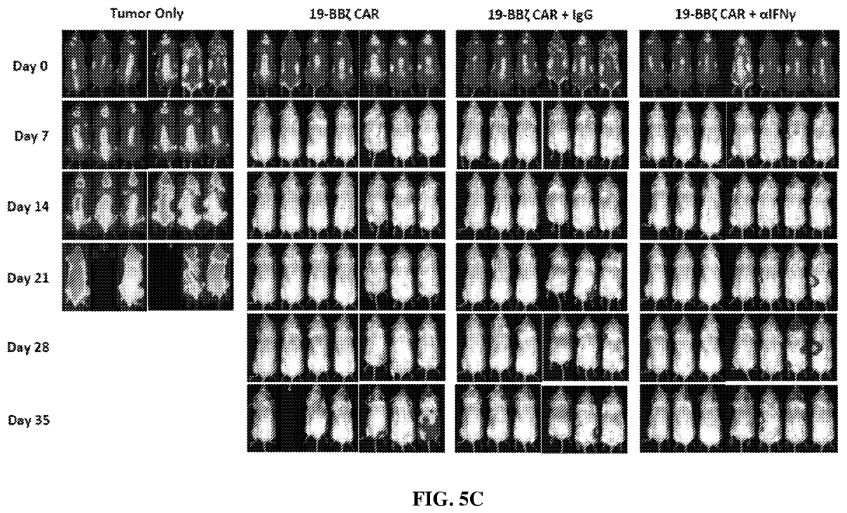

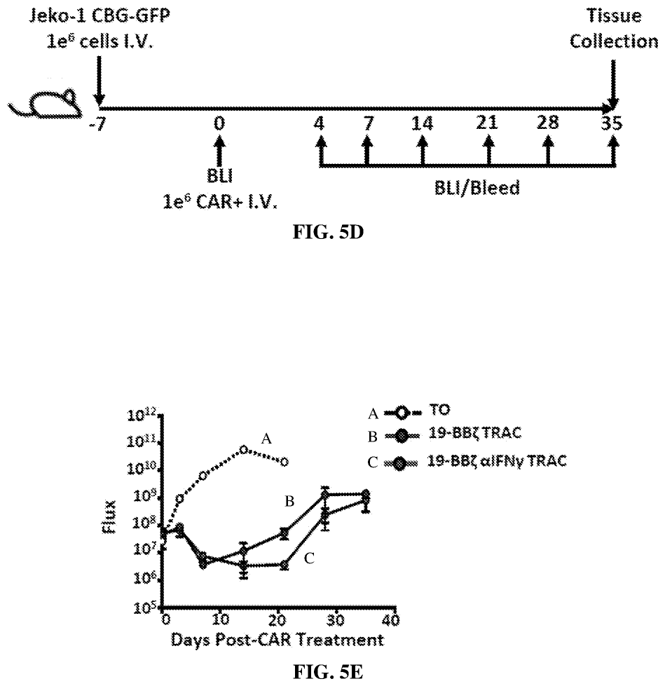

To test the effect of IFNγ depletion within CAR T cells on their killing activity, NOD-SCID gamma (NSG) mice were engrafted via intravenous (IV) injection with 1e6 Jeko-1 lymphoma cells expressing click beetle green luciferase and GFP reporters (Jeko-1 CBG-GFP cells). Seven days later, mice were administered 1e6 CAR T cells IV and tumor burden was measured by bioluminescence imaging. Bioluminescence was measured 4, 7, 14, 21, 28 and 35 days after administration of the CAR T cells. On these days, mice were also bled to evaluate the presence of CAR T cells. Mice were injected with untransduced (UTD) T cells or CAR T cells expressing CD19-41BBζ and either were not antibody-treated, or were treated with anti-IFNγ, or control IgG antibody on the specific days (FIG. 5A). Average bioluminescence demonstrated that antibody-mediated IFNγ blockade had no impact on CAR T cell anti-tumor efficacy, as mice treated with CD19-BBζ, CD19-BBζ+IgG, and CD19-BBζ+anti-IFNγ all showed substantially lower bioluminescence flux than mice treated with untransduced T cells, and the bioluminescence flux within these three groups was similar between these three treatment groups (FIGS. 5B-5C). Treatment of Jeko-1 tumor-bearing mice with CAR T cells genetically depleted of IFNγ demonstrated similar results. Mice were injected with UTD, BBζ TRAC, or BBζ TRAC IFNγ CAR T cells 7 days after being engrafted with Jeko-1 CBG-GFP cells (FIG. 5D). Average bioluminescence flux and bioluminescence images demonstrated that CRISPR-mediated IFNγ blockade did not negatively affect CAR T cell efficacy against Jeko-1 lymphoma cells (FIGS. 5E-5F).

CAR T Cells Lacking IFNγ Differentially Interact with Macrophages

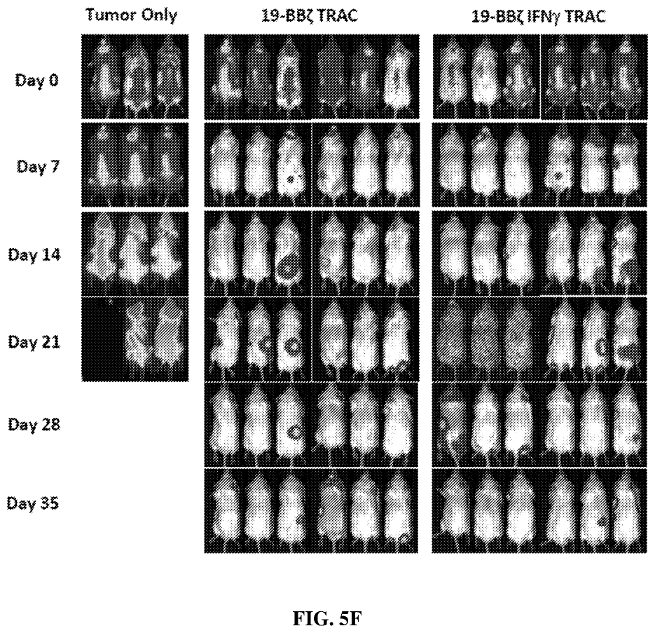

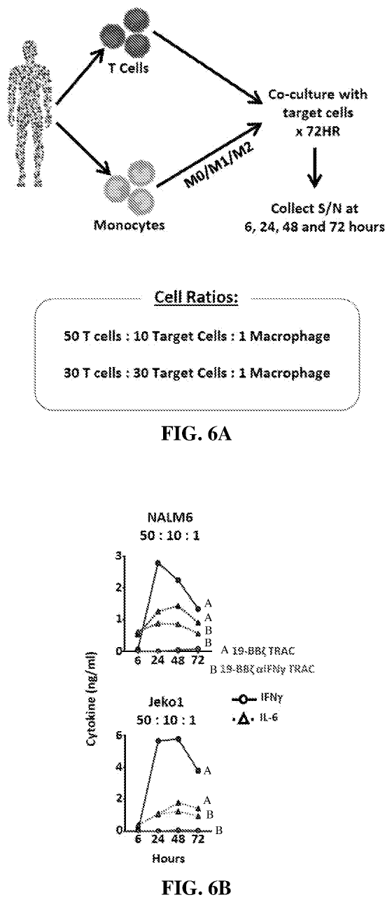

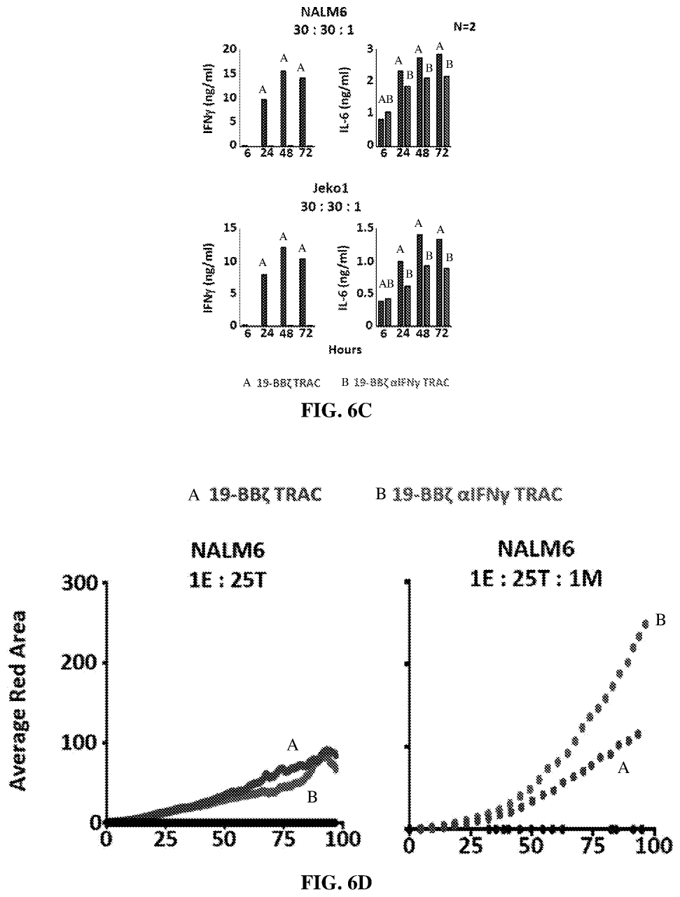

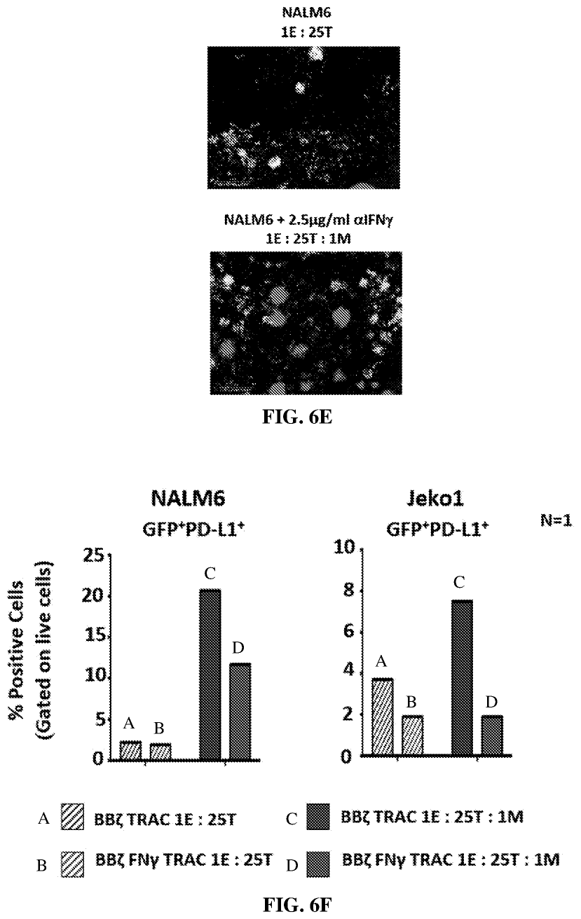

To test the effect of IFNγ blockade on the interaction between CAR T cells and macrophages, macrophages were first generated from the same healthy donor blood as CAR T cells. Monocytes were isolated and stimulated with M-CSF (to generate M0-phenotype macrophages), GM-CSF, IFNγ, and LPS (to generate M1-phenotype macrophages), or M-CSF, IL-4, and IL-13 (to generate M2-phenotype macrophages) for seven days. The stimulated monocytes were then rested for 5-7 days prior to being mixed with BBζ TRAC or BBζ TRAC IFNγ CAR T cells and NALM6 or Jeko1 cancer cells at various ratios. Cells were mixed and supernatant was collected 6, 24, 48 and 72 hours later (FIG. 6A). Measurement of IFNγ and IL-6 over time in supernatant from cell mixtures (at ratios of 50 T cells: 10 target cells: 1 macrophage or 30 T cells: 30 target cells: 1 macrophage) showed that genetic depletion of IFNγ in CAR T cells suppressed both IFNγ and IL-6 production by the cell co-cultures (FIGS. 6B-6C). To monitor the effect of macrophages on the expansion of CAR T cells over time, co-cultures were initiated at a ratio of 1 CAR T cell:25 tumor cells, with or without 1 macrophage. Fluorescence was measured over 96 hours on an Incucyte® live cell analysis system. CAR T cells were detected by mCherry expression and cancer cells by GFP. Fluorescence analysis showed that the presence of macrophages in the co-cultures enhanced the expansion of CAR T cells lacking IFNγ expression relative to IFNγ-wild-type CAR T cells (FIG. 6D). Representative images taken from NALM6 E:T and NALM6 E:T:M cultures at 96 hours show expansion of CAR T cells in cultures in with IFNγ blockade (FIG. 6E). IFNγ expression by CAR T cells also affected PD-L1 expression on macrophages, which inhibits T cell activity. CAR T cells were mixed at low T cell:cancer cell or T cell:cancer cell:macrophage ratios with NALM6 or Jeko1 cells for 96 hours, and PD-L1 expression on the cancer cells was measured by flow cytometry, which showed decreased PD-L1 expression on cancer cells in co-cultures with macrophages and CAR T cells lacking IFNγ expression (FIG. 6F).

### Genetic Disruption of IFNγ Expression in CAR T Cells Reduces Cytokine Chemokine Production and Adhesion Molecule Expression in the Presence of Macrophages

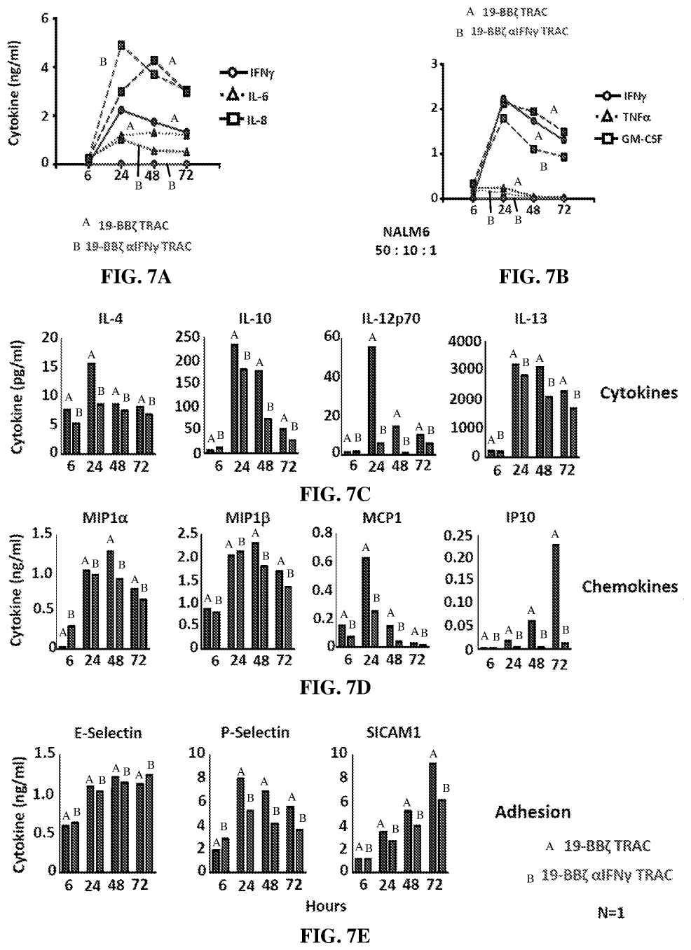

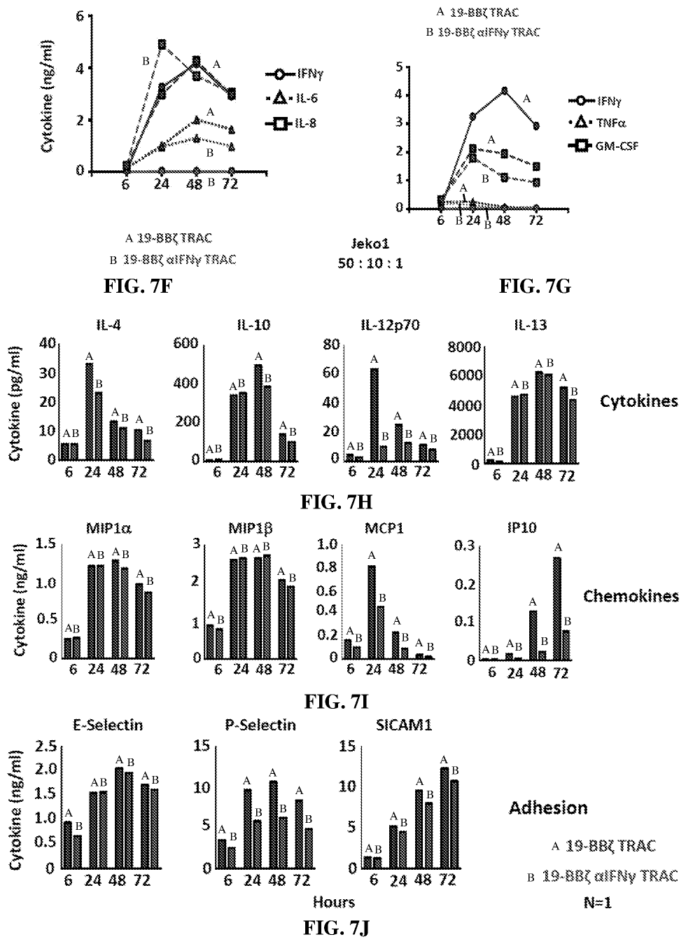

In the absence of IFNγ production, macrophage response to CAR T and tumor cells is subdued, as demonstrated by reduced expression of IL-6, MCP-1 and IP-10 in CAR T cell, cancer cell, macrophage co-cultures. NALM6 or Jeko-1 cells were mixed with macrophages and BBζ TRAC or BBζ TRAC IFNγ CAR T cells at a ratio of 50 T cells:10 cancer cells:1 macrophage, and cytokines, chemokines, and adhesion molecules were measured in co-culture supernatant using a Luminex kit. In the case of NALM6 cells, at least IFNγ, IL-6, GM-CSF, IL-4, IL-10, IL-12p70, IL-13, MCP1, IP-10, P-selectin, and SICAM1 were decreased in supernatants of co-cultures containing CAR T cells deficient in IFNγ relative to those with IFNγ-wild-type CAR T cells (FIGS. 7A-7E). When Jeko-1 cells were used in the co-cultures, at least IFNγ, IL-6, IL-4, IL-10, IP-12p70, MCP1, IP-10, P-selectin, and SICAM1 were decreased in supernatants of co-cultures containing CAR T cells deficient in IFNγ relative to those with IFNγ-wild-type CAR T cells (FIGS. 7F-7J). This diminished macrophage response appears to yield less forward-feedback in T cells as decreased cytokines. Collectively, these data suggest that the production of IFNγ from activated CAR T cells is important for the full activation and function of macrophages and suggests that limiting its expression could reduce macrophage-mediated toxicities.

### IFNγ Blockade in CAR T Cells May Reduce the Killing of Glioblastoma Cells

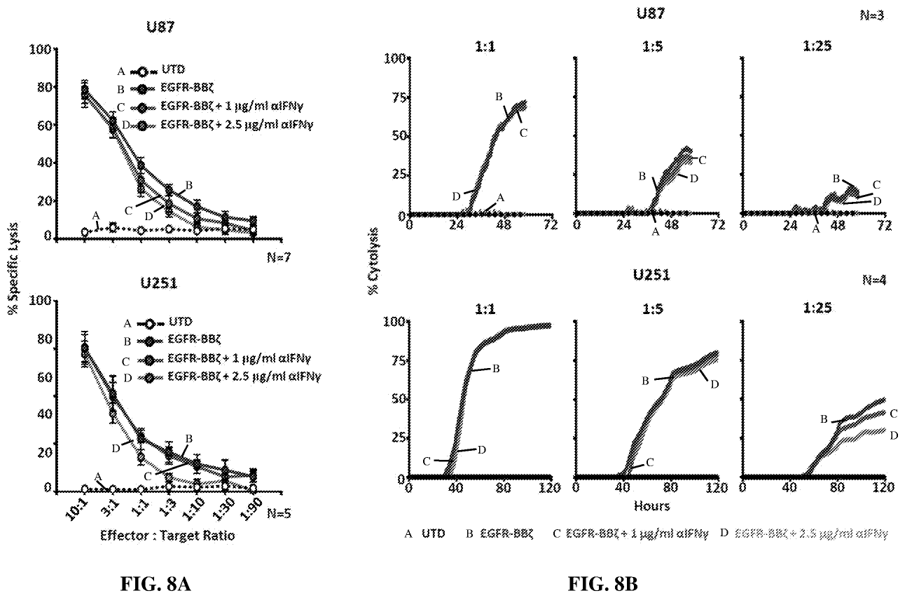

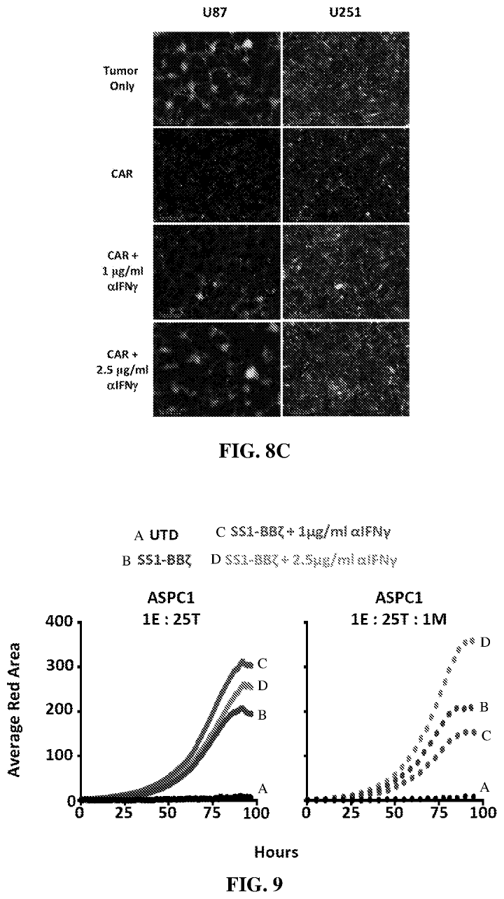

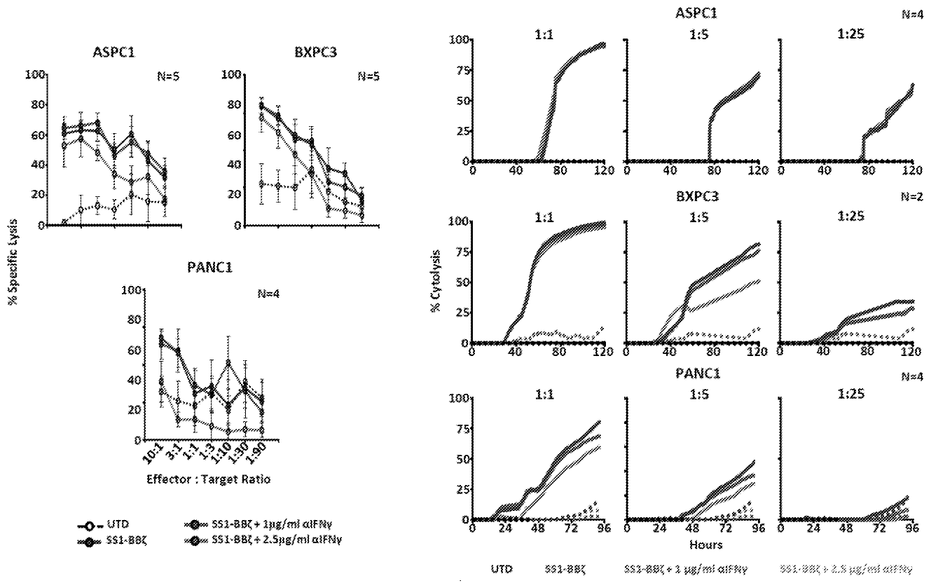

CAR T cells specific to the glioblastoma cell antigen EGFR were engineered and expanded for 14 days. They were then treated with varying doses of anti-IFNγ antibody and mixed with the glioblastoma cell lines U87 and U251 at varying effector:cancer cell ratios. Following an overnight incubation, cell specific killing was measured by a luciferase-based killing assay. Results demonstrated that CAR T cell killing of glioblastoma cells may be slightly reduced in the presence of anti-IFNγ antibody but still effective (FIG. 8A). Confirming these results in a second assay, following a 120-hour incubation, percent cytolysis was measured using an ACEA cytolysis assay. For this incubation, anti-IFNγ antibody was added every 24 hours. Results again demonstrated that CAR T cell killing of glioblastoma cells, especially U251 cells, may be slightly reduced in the presence of IFNγ blockade but still effective (FIG. 8B). Fluorescence images captured via an Incucyte® live cell analysis system following a 96 hour incubation with anti-IFNγ antibody (which was replenished every 24 hours), demonstrated that the relative cell density of tumor cells (identified by GFP expression) was higher in cancer cell/CAR T cell co-cultures treated with anti-IFNγ (FIG. 8C), suggesting that when glioblastoma cells (representing a solid tumor) are targeted with CAR T cells in the presence of IFNγ blockade, cell-specific killing of the tumor cells is slightly reduced but still effective.

Macrophages Facilitate Increased Expansion of CAR T Cells when IFNγ is Inhibited

Macrophages and CAR T cells were generated from the same healthy donor as described above. CAR T cells specific to the prostate cancer antigen SS1 (SS1-BBζ) or untransduced T cells (UTD) were co-cultured at ratios of 1 T cell:25 cancer cells or 1 T cell:25 cancer cells:1 macrophage and incubated with or without anti-IFNγ antibody for 96 hours. Relative CAR T cell proliferation was measured on an Incucyte® live cell analysis system by quantifying mCherry expression. CAR T cell proliferation results suggest that when IFNγ is inhibited, CAR T cell expansion is increased in response to activation by target cell interaction. These findings are similar to those seen in liquid tumors, suggesting that the reduction of IFNγ could be triggering increased CAR T proliferation, possibly by reducing PD-L1 expression on tumor cells.

### IFNγ Blockade in CAR T Cells May Reduce the Killing of Pancreatic Cancer Cells

CAR T cells specific to the pancreatic cancer cell antigen SS1 were engineered and expanded for 14 days. They were then treated with varying doses of anti-IFNγ antibody and mixed with the pancreatic cancer cell lines ASPC1, BXPC3 or PANC1 at varying T cell:cancer cell ratios. Following an overnight incubation, cell specific killing was measured by a luciferase-based killing assay. Results demonstrated that, similar to glioblastoma cells, pancreatic cancer cells were slightly less likely to be killed by CAR T cells when IFNγ was inhibited, as co-cultures treated with higher concentrations of anti-IFNγ antibody showed decreased specific lysis compared to co-cultures with less or no anti-IFNγ antibody (FIG. 10A). Similar measurements conducted over a 96-hour incubation, demonstrated that percent cytolysis, as measured using an ACEA assay, was in some cases reduced by the introduction of anti-IFNγ into CAR T/pancreatic cancer cell co-cultures, in which anti-IFNγ antibody was added every 24 hours (FIG. 10B).

### Materials and Methods

### CAR T Cell Production

T cells were isolated from healthy donor blood, re-suspended in R10 media (RPMI 1640+10% FBS+Pen/Strep), supplemented with 100 IU/ml IL-2 and stimulated with beads coated with anti-CD3 and anti-CD28 antibodies at a 1 T cell: 3 bead ratio. 24 hours post-activation, T cells were transduced with a lentiviral vector encoding the CAR+/−TRAC/IFNγ guides. On day 5, cells were de-beaded. In experiments where IFNγ was pharmacologically inhibited, the cells were treated with anti-IFNγ antibody for one hour on day 9 or 10 prior to performing functional assays.

### CAR T Cell Activation In Vitro

CAR T cells were activated in vitro either by treating them with PMA and Ionomycin or by co-incubating them with target cells. When co-incubating cells, varying ratios of effector (CAR T) cells to target (cancer) cells (E:T) were used to show a dose-dependent effect. CAR T cell activation was measured by cytokine production, cell specific lysis of the target cells, or expression of activation markers on the cell surface. Cytokine production was measured using an ELISA for the specific cytokine or by using a Luminex panel. IFNγ, GM-CSF, Granzyme B, and TNFα were measured using Human DuoSet kits from R&D. Other cytokines were measured using Th1/Th2 Luminex kits. Cell-specific lysis of target cells was measured by a change in detection of luciferase or GFP in cell cultures. Target (cancer) cells express both luciferase and GFP, while CAR T cells do not, so a decrease in either luciferase or GFP when target cells are mixed with CAR T cells indicates target cell-specific lysis. Luciferase expression was measured by a luciferase assay. GFP was measured on an Incucyte® live cell analysis system, which detects relative amounts of GFP and can simultaneously measure relative numbers of CAR T cells by detecting mCherry. Cell surface expression of activation markers (CD69 and CD107a) was measured by flow cytometry on an LSR Fortessa flow cytometer.

### Knocking Out IFNγ in CAR T Cells

IFNγ was disrupted in CAR T cells using a CRISPR/Cas9 system. Lentiviral constructs were designed to simultaneously express a CAR as well as small guide RNAs (sgRNAs) for IFNγ and/or TRAC. TRAC guides were used to target the endogenous T cell receptor. These cells were produced as described above, but after the stimulation beads were removed, the cells were electroporated with 10 g Cas9 mRNA. Cells with decreased expression of target genes were then identified by the absence of CD3 expression. These cells were isolated by CD3 column purification or flow-based cell sorting on day 8. CD3− cells were then used for activation assays.

### CAR T Cell Killing In Vivo

6-8 week old NOD-SCID gamma (NSG) mice were intravenously injected with 1e6 Jeko-1 or NALM6 CBG-GFP+ cells. Seven days later, mice were left untreated (tumor only; TO) or were injected with 1e6 CAR T cells IV and tumor burden was measured by bioluminescence imaging. CAR T cells were grown as previously described. Mice receiving the anti-IFNγ blocking antibody or control IgG antibody (both given at 12 mg/kg) were IP injected with the appropriate solutions 1 hour prior to CAR-T injection. Antibodies were administered IP every 24 hours for the first 5 days and then maintained with 1 injection/week for the remainder of the experiment. Bioluminescence was measured 4, 7, 14, 21, 28 and 35 days later. On these days, mice were also bled to look for 1) cytokine expression by ELISA/Luminex or 2) CAR-T persistence by flow cytometry.

### Macrophage Production

Macrophages were matured from human monocytes isolated from human peripheral blood. Macrophages were derived from the same healthy donor blood as CAR T cells by isolating monocytes and stimulating them with M-CSF (to promote an M0 phenotype), GM-CSF, IFNγ, and LPS (to promote an M1 phenotype) or M-CSF, IL-4, and IL-13 (to promote an M2 phenotype) for seven days. The monocytes were then rested for 5-7 days prior to mixing them with CAR T cells and cancer cells.

### CAR-T, Macrophage and Tumor Co-Cultures

Macrophages were mixed with CAR T cells and target (cancer) cells at ratios of 50 T cells: 10 target cells: 1 macrophage or 30 T cells: 30 target cells: 1 macrophage. Cells were combined and supernatant was collected at 6, 24, 48 and 72 hours into the assay to assess cytokines. Cytokine production was measured using Human DuoSet ELISA kits or the Human Inflammatory Panel 20-plex Luminex kit. CAR T cells were also mixed with cancer cells with and without macrophages to measure T cell proliferation, cell specific lysis and cancer cell PD-L1 expression. T cell proliferation and target cell killing were measured over the course of the 96 hours on an Incucyte® live cell imaging system by detection of mCherry and GFP expression, respectively. PD-L1 was measured by flow cytometry, after gating on live cells.

### Example 2 Neutralizing IFNγ Production by CAR T Cells

CAR T cells are an effective treatment option for some cancer patients with liquid tumors (i.e. leukemia, myeloma, or lymphoma), however they can have toxic side effects such as cytokine release syndrome. Normal cytotoxic T cells use ligand induced death, the release of perforin and granzyme, and IFNγ production to kill their target cells. However, it is unknown whether CAR T cells use these same mechanisms to kill cancer cells. It is also known that IFNγ production can lead to toxicity in patients by causing other cells of the immune system, macrophages, to over produce cytokines, which can lead to toxic inflammation (also known as cytokine release syndrome). To determine if IFNγ is required for CAR T cell killing and if reducing IFNγ production by CAR T cells can reduce cytokine production by macrophages, IFNγ production was knocked out in CAR T cells. If IFNγ is not required for CAR T cell killing, preventing CAR T cells from producing IFNγ could make them less toxic by preventing cytokine release syndrome.

### IFNγ can be Pharmaceutically Blocked in CAR T Cells

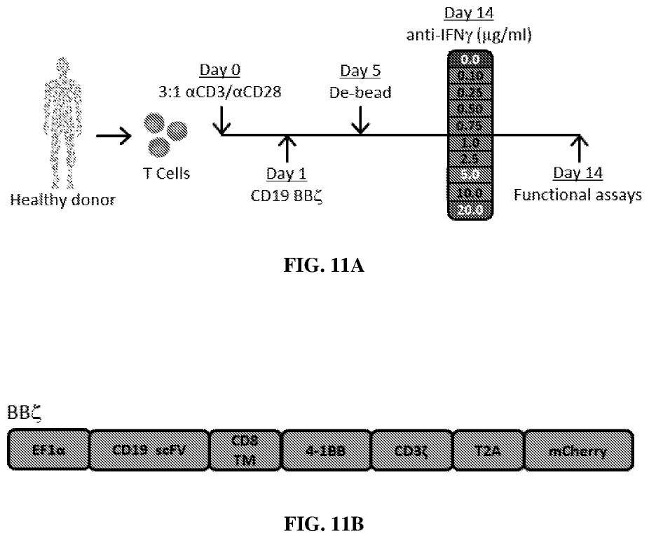

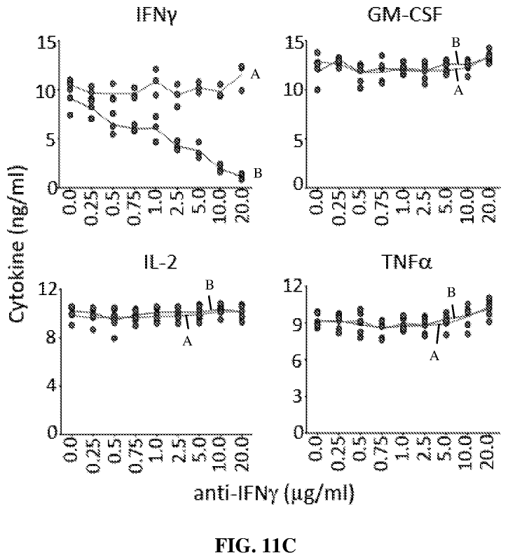

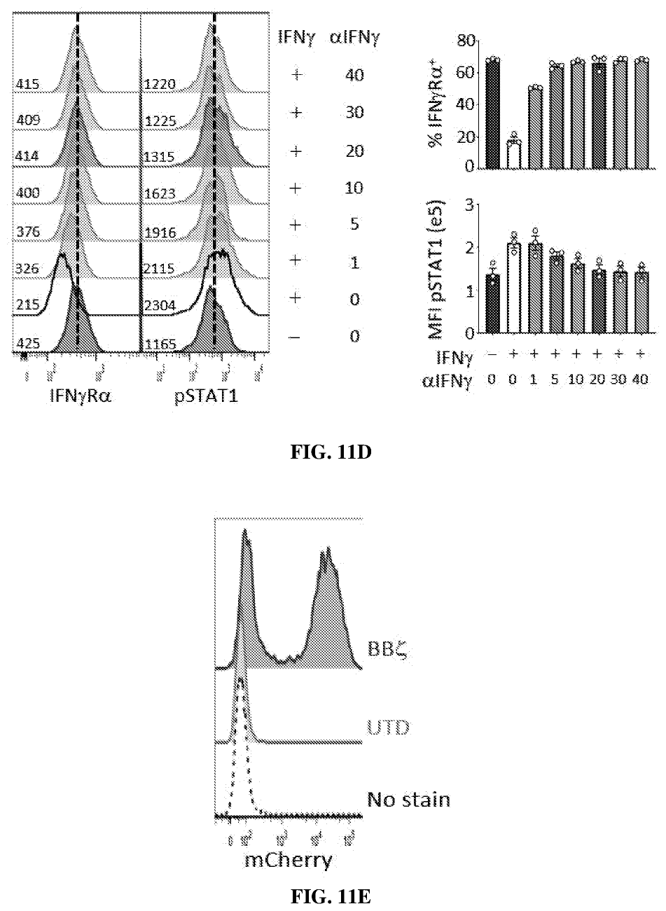

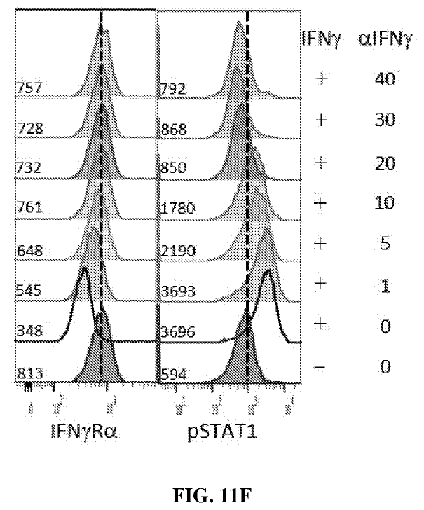

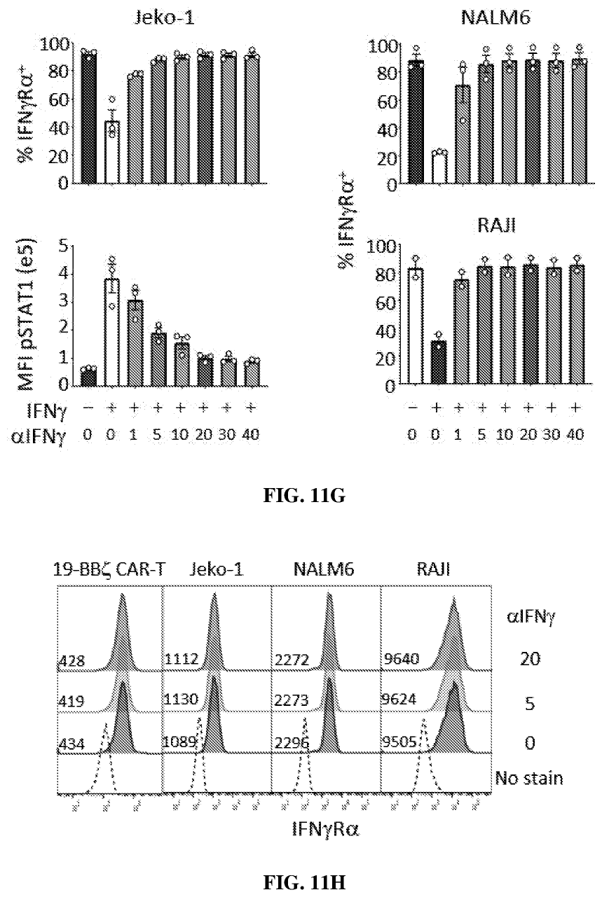

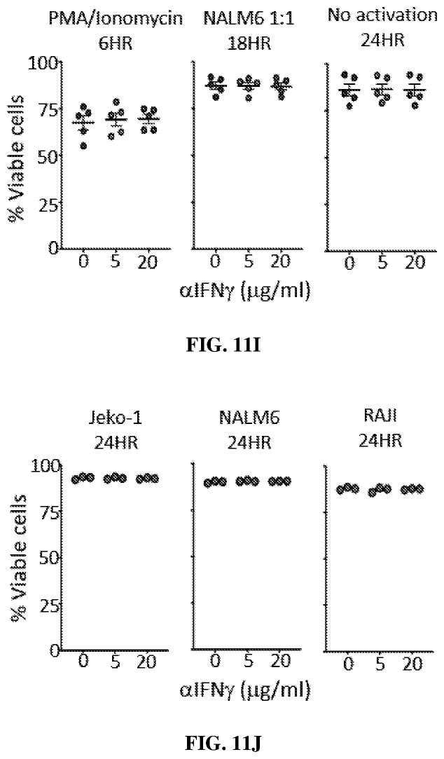

T cells isolated from healthy donors were stimulated with beads coated in CD3 and CD28 antibodies for 24 hours before transducing with a lentiviral vector to express a CD19-41BBζ CAR (FIG. 11A-11B). On Day 5, the stimulation beads were removed. On Day 9 or 10, the cells were treated with the indicated doses of anti-IFNγ antibody for one hour. Cells were incubated with varying doses of anti-IFNγ or isotype control (0.25-20 ug/ml) for 1 hr at 37 C. After 1 hour, cells were activated with PMA/Ionomycin ×6 hours at 37 C. Cells were centrifuged, supernatant collected and ELISAs were performed using the Human DuoSet Kits from R&D (N=3) (FIG. 11C). CAR cells were expanded and transduced as shown in FIG. 11A, given varying doses of anti-IFNγ for 1 hr at 37 C and then treated with 10 ng/ml recombinant human IFNγ×20 minutes at 37 C. IFNγRa expression and pSTAT1 signaling were assessed by flow cytometry (N=3) (FIG. 11D). CAR-T were created using the protocol shown in FIG. 11A and transduction efficiency (mCherry) was checked by flow cytometry (FIG. 11E). Cancer cell lines were given varying doses of anti-IFNγ for 1 hr at 37 C and then treated with 10 ng/ml recombinant human IFNγ×20 minutes at 37 C. IFNγRa expression and pSTAT1 signaling were assessed by flow cytometry (N=3) (FIG. 11F). Baseline IFNγRa expression on CAR-T and cancer cell lines was observed by flow cytometry (representative; N=3) (FIG. 11G-11H). CAR-T viability following incubation with anti-IFNγ blocking antibody was examined at 6 hours post PMA/Ionomycin, 18 hours post-Nalm6 and 24 hours on resting T cells (N=5) (FIG. 11I). Cancer cells lines were incubated in IFNγ blocking antibody for 24 hours prior to assessing cell viability by flow cytometry (N=3 experiments) (FIG. 11J).

Results in FIG. 11A-11J demonstrate that IFNγ can be blocked using an anti-IFNγ antibody or genetic targeting. Loss of IFNγ production by CAR T cells does not affect their overall functionality, as evidenced by their ability to continuing production other cytokines when activated. Furthermore, loss of IFNγ did not affect the viability or expression of IFNγRa on CAR-T. Reduced pSTAT1 signaling in CAR-T treated with anti-IFNγ blocking antibody confirms that IFNγ is being specifically targeted.

### IFNγ can be Genetically Targeted in CAR T Cells

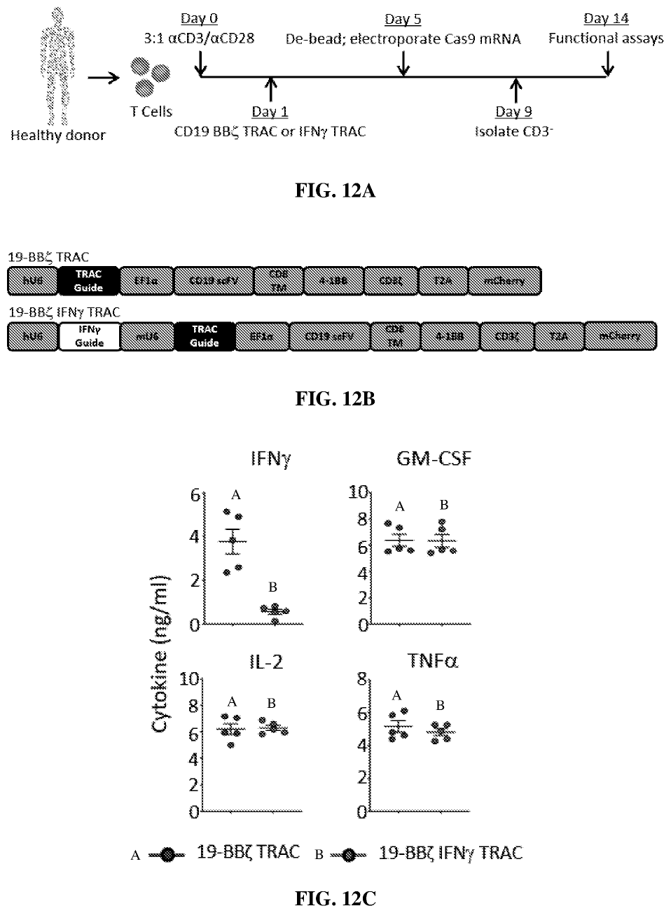

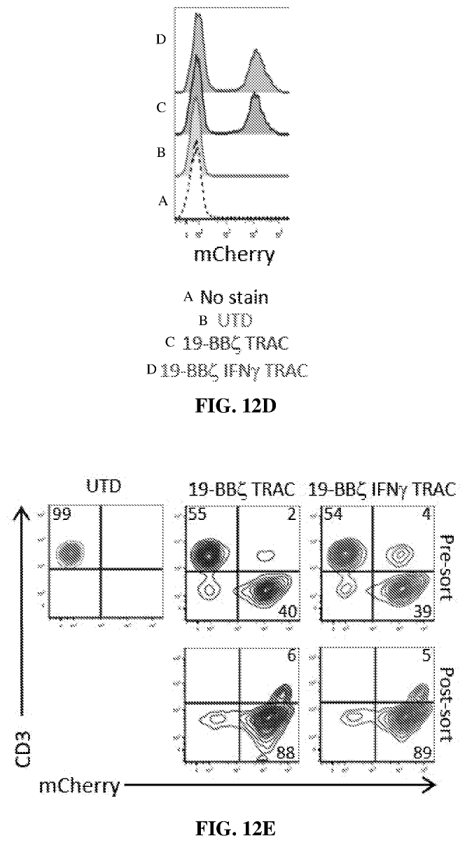

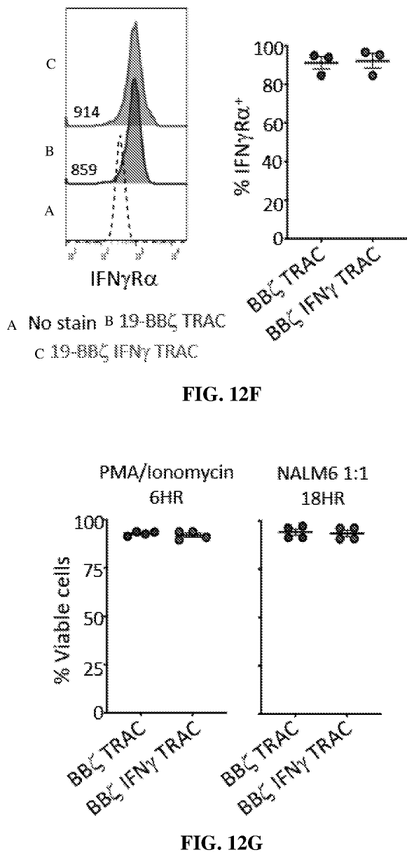

Human T cells were genetically modified to specifically reduce IFNγ production while simultaneously expressing the CAR. T cells isolated from healthy donors were stimulated with CD3 and CD28 beads for 24 hours before transducing with the lentiviral vectors (FIG. 12A-12B). On Day 5, the beads were removed and the cells were electroporated with Cas9 mRNA to initiate CRISPR-mediated deletion of the genes targeted by the guides (TRAC and/or IFNγ). On day 10, cells with successful deletion of TRAC were isolated by column purification of flow-based sorting for CD3− cells. Vector design for knockout CAR-T constructs (KO) with guide RNA to TRAC or TRAC and IFNγ is shown in FIG. 12B. KO CAR-T were activated with PMA/Ionomycin for 6 hours and IFNγ, CM-CSF, IL-2 and TNF-alpha cytokines were assessed by ELISA (N=3) (FIG. 12C). CAR-T were created using the protocol discussed in FIG. 12A and transduction efficiency was determined by flow cytometry (FIG. 12D). KO CAR-T were assessed for CD3 expression pre- and post-CD3 isolation by flow cytometry (representative of N=5) (FIG. 12E). Baseline levels of IFNγRa on KO CAR-T was assessed by flow cytometry (FIG. 12F). KO CAR-T were activated for 6 hours (PMA/Ionomycin) or 18 hours (Nalm6) before checking viability by flow cytometry (N=4) (FIG. 12G).

Results in FIG. 12A-12G demonstrate that IFNγ can be genetically targeted using lentiviral CAR constructs, which can be identified by the loss of CD3 expression (due to TRAC targeting). Genetic deletion of IFNγ in CAR T cells but does not affect their overall functionality, as evidenced by their ability to continuing production other cytokines when activated. Furthermore, loss of IFNγ did not affect the viability or expression of IFNγRa on CAR-T.

Pharmacologic or Genetic Depletion of IFNγ does not Reduce CAR T Cell Killing of Hematologic Cancer Cell Lines In Vitro

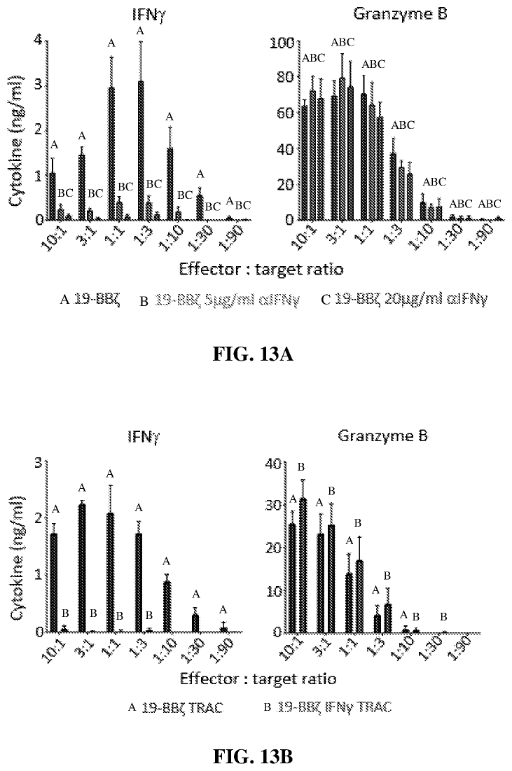

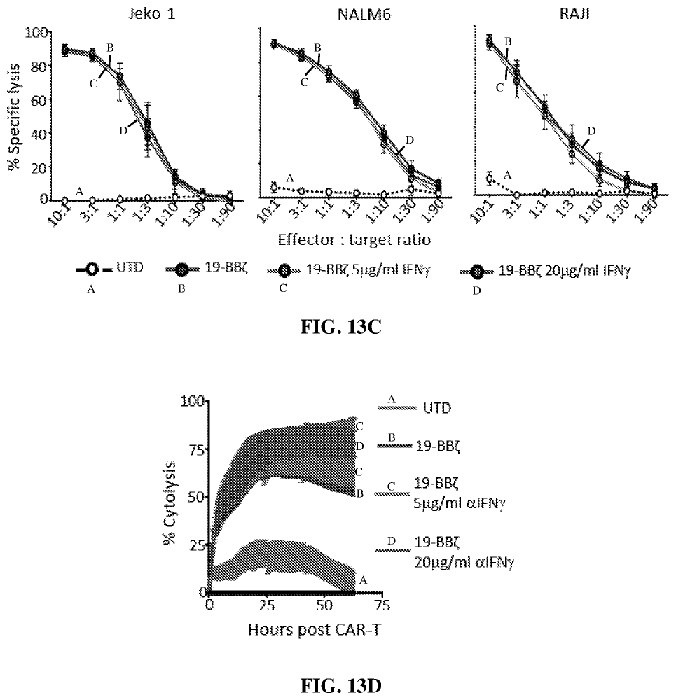

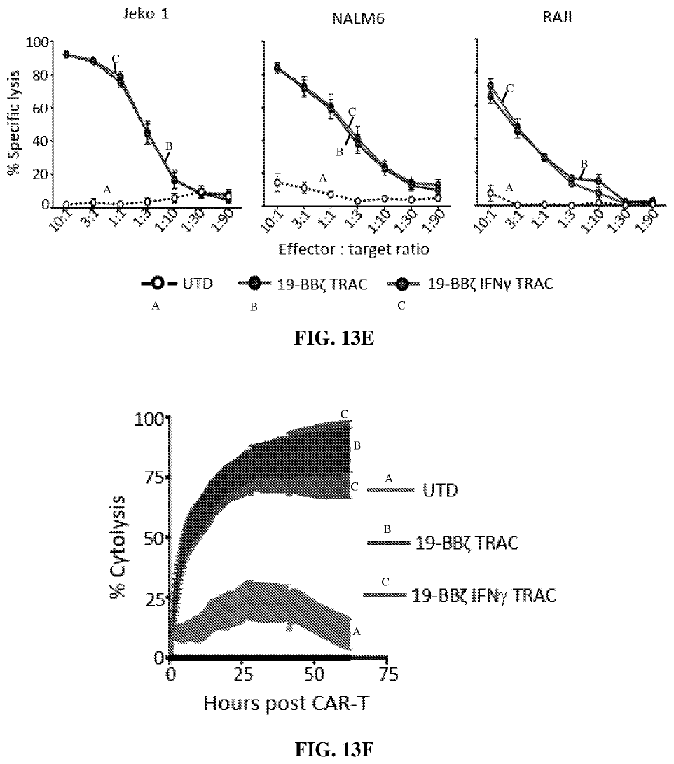

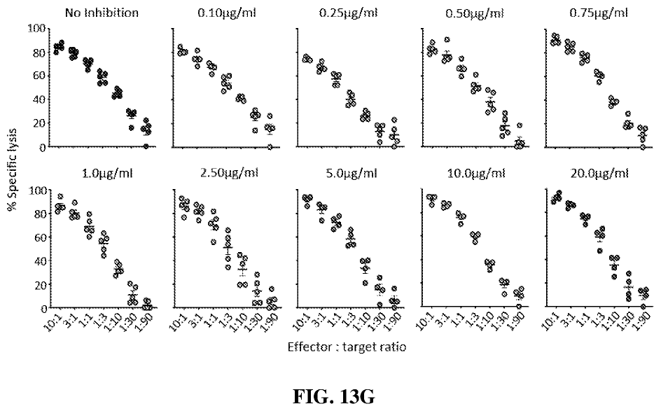

CD19-BBζ CAR T cells were produced as described and expanded for 14 days. Following treatment with varying concentrations of anti-IFNγ antibody, CAR T cells were activated with CD19-expressing NALM6 leukemia at the indicated E:T ratios overnight. Supernatant was collected and assessed for cytokine production by ELISA (N=5) (FIG. 13A). KO CAR T cells were produced as described and expanded for 14 days. CAR T cells were activated with CD19-expressing NALM6 leukemia at the indicated E:T ratios overnight. Supernatant was collected and assessed for cytokine production by ELISA (N=5) (FIG. 13B). Cell-specific lysis by CD19-BBζ CAR-T (described above) was measured by a luciferase-based killing assay (FIG. 13C) or ACEA (FIG. 13D) (N=5). Cell-specific lysis by KO CAR-T described in B was measured by a luciferase-based killing assay (FIG. 13E) or ACEA (FIG. 13F) (N=5). Long-term killing of KO CAR-T was assessed by Incucyte at various tumor burdens: low (10E:1T), moderate (1E:1T) and high (1E:10T) (N=5) (FIG. 13G). CD19-BBζ CAR T cells were produced as described and expanded for 14 days. Following treatment with varying concentrations of anti-IFNγ antibody, CAR T cells were activated with CD19-expressing NALM6 leukemia at the indicated E:T ratios overnight. Cell-specific lysis was measured by a luciferase-based killing assay (N=5) (FIG. 13G).

Results in FIG. 13A-13G demonstrate that inhibition of IFNγ does not affect CAR T cell degranulation or killing of liquid tumor cells in vitro.

Pharmacologic or Genetic Depletion of IFNγ does not Reduce CAR T Cell Killing of Leukemia In Vivo

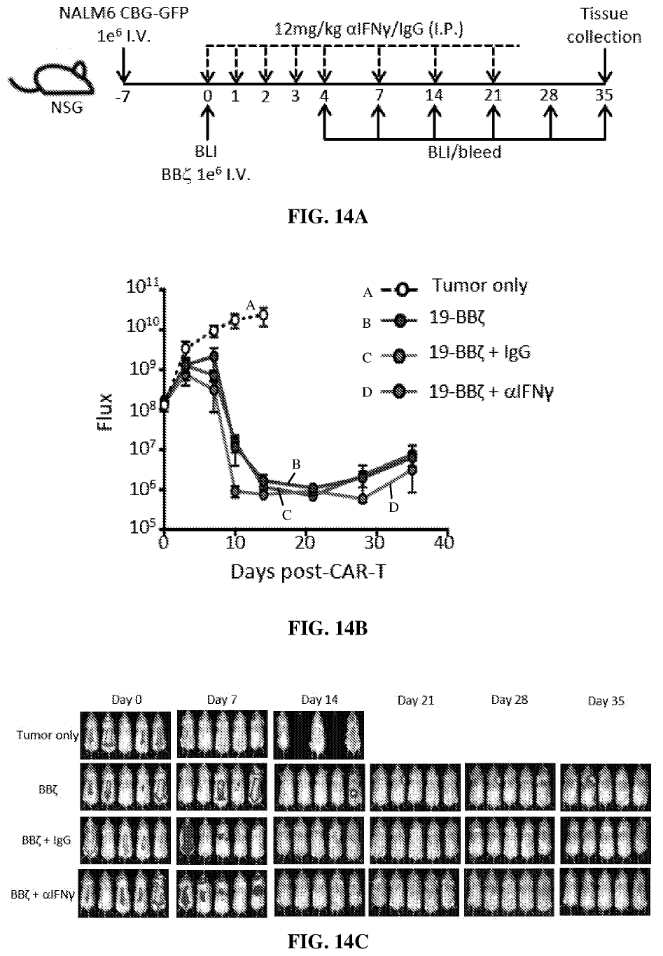

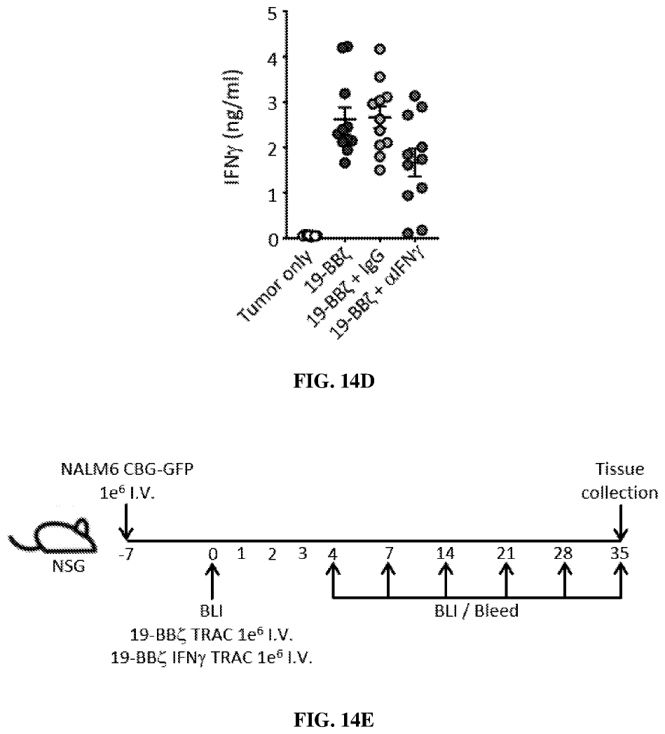

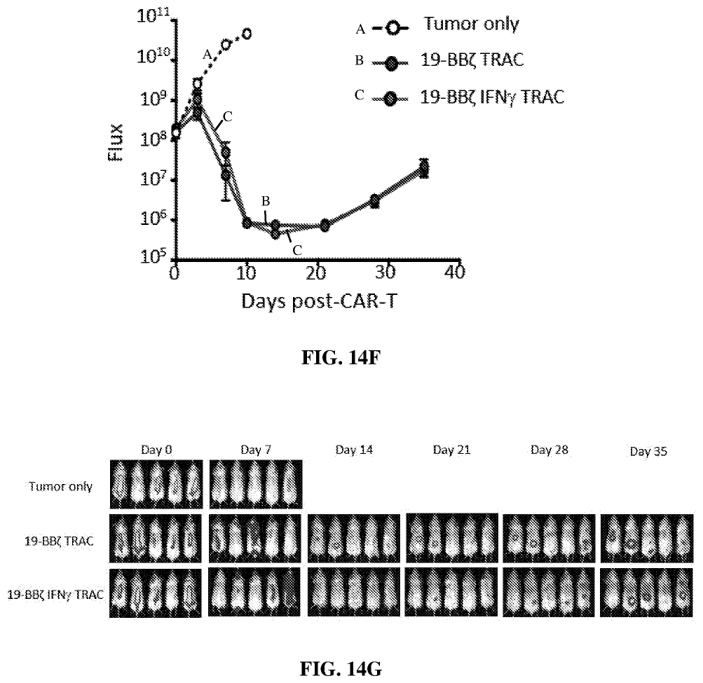

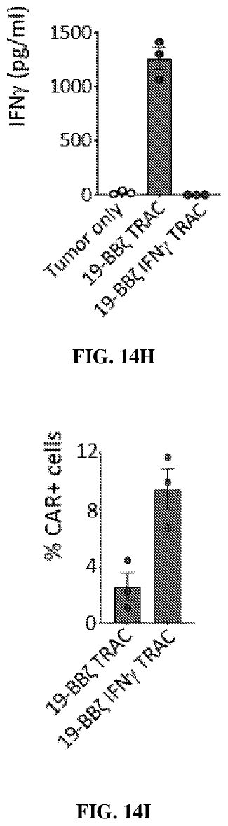

NSG mice were injected with 1e6 Nalm6 CBG-GFP cells IV. Seven days later, mice were injected with 1e6 CAR T cells IV and tumor burden was measured by bioluminescence. Bioluminescence was measured 4, 7, 14, 21, 28 and 35 days later. On these days, mice were also bled to look for the presence of CAR T cells. Mice were injected with CD19-41BBζ CAR T cells and either not treated or treated with anti-IFNγ or control IgG antibody on the days indicated (N=3-5 mice/group; 4 donors) (FIG. 14A). Average bioluminescence flux for each group of mice over time was measured and is shown in FIG. 14B. Bioluminescent images of the mice at each time point are shown in FIG. 14C. Mice were bled 3 days post-CAR-T and serum was tested for IFNγ expression by ELISA (N=3-5 mice/group; 4 donors) (FIG. 14D). Mice were injected with BBζ TRAC or BBζ TRAC IFNγ CAR T cells one week after Nalm6 injection (FIG. 14E). Average bioluminescence flux for each group of mice over time was measured and is shown in FIG. 14F. Bioluminescent images of the mice at each time point are shown in FIG. 14G. Mice were bled 3 days post-CAR-T, serum was collected and IFNγ was assessed by ELISA (FIG. 14H). Mice were bled 14 days post-CAR-T injection and CAR-T persistence was determined by flow cytometry (N=3-5 mice/group; 4 donors) (FIG. 14I).

Results in FIG. 14A-14H demonstrate that inhibition of IFNγ does not affect CAR T cell killing of leukemia cells in mice, but does effectively reduce the levels of IFNγ in the serum as seen by ELISA. CAR-T with a genetic loss of IFNγ appear to have greater persistence in the blood.

Pharmacologic or Genetic Depletion of IFNγ does not Reduce CAR T Cell Killing of Lymphoma In Vivo

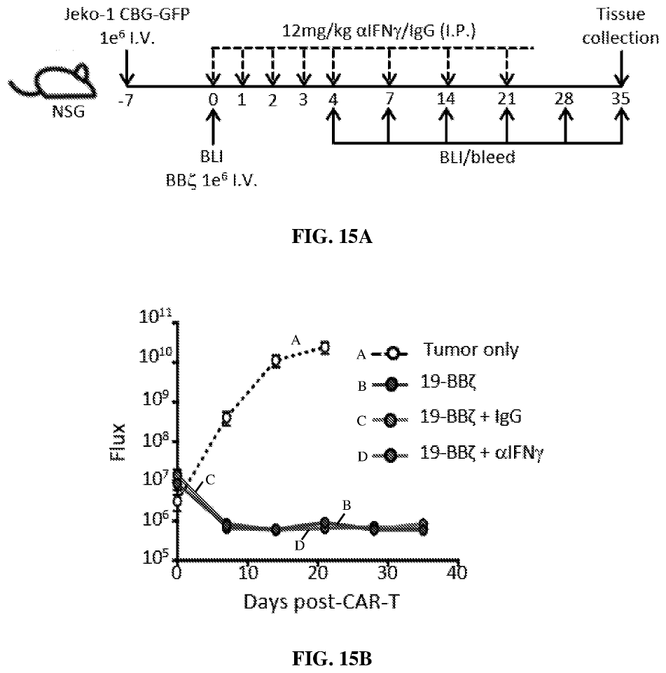

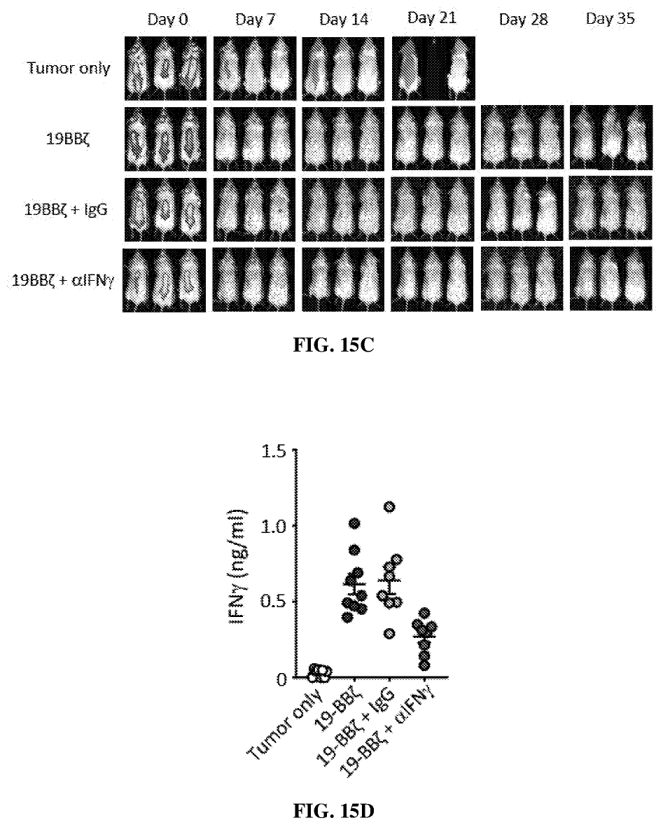

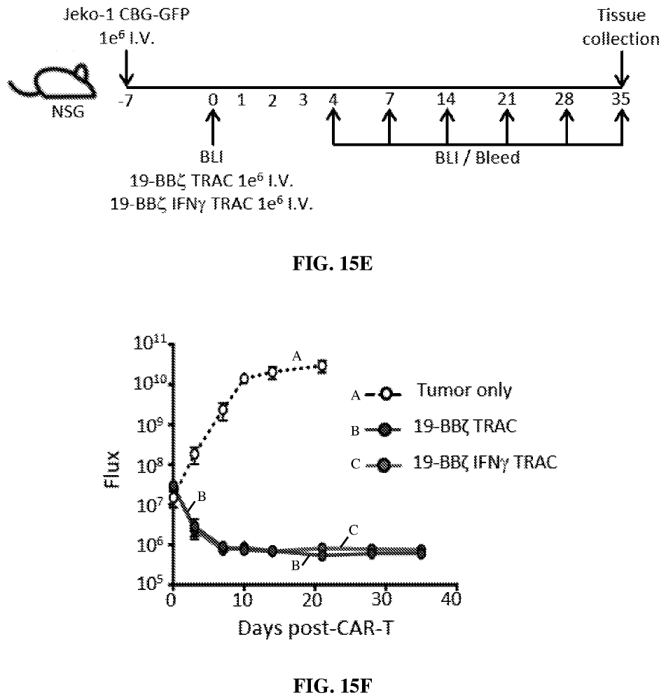

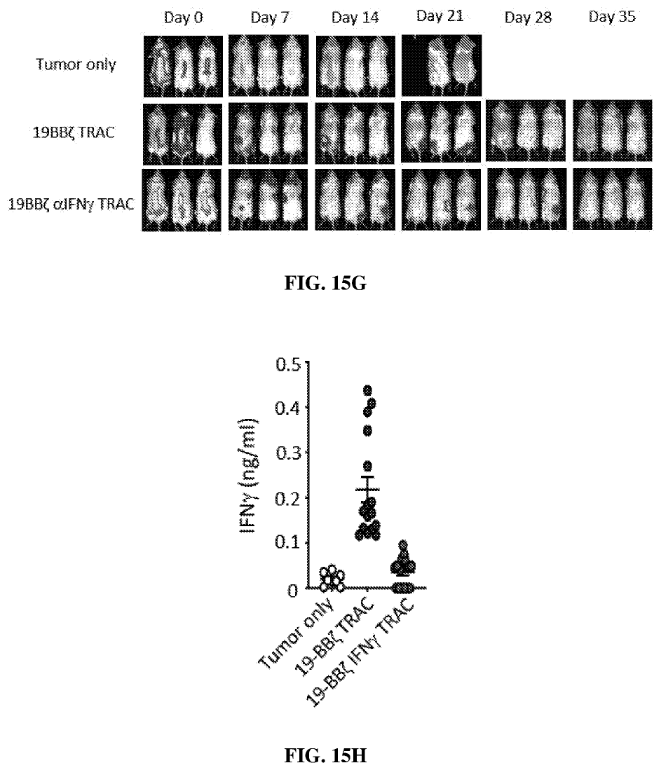

NSG mice were injected with 1e6 Jeko-1 CBG-GFP cells IV. Seven days later, mice were injected with 1e6 CAR T cells IV and tumor burden was measured by bioluminescence. Bioluminescence was measured 4, 7, 14, 21, 28 and 35 days later. On these days, mice were also bled to look for the presence of CAR T cells. Mice were injected with CD19-41BBζ CAR T cells and either not treated or treated with anti-IFNγ or control IgG antibody on the days indicated (N=3-5 mice/group; 4 donors) (FIG. 15A). Average bioluminescence flux for each group of mice over time was measured and is shown in FIG. 15B. Bioluminescent images of the mice at each time point are shown in FIG. 15C. Mice were bled 3 days post-CAR-T and serum was tested for IFNγ expression by ELISA (N=3-5 mice/group; 4 donors) (FIG. 15D). Mice were injected with BBζ TRAC or BBζ TRAC IFNγ CAR T cells 7 days post-Jeko-1 injection (FIG. 15E). Average bioluminescence flux for each group of mice over time was measured and is shown in FIG. 15F). Bioluminescent images of the mice at each time point are shown in FIG. 15G. Mice were bled 3 days post-CAR-T, serum was collected and IFNγ was assessed by ELISA (FIG. 15H).

Results in FIG. 15A-15H demonstrate that inhibition of IFNγ does not affect CAR T cell killing of lymphoma cells in mice, but does effectively reduce the levels of IFNγ in the serum as seen by ELISA.

### Blocking IFNγ Production by BBζ CAR-T Reduces Co-Inhibitory Marker Expression and Slightly Enhances Cell Proliferation In Vitro

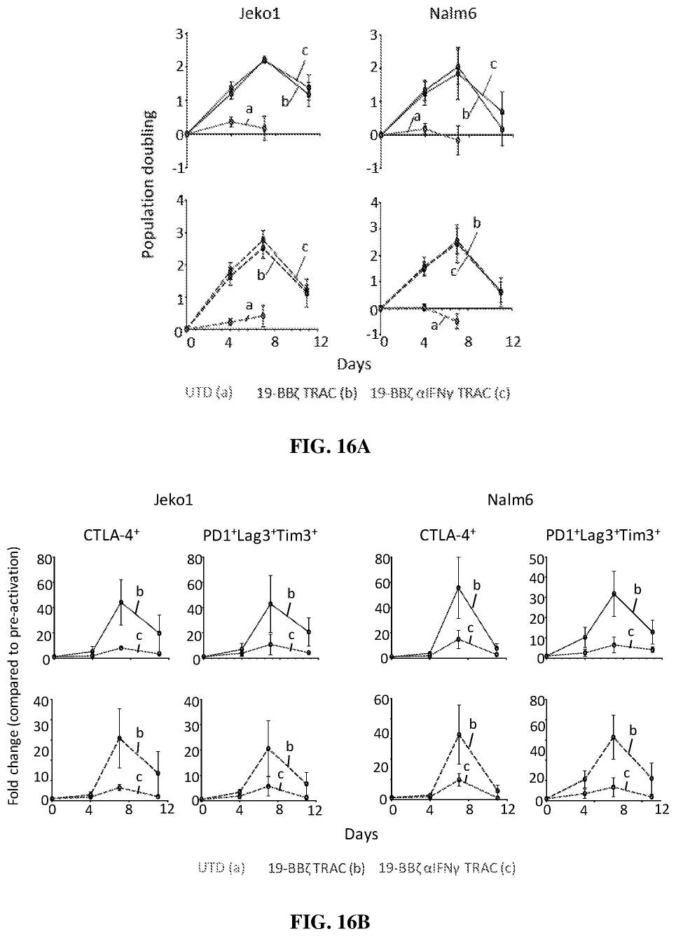

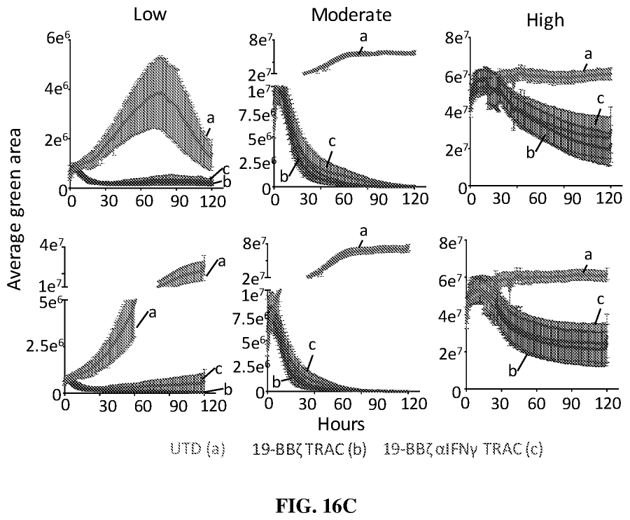

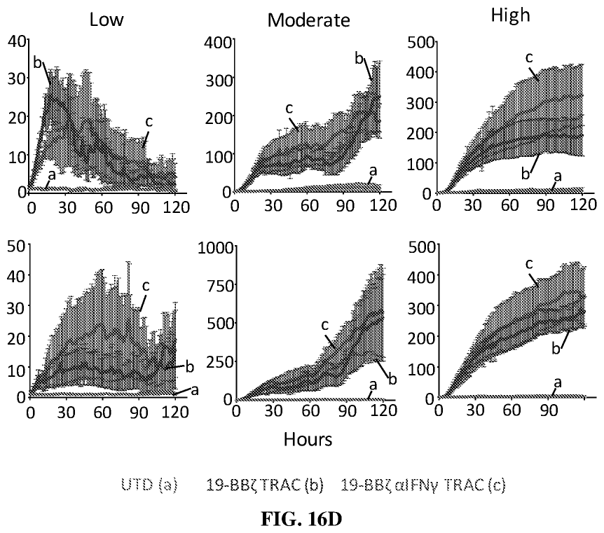

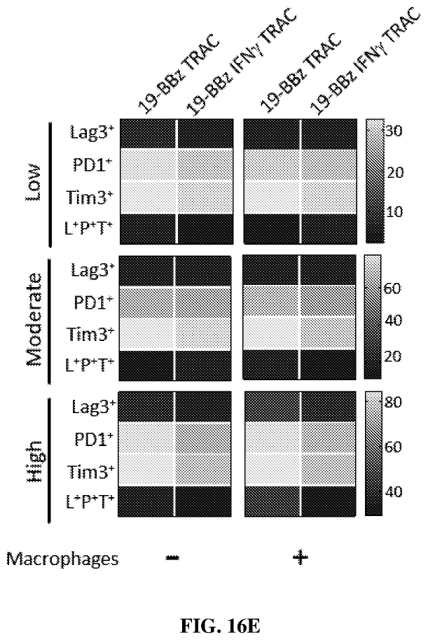

Given that the BBζ IFNγ TRAC appeared to have greater long-term persistence in Nalm6-bearing NSG mice, it was sought to determine how the loss of IFNγ affects CAR-T phenotype and expansion. KO BBζ CAR-T were generated as previously described. Ten days post-activation, cells were re-activated with irradiated Jeko-1 or Nalm6 cells at a 1:1 ratio on days 0, 4 and 7. Cells were counted prior to each re-stimulation and proliferation doubling was calculated over time (FIG. 16A). For cultures receiving macrophages (dashed lines; bottom), monocytes were culture in GMCSF for 7 days prior to combination (N=5). Prior to each re-activation, 1e5 cells were collected from cultures and analyzed by flow cytometry (N=5) (FIG. 16B). KO BBζ CAR-T were combined with Nalm6 cells at various tumor burdens (low=10E:1T, moderate=1E:1T, high=1E:10T). The bottom row was given 2,000 macrophages for a ratio of 1E:0.02M (N=5). Changes in tumor burden were tracked by Incucyte using the average green area/well (FIG. 16C). CAR-T expansion/contraction was monitored by Incucyte using the average area and fold change was calculated compared to hour 0 (N=5) (FIG. 16D). After 5 days in the Incucyte, cells were collected and stained for flow cytometry. Heatmap shows percent positive for each protein (gated on mCherry+ cells) (N=5) (FIG. 16E).

Results in FIG. 16A-16E demonstrate that while blockade of IFNγ does not affect target cell killing, it does appear to reduce the expression of co-inhibitory markers CTLA-4, PDL-1, Lag3 and Tim3 which suggests that these CAR-T will have greater proliferation/persistence. Although no changes were seen in the proliferation doubling of the cells, a trend of BBζ IFNγ KO CAR-T having greater proliferative capacity by Incucyte was observed.

### Blocking IFNγ Production by 28ζ CAR-T Reduces Co-Inhibitory Marker Expression and Greatly Enhances Cell Proliferation In Vitro

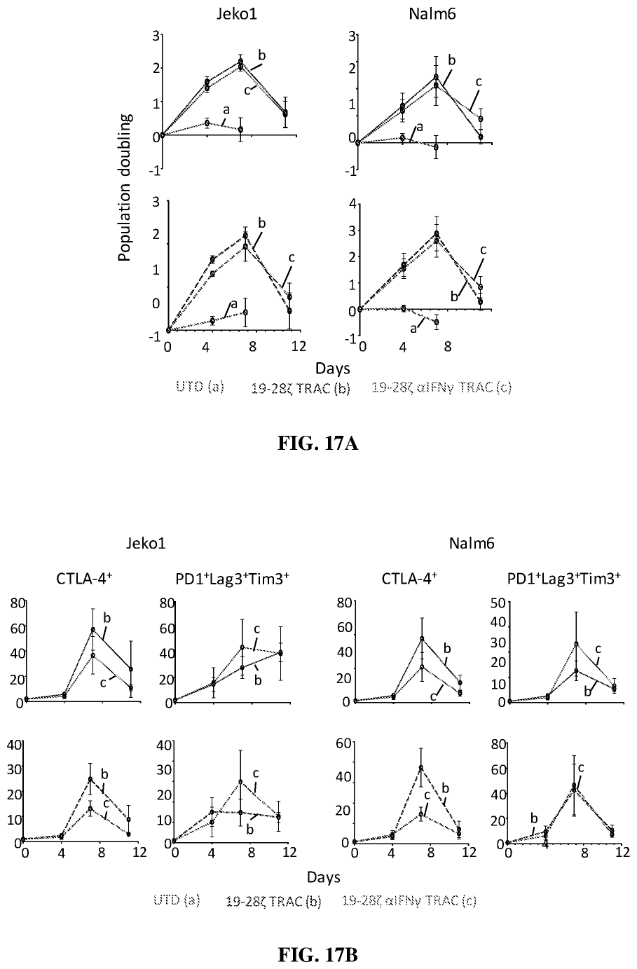

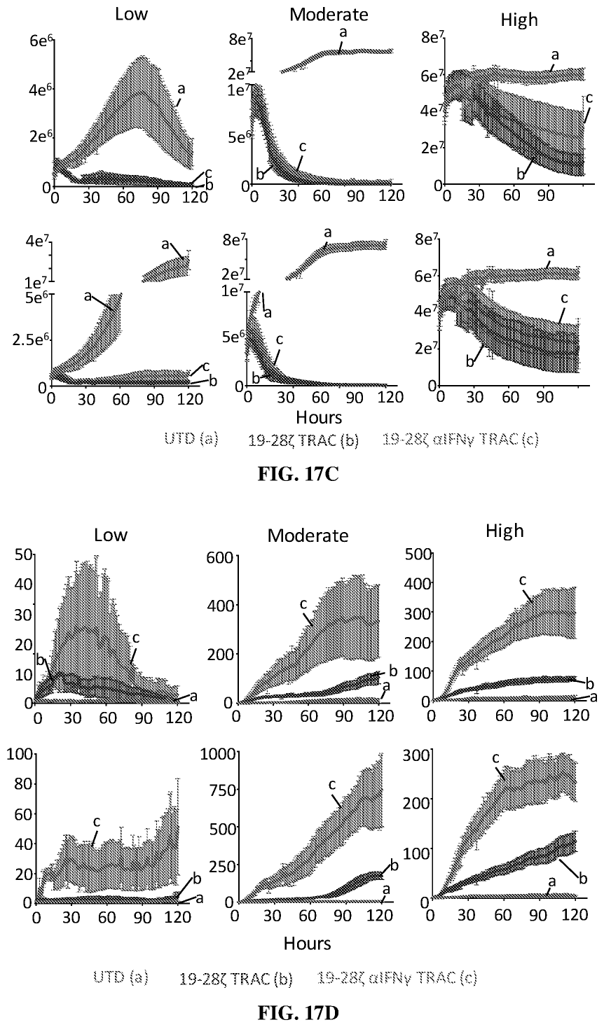

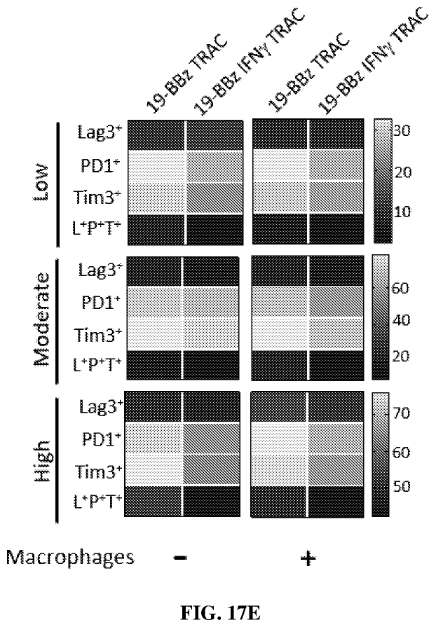

Given that the BBζ IFNγ TRAC appeared to have less exhaustion and slightly greater proliferation in response to tumor antigen, it was sought to determine how the loss of IFNγ affects 28ζ CAR-T phenotype and expansion, which is know to have more exhaustion and less persistence than BBζ. KO 28ζ CAR-T were generated as previously described. Ten days post-activation, cells were re-activated with irradiated Jeko-1 or Nalm6 cells at a 1:1 ratio on days 0, 4 and 7. Cells were counted prior to each re-stimulation and proliferation doubling was calculated over time (FIG. 17A). For cultures receiving macrophages (dashed lines; bottom), monocytes were culture in GMCSF for 7 days prior to combination (N=5). Prior to each re-activation, 1e5 cells were collected from cultures and analyzed by flow cytometry (N=5) (FIG. 17B). KO 28ζ CAR-T were combined with Nalm6 cells at various tumor burdens (low=10E:1T, moderate=1E:1T, high=1E:10T) (FIGS. 17C and 17D). The bottom row was given 2,000 macrophages for a ratio of 1E:0.02M (N=5). Changes in tumor burden were tracked by Incucyte using the average green area/well (FIG. 17C). CAR-T expansion/contraction was monitored by Incucyte using the average area and fold change was calculated compared to hour 0 (N=5) (FIG. 17D). After 5 days in the Incucyte, cells were collected and stained for flow cytometry. Heatmap shows percent positive for each protein (gated on mCherry+ cells) (N=5) (FIG. 17E).

Results in FIG. 17A-17E demonstrate that while blockade of IFNγ does not affect target cell killing, it does appear to reduce the expression of co-inhibitory markers CTLA-4, PDL-1, Lag3 and Tim3 which suggests that these CAR-T will have greater proliferation/persistence. Although no changes were seen in the proliferation doubling of the cells, a much greater expansion of 28ζ IFNγ KO CAR-T was observed in response to Nalm6 cells by Incucyte.

### Genetic Deletion of IFNγ in CAR T Cells Reduces Cytokine Chemokine Production and Adhesion Molecule Expression in the Presence of Macrophages

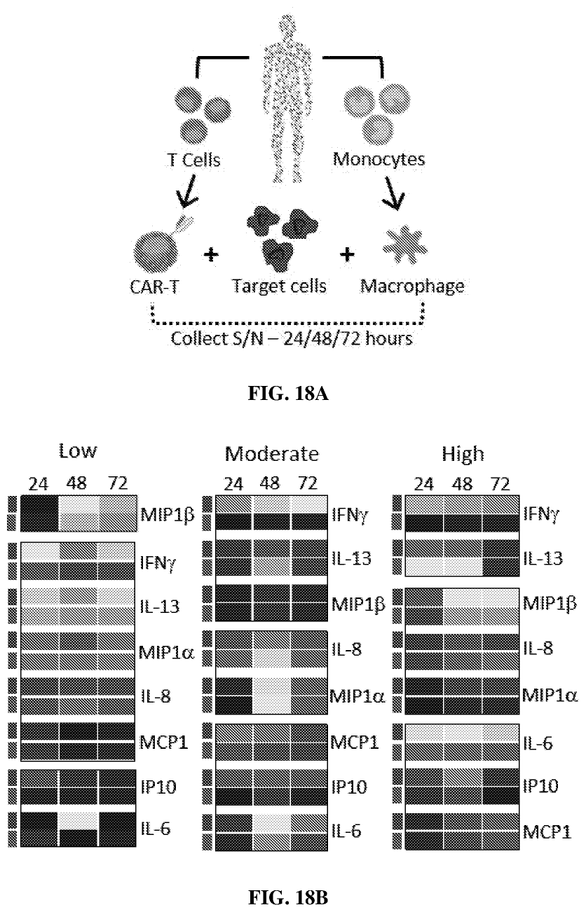

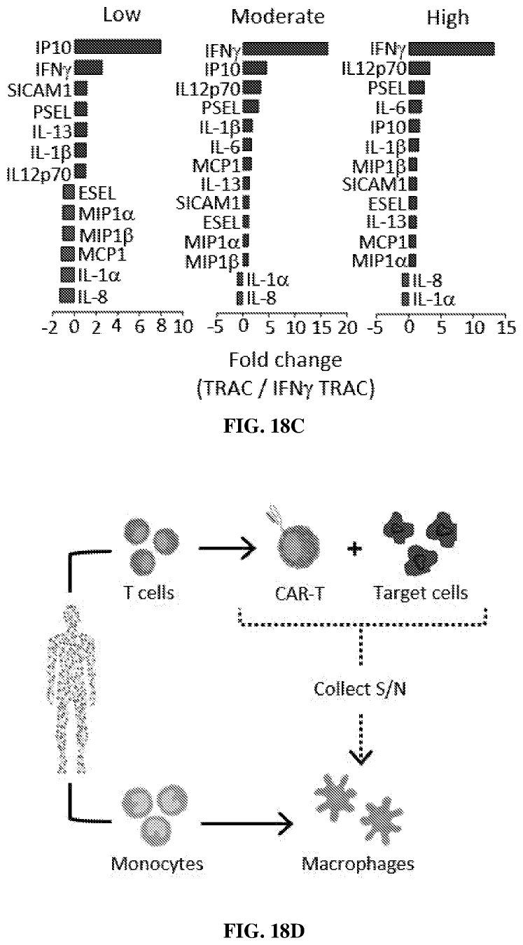

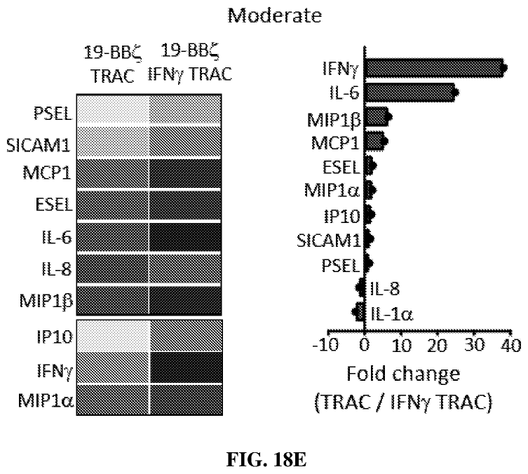

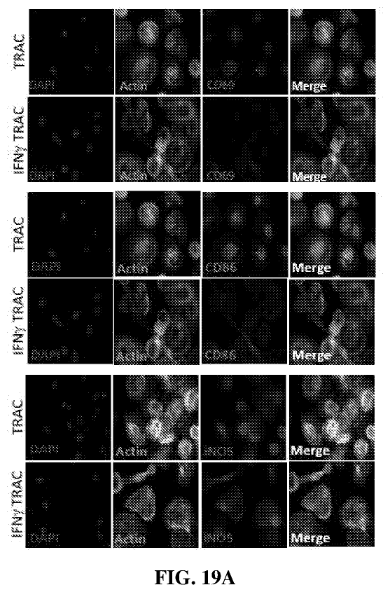

T cells and monocytes were isolated from healthy donors. T cells were activated and transduced to express the KO CAR constructs as previously discussed. Monocytes were given GMCSF for 7 days for macrophage differentiation. CAR-T and macrophages were combined with target cancer cells (Nalm6) at various tumor burden ratios: low (10E:1T:0.02M), moderate (1E:1T:0.02M) and high (1E:10T:0.02M) (FIG. 18A). Supernatant was collected 24, 48 and 72 hours post-combination. Cytokines/chemokines in the supernatant was collected and assayed by Luminex (N=4) (FIG. 18B). Fold change was calculated using TRAC divided by IFNγ TRAC (FIG. 18C). T cells and monocytes were isolated from healthy donors and expanded into CAR-T and macrophages as mentioned above. CAR-T and Nalm6 cells were combined at a 1:1 ratio for 24 hours. After 24 hours, supernatant was collected and added directly to the donor-matched macrophages. Supernatant was collected 24 hours later (see FIG. 18D). Cytokines/chemokines in supernatant was collected and assayed by Luminex (N=1) (see FIG. 18E).

Results in FIG. 18A-18E demonstrate that in the absence of IFNγ production, macrophage response to CAR-T and tumor cells is subdued as seen by reduced expression of IL-6, MCP-1, IL-1b and IP-10. This diminished macrophage response appears to yield less forward-feedback in T cells as decreased cytokines, such as GM-CSF, IL-4, IL-10, IL-12p70 and IL-13 were detected. Collectively, these data suggest that the production of IFNγ from activated CAR T cells is important for the full activation and function of macrophages and suggests that limiting its expression could reduce macrophage-mediated toxicities. Furthermore, it was discovered that the suppression of macrophage activation is not contact-dependent as direct T cell:macrophage contact was not required.

### IFNγ KO CAR-T Yield Less Activation, IFNγ Signaling and Co-Inhibitory Molecules on Macropages

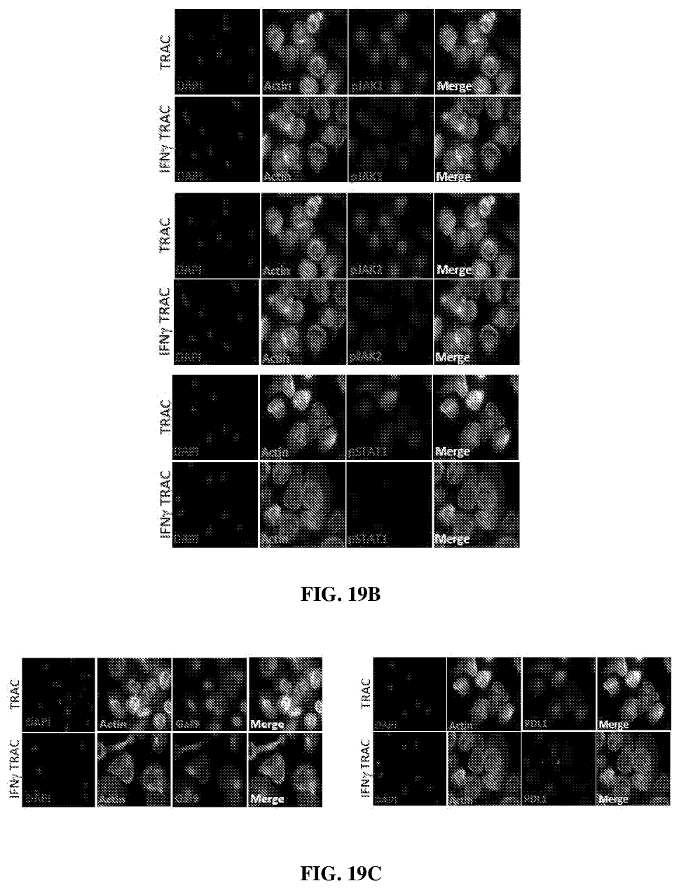

T cells and monocytes were isolated from healthy donors and expanded into BBζ KO CAR-T and macrophages as mentioned above. CAR-T and Nalm6 cells were combined at a 1:1 ratio for 24 hours. After 24 hours, supernatant was collected and added directly to the donor-matched macrophages that had been plated on glass slides for 7 days in GMCSF (30,000 cells/well). After 48 hours, macrophages were stained for macrophage activation (FIG. 19A), IFNγ signaling (FIG. 19B) and co-inhibitory markers (FIG. 19C) and imaged using a Zeiss Observer Microscope (63×; N=1).

Results in FIG. 19A-19C demonstrate that similar to the Luminex data suggesting decreased macrophage activation, the induction of activation proteins CD69 and CD86 were significantly lower in macrophages receiving IFNγ KO CAR-T supernatant compared to the TRAC CAR-T. However, iNos levels appeared similar between the groups. As expected, IFNγ signaling (pJAK1, pJAK2, pSTAT1) were all abrogated in cultures receiving IFNγ KO CAR-T supernatant. Similar to findings of reduced co-inhibitory markers on IFNγ KO CAR-T, PDL1 (but not Galectin-9) was reduced in IFNγ TRAC-treated macrophages compared to TRAC alone. Collectively, this data confirms reduced activation and IFNγ signaling in macrophages given IFNγ KO CAR-T supernatant and a subsequent decrease in PDL1.

Serum from IFNγ KO CAR-T-Treated Mice Yield Less Macrophage Activation In Vitro

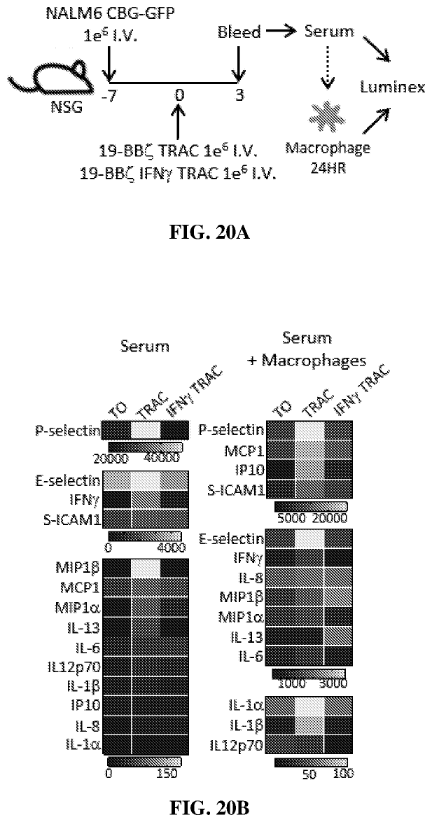

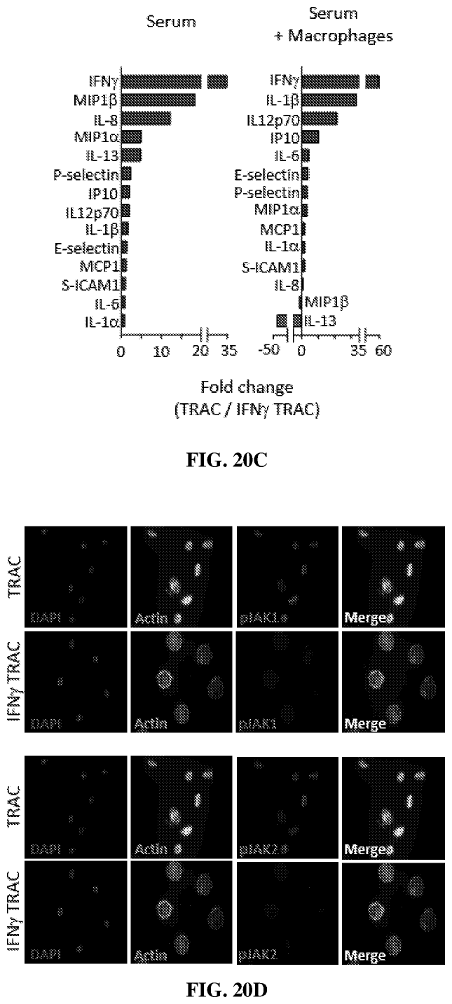

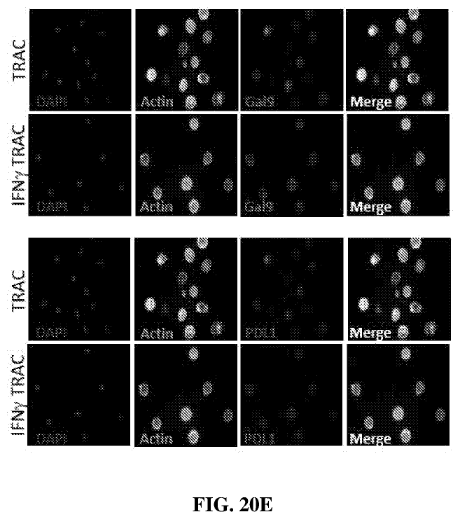

NSG mice were treated with Nalm6 and KO CAR T cells as previously described. A schematic of the experiment is depicted in FIG. 20A, showing that the serum collected from mice 3 days post-CAR-T injection was either saved directly for Luminex or added to donor-matched macrophages in vitro that were differentiated as previously discussed. Serum from mice and from macrophages were collected 24 hours later and assessed by Luminex (FIG. 20B). Fold change was calculated using TRAC/IFNγ TRAC (FIG. 20C). After collecting supernatant at 24 hours, macrophages were stained for IFNγ signaling (FIG. 20D) and co-inhibitory markers (FIG. 20E) and imaged using a Zeiss Observer Microscope (40×; N=2).

Similar to the in vitro studies, results in FIG. 19A-19C show that serum from mice treated with IFNγ KO CAR-T yielded significantly lower macrophage function in vitro compared to TRAC-treated mice. Furthermore, IFNγ signaling was impeded in these cultures as shown by reduced pJAK1 and pJAK2. Co-inhibitory markers PDL1 and Galectin-9 had a slightly lower MFI in IFNγ TRAC-treated mice.

### Materials and Methods

### CAR T Cell Production

T cells were isolated from healthy donor blood, re-suspended in R10 media (RPMI 1640+10% FBS+Pen/Strep), supplemented with 100 IU/ml IL-2 and stimulated with beads coated with anti-CD3 and anti-CD28 antibodies at a 1 T cell: 3 bead ratio. 24 hours post-activation, T cells were transduced with a lentiviral vector encoding the CAR+/−TRAC/IFNγ guides. On day 5, cells were de-beaded. In experiments where IFNγ was pharmacologically inhibited, the cells were treated with anti-IFNγ antibody for one hour on day 9 or 10 prior to performing functional assays.

### CAR T Cell Activation In Vitro

CAR T cells were activated in vitro either by treating them with PMA and Ionomycin or co-incubation with target cells. When mixed with cells, varying ratios of effector (CAR T) cells to target (cancer) cells (E:T) were used to show a dose-dependent effect. CAR T cell activation was measured by cytokine production, cell specific lysis of the target cells or expression of activation markers on the cell surface. Cytokine production was measured using an ELISA for the specific cytokine or by using a Luminex panel. IFNγ, GM-CSF, Granzyme B, and TNFα were measured using the Human DuoSet kits from R&D. Other cytokines were measured using the Th1/Th2 Luminex kits. Cell-specific lysis of target cells was measured by a change in detection of luciferase or GFP in the cell culture. Target cells express both luciferase and GFP, while CAR T cells do not, so a decrease in either when target cells are mixed with CAR T cells indicates target cell-specific lysis. Luciferase expression was measured by a luciferase assay. GFP was measured on an Incucyte, which detects the relative amount of GFP and can simultaneously measure the relative number of CAR T cells by detecting mCherry. Cell surface expression of activation markers (CD69 and CD107a) was measured by flow cytometry on the LSR Fortessa flow cytometer.

### Knocking Out IFNγ in CAR T Cells

IFNγ was knocked out in CAR T cells using a CRISPR/Cas9 system. Lentiviral constructs were designed to simultaneously express the CAR as well as small guide RNAs for IFNγ or TRAC. TRAC guides were used to target the endogenous T cell receptor. These cells were produced as described previously, but after the stimulation beads were removed, the cells were electroporated with 10 mg Cas9 mRNA. Cells with successful depletion of target genes were then identified by the absence of CD3 expression. These cells were isolated by CD3 column purification or flow-based cell sorting on day 8. CD3⁻ cells were then used for activation assays.

### CAR T Cell Killing In Vitro

CAR-T were assessed for antigen-specific cell lysis using multiple methods. For short-term killing, CAR-T were mixed with tumor cells (Jeko-1, Nalm6, Raji) at various E:T ratios for 18 hours prior to defining cell lysis by luciferase-based killing assays. To look at long-term killing, CAR-T were mixed with tumor cells at a 1:1 ratio for ACEA (60 hours) or Incucyte (5 days).

### CAR T Cell Killing In Vivo

6-8 week old NSG mice were intravenously injected with 1e6 Jeko-1 or NALM6 CBG-GFP+ cell. Seven days later, mice were left untreated (tumor only; TO) or injected with 1e6 CAR T cells IV and tumor burden was measured by bioluminescence. CAR-T were grown as previously described. Mice receiving the anti-IFNγ blocking antibody or control IgG antibody were IP injected with the appropriate solutions 1 hour prior to CAR-T injection (both given at 12 mg/kg). Antibodies were administered IP every 24 hours for the first 5 days and then maintained with 1 injection/week for the remainder of the experiment. Bioluminescence was measured 4, 7, 14, 21, 28 and 35 days later. On these days, mice were also bled to look for 1) cytokine expression by ELISA/Luminex or 2) CAR-T persistence (flow cytometry).

### Macrophage Production

Macrophages were produced from human monocytes isolated from human peripheral blood. Macrophages were generated from the same healthy donor blood as CAR T cells by isolating out the monocytes and stimulating them with GMCSF for seven days. The monocytes were then rested for 5-7 days prior to mixing them with CAR T cells and cancer cells.

### CAR-T, Macrophage and Tumor Co-Cultures

Macrophages were mixed with CAR T cells and target (cancer) cells at varying tumor burdens: low (10E:1T:0.02M), moderate (1E:1T:0.02M) or high (1E:10T:0.02M). Cells were combined and supernatant was collected at 24, 48 and 72 hours into the assay to assess cytokines. Cytokine production was measured using Human DuoSet ELISA kits or the Human Inflammatory Panel 20-plex Luminex kit. To assess contact-dependency, CAR-T and tumor cells were mixed at a moderate (1:1) ratio for 24 hours before collecting supernatant and adding it directly to GMCSF-differentiated macrophages. 24 hours later, supernatant was collected and cytokines assessed by Luminex.

Macrophage Activation from Mouse Serum

6-8 week old NSG mice were intravenously injected with 1e6 Jeko-1 or NALM6 CBG-GFP+ cell. Seven days later, mice were left untreated (tumor only; TO) or injected with 1e6 KO CAR T cells IV and tumor burden was measured by bioluminescence. CAR-T were grown as previously described. Serum was collected from mice 3 days post-CAR-T injection and either saved for Luminex or added directly to donor-matched macrophages that were differentiated with GMCSF in culture. 24 hours later, supernatant was collected and assessed for function using the the Human Inflammatory Panel 20-plex Luminex kit.

### Microscopy

Monocytes from healthy donors were plated on iBidi glass-bottom 8 well slides and kept in 5 ng/ml GMCSF for 7 days prior to use. Supernatant from CAR-T/tumor culture or serum from mice was collected and added directly to washed macrophages for 24-48 hours. Cells were fixed and permeabilized using the Molecular Probes Image iT kit according to protocol. Cells were stained with primary antibodies (non-conjugated) overnight at a concentration of 1:100-1:200. Secondary anti-rabbit antibodies conjugated to AF647 or AF549 were used at 1:500 for detection. Molecular Probes Actin Green 488 was used for actin staining and slides were mounted using Prolong Gold Antifade Reagent with DAPI. Macrophages were imaged on the Zeiss Observer Microscope at 40× or 63× with similar exposures between all samples.

All publications, patents, patent applications, publication, and database entries (e.g., sequence database entries) mentioned herein, e.g., in the Background, Summary, Detailed Description, Examples, and/or References sections, are hereby incorporated by reference in their entirety as if each individual publication, patent, patent application, publication, and database entry was specifically and individually incorporated herein by reference. In case of conflict, the present application, including any definitions herein, will control.

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the disclosure encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

Where websites are provided, URL addresses are provided as non-browser-executable codes, with periods of the respective web address in parentheses. The actual web addresses do not contain the parentheses.

In addition, it is to be understood that any particular embodiment of the present disclosure may be explicitly excluded from any one or more of the claims. Where ranges are given, any value within the range may explicitly be excluded from any one or more of the claims. Any embodiment, element, feature, application, or aspect of the compositions and/or methods of the disclosure, can be excluded from any one or more claims. For purposes of brevity, all of the embodiments in which one or more elements, features, purposes, or aspects is excluded are not set forth explicitly herein.

What is claimed is:

1. An engineered immune cell comprising a chimeric antigen receptor (CAR) or an engineered T cell receptor (TCR), wherein the engineered immune cell is deficient in interferon γ (IFNγ) expression.

2. The engineered immune cell of claim 1, wherein the engineered immune cell comprises a nucleic acid comprising a nucleotide sequence encoding the CAR or the engineered TCR operably linked to a promoter.

3. The engineered immune cell of claim 1 or claim 2, wherein the immune cell comprises a nucleotide sequence that suppresses the IFNγ gene.

4. The engineered immune cell of claim 3, wherein the nucleotide sequence that suppresses the IFNγ gene is a guide RNA (gRNA).

5. The engineered immune cell of claim 4, wherein the gRNA comprises the nucleotide sequence of CCAGAGCATCCAAAAGAGTG (SEQ ID NO: 1) or TGAAGTAAAAGGAGACAATT (SEQ ID NO: 2).

6. The engineered immune cell of claim 4 or claim 5, wherein the engineered immune cell further comprises a nucleotide sequence encoding a Cas9 nuclease or further comprises a Cas9 nuclease.

7. The engineered immune cell of claim 3, wherein the nucleotide sequence that suppresses the IFNγ gene encodes a RNAi molecule.

8. The engineered immune cell of claim 7, wherein the RNAi molecule is a siRNA, a micro-RNA, a shRNA, or an antisense oligonucleotide.

9. The engineered immune cell of claim 3, wherein the nucleotide sequence that suppresses the IFNγ gene is a ribozyme.

10. The engineered immune cell of claim 3, wherein the nucleotide sequence that suppresses the IFNγ gene encodes an enzyme selected from a transcription activator-like effector nuclease (TALEN), a zinc finger nuclease (ZFN), and a meganuclease.

11. The engineered immune cell of any one of claims 3-10, wherein the nucleotide sequence that inactivates the IFNγ gene is encoded on the same nucleic acid comprising the nucleotide sequence encoding the CAR or the engineered TCR.

12. The engineered immune cell of claim 1 or claim 2, wherein the immune cell comprises an enzyme selected from a transcription activator-like effector nuclease (TALEN), a zinc finger nuclease (ZFN), and a meganuclease.

13. The engineered immune cell of any one of claims A1-A12, wherein the CAR comprises an extracellular antigen-binding domain, a transmembrane domain, and one or more intracellular signaling domains.

14. The engineered immune cell of claim A13, wherein the extracellular antigen-binding domain comprises a single-chain antibody fragment (scFv) that binds a cell surface protein.

15. The engineered immune cell of claim A13 or claim A14, wherein the extracellular antigen-binding domain binds CD19, BCMA, TACI, CD79b, CD22, CD30, CS1, GPCR, PSMA, mesothelin, MUC1, MUC16, EGFR, IL-13Ralpha2, EGFRvIII, CD20, CD79a, or combinations thereof.

16. The engineered immune cell of any one of claims 13-15, wherein the one or more intracellular signaling domains comprise (i) an ITAM-containing signaling domains and/or (ii) one or more signaling domains from one or more co-stimulatory proteins or cytokine receptors.

17. The engineered immune cell of claim 14, wherein the ITAM-containing signaling domain is a CD3ζ signaling domain.

18. The engineered immune cell of claim 16, wherein the co-stimulatory protein or cytokine receptor is CD28, 4-1BB, 2B4, KIR, CD27, OX40, ICOS, MYD88, IL2 receptor, or SynNotch.

19. The engineered immune cell of any one of claims 16-18, wherein the one or more intracellular signaling domains comprise (i) a signaling domain of CD3ζ and/or (ii) a signaling domain from CD28 or 4-1BB.

20. The engineered immune cell of any one of claims 13-19, wherein the transmembrane domain is a CD28 transmembrane domain or CD8 transmembrane domain.

21. The engineered immune cell of any one of claims 13-20, wherein the antigen binding domain further comprises a leader sequence.

22. The engineered immune cell of any one of claims 1-21, wherein the immune cell is further deficient in endogenous TCR expression.

23. The engineered immune cell of any one of claims 1-22 wherein the immune cell is a T-cell, a NK cell, a dendritic cell, a macrophage, a B cell, a neutrophil, an eosinophil, a basophil, a mast cell, a myeloid-derived suppressor cell, a mesenchymal stem cell, a precursor thereof, or a combination thereof.

24. The engineered immune cell of claim 23, wherein the immune cell is a T cell.

25. A nucleic acid molecule comprising:
(i) a first nucleotide sequence encoding a chimeric antigen receptor (CAR) or an engineered T cell receptor (TCR); and
(ii) a second nucleotide sequence encoding an agent that suppresses interferon γ (IFNγ) gene.

26. The nucleic acid molecule of claim 25, wherein the agent that suppresses IFNγ gene is a gRNA, a siRNA, a micro-RNA, a shRNA, an antisense oligonucleotide, a ribozyme, a transcription activator-like effector nuclease (TALEN), a zinc finger nuclease (ZFN), or a meganuclease.

27. The nucleic acid of claim 26, wherein the agent that suppresses IFNγ gene is a gRNA.

28. The engineered immune cell of claim 37, wherein the gRNA comprises the nucleotide sequence of CCAGAGCATCCAAAAGAGTG (SEQ ID NO: 1) or TGAAGTAAAAGGAGACAATT (SEQ ID NO: 2).

29. The nucleic acid of any one of claims 25-28, further comprising a third nucleotide sequence encoding an agent that suppresses an endogenous TCR gene.

30. The nucleic acid molecule of claim 29, wherein the agent that suppresses the endogenous TCR gene is a gRNA, a siRNA, a micro-RNA, a shRNA, an antisense oligonucleotide, a ribozyme, a transcription activator-like effector nuclease (TALEN), a zinc finger nuclease (ZFN), or a meganuclease.

31. The nucleic acid of claim 30, wherein the agent that suppresses endogenous TCR gene is a gRNA.

32. The nucleic acid of any one of claims 25-31, wherein the CAR comprises an extracellular antigen-binding domain, a transmembrane domain, and one or more intracellular signaling domains.

33. The nucleic acid of claim 32, wherein the extracellular antigen-binding domain comprises a single-chain antibody fragment (scFv) that binds a cell surface protein.

34. The nucleic acid of claim 33 or claim 34, wherein the extracellular antigen-binding domain binds CD19, BCMA, TACI, CD79b, CD22, CD30, CS1, GPCR, PSMA, mesothelin, MUC1, MUC16, EGFR, IL-13Ralpha2, EGFRvIII, CD20, CD79a, or combinations thereof.

35. The nucleic acid of any one of claims 32-34, wherein the one or more intracellular signaling domains comprise (i) an ITAM-containing signaling domains and/or (ii) one or more signaling domains from one or more co-stimulatory proteins or cytokine receptors.

36. The engineered immune cell of claim 35, wherein the ITAM-containing signaling domain is a CD3ζ signaling domain.

37. The nucleic acid of claim 35, wherein the co-stimulatory protein or cytokine receptor is CD28, 4-1BB, 2B4, KIR, CD27, OX40, ICOS, MYD88, IL2 receptor, or SynNotch.

38. The nucleic acid of any one of claims 35-37, wherein the one or more intracellular signaling domains comprise (i) a signaling domain of CD3ζ and/or (ii) a signaling domain from CD28 or 4-1BB.

39. The nucleic acid of any one of claims 32-38, wherein the transmembrane domain is a CD28 transmembrane domain.

40. The nucleic acid of any one of claims 32-39, wherein the antigen binding domain further comprises a leader sequence.

41. The nucleic acid of any one of claims 25-40, wherein the nucleic acid is a vector.

42. The nucleic acid of claim 41, wherein the vector is an AAV, a lentiviral vector, or a retroviral vector.

43. A method comprising delivering the nucleic acid of any one of claims 25-33 to an immune cell.

44. The method of claim 43, wherein the immune cell is a T-cell, a NK cell, a dendritic cell, a macrophage, a B cell, a neutrophil, an eosinophil, a basophil, a mast cell, a myeloid-derived suppressor cell, a mesenchymal stem cell, a precursor thereof, and a combination thereof.

45. The method of claim 44, wherein the immune cell is a T cell.

46. The method of claim 44 or claim 45, wherein the second nucleotide sequence encodes a gRNA and the method further comprises delivering to the immune cell a nucleotide sequence encoding a Cas9 nuclease or delivering to the immune cell a Cas9 nuclease.

47. A method of treating cancer or an autoimmune disease, the method comprising administering to a subject in need thereof an effective amount of the engineered immune cell of any one of claims 1-24.

48. A method of reducing cytokine release associated with CAR-T cell therapy, the method comprising administering to a subject in need thereof an effective amount of the engineered immune cell of any one of claims 1-24.

49. A method comprising administering to a subject the engineered immune cell of any one of claims 1-24.

50. The method of any one of claims 47-49, wherein the subject is a human subject.

51. The method of any one of claims 47-50, wherein the administering is via infusion.

52. The method of any one of claims 47-51, wherein the engineered immune cell is allogeneic or autologous.

53. The method of any one of claims 47-52, wherein the level of inflammatory cytokines, chemokines, and/or adhesion molecules produced in the subject are reduced, compared to that of a subject administered an engineered immune cell not deficient in IFNγ expression.

54. The method of claim 53, wherein the inflammatory cytokines, the chemokines, or the adhesion molecules are selected from: IL-4, IL-10, IL-12, IL-13, MIP1α, MIP1β, MCP1, IP10, E-selectin, P-selection, PSEL, IL-1beta, IL12p70 and SICAM1.

55. The method of claim 53 or claim 54, wherein the reduction of the level of the inflammatory cytokines, chemokines, and/or adhesion molecules is in cancer microenvironment, circulation or central nervous system.

56. The method of any one of claims 47-55, wherein the cancer is lymphoma.

57. The method of claim 56, wherein the cancer is mantle cell lymphoma.

58. The method of anyone of claims 47-57, wherein the cancer is leukemia.

59. The method of claim 58, wherein the cancer is acute lymphoblastic leukemia.
